(19) United States
(12) Patent Application Publication
(10) Pub. No.: US 2022/0323448 A1
(43) Pub. Date: Oct. 13, 2022

(54) COMPOSITIONS AND METHODS FOR TREATING EYES AND METHODS OF PREPARATION

(71) Applicant: Ocular Science, Inc., Manhattan Beach, CA (US)

(72) Inventors: Anthony SAMPIETRO, Hermosa Beach, CA (US); Damien GOLDBERG, Manhattan Beach, CA (US); Amy FROST, Stevensville, MT (US); Brian HOLDORF, Missoula, MT (US); Francis MAH, Del Mar, CA (US)

(21) Appl. No.: 17/722,684

(22) Filed: Apr. 18, 2022

Related U.S. Application Data
(63) Continuation-in-part of application No. 16/751,177, filed on Jan. 23, 2020, now Pat. No. 11,395,825.
Application No. 16/751,177 is a continuation of application No. 15/971,936, filed on May 4, 2018, now abandoned.
Application No. 15/971,936 is a continuation-in-part of application No. 17/222,419, filed on Apr. 5, 2021.
(60) Provisional application No. 62/646,287, filed on Mar. 21, 2018.
(60) Provisional application No. 62/621,299, filed on Jan. 24, 2018.
(60) Provisional application No. 62/501,390, filed on May 4, 2017.
(60) Provisional application No. 63/037,171, filed on Jun. 10, 2020.

(51) Int. Cl. A61K 31/5377; A61K 31/216; A61K 31/4709; A61K 31/573; A61K 45/06; A61K 9/00; A61P 27/02

(52) U.S. Cl. CPC A61K 31/5377; A61K 31/216; A61K 31/4709; A61K 31/573; A61K 45/06; A61K 9/0048; A61P 27/02

(57) ABSTRACT

Provided herein are certain preservative-free ophthalmic formulations for post-operative ocular care. Pharmaceutical compositions, methods for treating various issues of the eyes, and methods of preparing such compositions are described. These pharmaceutical compositions may be for treating glaucoma, in preparation of eye surgery, during eye surgery, various post-op care (e.g., after cataract surgery, laser eye surgery, and the like), for treating dry eyes, and/or for promoting eyelash growth. These pharmaceutical compositions may comprise such active ingredients (APIs) as: timolol, latanoprost, brimonidine tartrate, dorzolamide, moxifloxacin HCl, dexamethasone PO₄, phenylephrine HCl, lidocaine HCl, ketorolac tromethamine, bromfenac, prednisolone PO₄, gatifloxacin, amniotic cytokine extract (ACE), prostaglandin E2 (PGE2), and combinations thereof.

### TECHNICAL FIELD OF THE INVENTION

The present invention relates to compositions for treating eyes, methods for treating eyes, and methods for preparing said compositions and more specifically to treating the eyes for glaucoma, for post-op care after various eye surgeries, and/or for dry eyes.

### BACKGROUND OF THE INVENTION

Today (circa 2018) many people face various problems with their eyes, such as glaucoma, preparation for eye surgery, care during eye surgery, necessary post-op care after various eye surgeries (e.g., after cataract surgery, Lasik surgery, Lasik like surgery, laser surgery, and the like), dry eyes, and the like. Typical treatments for such eye problems include treatments, via eye drops and/or intra-cameral injections, of a variety of different packaged medications, typically each with one given API (active pharmaceutical ingredient), which may result in patient compliance problems because the patient needs to use multiple eye droppers for a given dosing (or multiple injections for a given dosing), and increased patient cost problems.

It would be desirable if such different packaged medications could be formulated to exist in a single package or delivery device, which may be improve patient compliance and reduce costs to the patient. Dosing from a single container (that combines APIs) as opposed to multiple containers where the APIs are in separate and different containers, also exposes the patient to fewer preservatives. It would also be desirable if such a combined single packaging or delivery device, also increased efficacy and/or minimized side-effects.

Post-operative care for ocular surgeries involves the administration of several medications. These medications are typically administered in the form of individual bottles of eye drops containing one active pharmaceutical ingredient (API) each, resulting in poor patient compliance due to the high number of dropper bottles, increased patient cost, and increased ocular exposure to preservatives. Consequently, there is a need for the development of preservative-free ocular formulations that contain multiple APIs in a single dropper bottle.

### SUMMARY OF THE INVENTION

To minimize the limitations in the prior art, and to minimize other limitations that will be apparent upon reading and understanding the present specification, embodiments of the present invention may describe pharmaceutical compositions, methods for treating various issues of the eyes, and methods of preparing such compositions are described. These pharmaceutical compositions may be for treating glaucoma, various post-op care (e.g., after cataract surgery, laser eye surgery, and the like), and/or for treating dry eyes. These pharmaceutical compositions may comprise such active ingredients (APIs) as: timolol, latanoprost, brimonidine tartrate, dorzolamide, moxifloxacin HCl, dexamethasone PO₄, ketorolac tromethamine, phenylephrine HCl, lidocaine HCl, bromfenac, prednisolone PO₄, gatifloxacin, amniotic cytokine extract (ACE), prostaglandin E2 (PGE2), and combinations thereof.

It is an objective of the present invention to provide pharmaceutical compositions and/or methods of treating eyes with given pharmaceutical compositions.

It is another objective of the present invention to provide pharmaceutical compositions and/or methods of treating eyes with given pharmaceutical compositions that result in increased patient compliance, e.g., by minimizing the number of treatments or by minimizing the number of separate medications being used.

It is another objective of the present invention to provide pharmaceutical compositions and/or methods of treating eyes with given pharmaceutical compositions that result in improved efficacy.

It is another objective of the present invention to provide pharmaceutical compositions and/or methods of treating eyes with given pharmaceutical compositions that result in minimal side-effects.

It is yet another objective of the present invention to provide pharmaceutical compositions and/or methods of treating eyes with given pharmaceutical compositions that result in minimal or better cost savings for the patient, e.g., by minimizing the number of separate medications which must be administered to the eyes.

These and other advantages and features of the present invention are described herein with specificity so as to make the present invention understandable to one of ordinary skill in the art, both with respect to how to practice the present invention and how to make the pre-sent invention.

Provided herein, in one aspect, is a method for treating an ocular condition of an eye, comprising administering a pharmaceutical composition at, in, or around the eye via a delivery device and per a predetermined dosing regimen, wherein:

the pharmaceutical composition is free of preservatives;

the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%;
   wherein these percentages are with respect to weight per volume; and
the ocular condition is care after cataract surgery, care after LASIK surgery, care for a retina of the eye after cataract surgery, care for a retina of the eye after retina surgery, in preparation for an intraocular procedure, or during the intraocular procedure.

In some embodiments, the delivery device is an eye dropper. In some embodiments, the eye dropper is a multidose eye dropper. In some embodiments, the multidose eye dropper is (i) a dropper bottle for dispensing predetermined metered quantities of liquid, the dropper bottle comprising a non-return position preventing the liquid from flowing back into the dropper bottle; or (ii) an Ophthalmic Squeeze Dispenser (OSD) comprising a sealing closure member that closes a dispenser orifice when the liquid present near the dispenser orifice is at a pressure less than a predetermined threshold.

In some embodiments, the predetermined dosing regimen is once per day, twice per day, three times per day, once every other day, once per week, once every other week, or once monthly.

In some embodiments, the pharmaceutical composition comprises prednisolone PO₄ about 1%, moxifloxacin HCl about 0.5%, and bromfenac about 0.075%.

In some embodiments, the pharmaceutical composition comprises prednisolone PO₄ about 1% and moxifloxacin HCl about 0.5%.

In some embodiments, the pharmaceutical composition comprises moxifloxacin HCl about 0.5% and bromfenac about 0.075%.

In some embodiments, the pharmaceutical composition comprises difluprednate about 0.05%, moxifloxacin HCl about 0.5%, and bromfenac about 0.075%.

Provided herein, in another aspect, is a method for treating an ocular condition of an eye, comprising administering a pharmaceutical composition at, in, or around the eye via a delivery device and per a predetermined dosing regimen; wherein the pharmaceutical composition comprises at least two active pharmaceutical ingredients compounded and stored in communication with each other; wherein the pharmaceutical composition is free of preservatives; wherein the method is more effective as compared against a preexisting method; wherein the preexisting method administers the at least two active pharmaceutical ingredients from at least two separate and different containers; and wherein the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%;

wherein these percentages are with respect to weight per volume.

In some embodiments, the ocular condition is one or more of: care after cataract surgery, care after LASIK surgery, care for a retina of the eye after cataract surgery, care for a retina of the eye after retina surgery, in preparation for an intraocular procure, or during an intraocular procedure.

In some embodiments, the delivery device is an eye dropper. In some embodiments, the eye dropper is a multidose eye dropper. In some embodiments, the multidose eye dropper is (i) a dropper bottle for dispensing predetermined metered quantities of liquid, the dropper bottle comprising a non-return position preventing the liquid from flowing back into the dropper bottle; or (ii) an Ophthalmic Squeeze Dispenser (OSD) comprising a sealing closure member that closes a dispenser orifice when the liquid present near the dispenser orifice is at a pressure less than a predetermined threshold.

In some embodiments, the predetermined dosing regimen is once per day, twice per day, three times per day, once every other day, once per week, once every other week, or once monthly.

In some embodiments, the pharmaceutical composition comprises prednisolone PO₄ about 1%, moxifloxacin HCl about 0.5%, and bromfenac about 0.075%.

In some embodiments, the pharmaceutical composition comprises prednisolone PO₄ about 1% and moxifloxacin HCl about 0.5%.

In some embodiments, the pharmaceutical composition comprises moxifloxacin HCl about 0.5% and bromfenac about 0.075%.

In some embodiments, the pharmaceutical composition comprises difluprednate about 0.05%, moxifloxacin HCl about 0.5%, and bromfenac about 0.075%.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 may depict a flow diagram of a method; wherein this method may comprise steps for compounding and/or filling a given pharmaceutical composition.

FIG. 2 may depict a flow diagram of another method; wherein this method may comprise steps for compounding and/or filling a particular pharmaceutical composition.

FIG. 3 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 3 may depict some baseline characteristics.

FIG. 4 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 4 may depict a 2-sided 95% confidence interval (CI).

FIG. 5 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 5 may depict mean change from baseline in IOP (intraocular pressure) over time.

FIG. 6 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 6 may depict morning IOP change from baseline.

FIG. 7 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 7 may depict total corneal staining scale (TCS) information.

FIG. 8 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 8 may depict mean change from baseline in TCS over time.

FIG. 9 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 9 may depict visual acuity change from baseline.

FIG. 10 may depict a table from a study on a pharmaceutical composition comprising prednisolone PO₄ (phosphate) 1% and gatifloxacin 0.5%; wherein FIG. 10 may depict UCDVA (uncorrected distance visual acuity) frequencies.

FIG. 11 may depict a table from a study on a pharmaceutical composition comprising prednisolone PO₄ (phosphate) 1% and gatifloxacin 0.5%; wherein FIG. 11 may depict UCDVA (uncorrected distance visual acuity) frequencies.

FIG. 12 may depict a table from a study on a pharmaceutical composition comprising prednisolone PO₄ (phosphate) 1% and gatifloxacin 0.5%; wherein FIG. 12 may depict UCVA (uncorrected visual acuity) outcomes summary.

FIG. 13 may depict a table from a study on a pharmaceutical composition comprising prednisolone PO₄ (phosphate) 1% and gatifloxacin 0.5%; wherein FIG. 13 may depict AC Cell results.

FIG. 14 may depict a table from a study on a pharmaceutical composition comprising prednisolone PO₄ (phosphate) 1% and gatifloxacin 0.5%; wherein FIG. 14 may depict AC flare results.

FIG. 15 may depict a table from a study on a pharmaceutical composition comprising prednisolone PO₄ (phosphate) 1% and gatifloxacin 0.5%; wherein FIG. 15 may depict total corneal staining (TCS) results.

FIG. 16 may depict a table from a study on a pharmaceutical composition comprising prednisolone PO₄ (phosphate) 1% and gatifloxacin 0.5%; wherein FIG. 16 may depict IOP (intraocular pressure) change from baseline.

FIG. 17 may depict a table from a study on a pharmaceutical composition comprising PGE2 and active cytokines from amniotic cytokine extract; wherein FIG. 17 may depict summary of week 4 study results.

FIG. 18 may depict a table from a study on a pharmaceutical composition comprising PGE2 and active cytokines from amniotic cytokine extract; wherein FIG. 18 may depict summary of week 12 study results.

FIG. 19 may depict a table from a study on a pharmaceutical composition comprising PGE2 and active cytokines from amniotic cytokine extract; wherein FIG. 19 may depict mean change from baseline at 4 weeks, 2-sided 95% confident interval (CI).

FIG. 20 may depict a table from a study on a pharmaceutical composition comprising PGE2 and active cytokines from amniotic cytokine extract; wherein FIG. 20 may depict mean change from baseline at 8 weeks, 2-sided 95% confident interval (CI).

FIG. 21 may depict a table from a study on a pharmaceutical composition comprising PGE2 and active cytokines from amniotic cytokine extract; wherein FIG. 21 may depict mean change from baseline at 12 weeks, 2-sided 95% confident interval (CI).

FIG. 22 may depict a table from a study on a pharmaceutical composition comprising PGE2 and active cytokines from amniotic cytokine extract; wherein FIG. 22 may depict mean change (95% CI) in VAS over time.

FIG. 23 may depict a table from a study on a pharmaceutical composition comprising PGE2 and active cytokines from amniotic cytokine extract; wherein FIG. 23 may depict mean change (95% CI) in corneal staining over time.

### DETAILED DESCRIPTION OF THE INVENTION

Below is a table listing at least fifteen separate and distinct pharmaceutical compositions by their respective APIs (active pharmaceutical ingredients), as well as listing at least one treatment purpose and at least one delivery device or method that may be contemplated embodiments of the present invention:

| | | Sample Treatment | Sample Delivery |
|---|---|---|---|
| | Compositions of APIs | Purpose | Device/Method |
| 1 | Timolol 0.5%, Latanoprost 0.005% | for treating | eye drops |
| | | glaucoma | |
| 2 | Timolol 0.5%, Brimonidine Tartrate 0.2%, | for treating | eye drops |
| | Dorzolamide 2% | glaucoma | |
| 3 | Timolol 0.5%, Brimonidine Tartrate 0.2%, | for treating | eye drops |
| | Dorzolamide 2%, Latanoprost 0.005% | glaucoma | |
| 4 | Moxifloxacin HCl 0.5% | post-op care after | intra-cameral |
| | | cataract surgery | injection |
| 5 | Dexamethasone PO₄ 0.1%, Moxifloxacin HCl | post-op care after | intra-cameral |
| | 0.5% | cataract surgery | injection |
| 6 | Dexamethasone PO₄ 0.1%, Moxifloxacin HCl | post-op care after | intra-cameral |
| | 0.5%, Ketorolac Tromethamine 0.5% | cataract surgery | injection |
| 7 | Dexamethasone PO₄ 0.1%, Moxifloxacin HCl | post-op care after | intra-cameral |
| | 0.5%, Bromfenac 0.07% | cataract surgery | injection |
| 8 | Prednisolone PO₄ 1%, Gatifloxacin 0.5% | post-op care after | eye drops |
| | | Lasik surgery | |
| 9 | Prednisolone PO₄ 1%, Ketorolac Tromethamine | post-op care for | eye drops |
| | 0.5% | retina after surgery | |
| 10 | Prednisolone PO₄ 1%, Ketorolac Tromethamine | post-op care after | eye drops |
| | 0.5%, Gatifloxacin 0.5% | cataract surgery | |
| 11 | Prednisolone PO₄ 1%, Bromfenac 0.07% | post-op care for | eye drops |
| | | retina after surgery | |
| 12 | Prednisolone PO₄ 1%, Gatifloxacin 0.5%, | post-op care after | eye drops |
| | Bromfenac 0.07% | cataract surgery | |
| 13 | Prostaglandin E2 (PGE2) | for treating dry eyes | eye drops |
| 14 | Phenylephrine HCl 1.5%, Lidocaine HCl 1%, | pre-op or during | intra-cameral |
| | Ketorolac Tromethamine 0.3% | cataract surgery | injection |
| 15 | Phenylephrine HCl 1.5%, Lidocaine HCl 1%, | pre-op or during | intra-cameral |
| | Bromfenac 0.01% | cataract surgery | injection |

HCl in the above formulations may be a standard chemical abbreviation for hydrochloride or hydrochloric acid. PO₄ in the above formulations may be a standard chemical abbreviation for phosphate.

Each of the above of the at least fifteen pharmaceutical compositions are discussed further and in more detail below.

Discussion of Timolol 0.5%, Latanoprost 0.005%

In some embodiments, a pharmaceutical composition may comprise at least two active ingredients (APIs), timolol maleate (timolol) and latanoprost.

In some embodiments, timolol maleate may also be known as timolol, timolol hemihydrate, and timolol ophthalmic solution. In some embodiments, timolol maleate may be a non-selective beta-adrenergic receptor blocking agent. Mechanism of action may be through blockage of both beta1 and beta2-adrenergic receptors and reduction of intraocular pressure by reducing aqueous humor production or increasing outflow of aqueous humor. In some embodiments, timolol maleate may work to decrease fluid production and pressure inside the eye. In some embodiments, timolol maleate may be used for treating increased pressure in the eye (ocular hypertension) and/or glaucoma (e.g., open-angle glaucoma). In some embodiments, timolol maleate may be indicated for the treatment of elevated intraocular pressure (IOP) in patients with ocular hypertension and/or open-angle glaucoma.

In some embodiments, the timolol may be present in the pharmaceutical composition at 0.5%; that is each mL (milliliter) of the pharmaceutical composition may contain 5 mg (milligram) of timolol (6.8 mg of timolol maleate).

In some embodiments, latanoprost may be known as latanoprost ophthalmic solution. In some embodiments, latanoprost may be in the prostaglandin analogue family of medication; i.e., a prostaglandin analog. Mechanism of action may be through a prostaglandin F2-alpha analog to reduce intraocular pressure by increasing the outflow of the aqueous humor. In some embodiments, the latanoprost may work by increasing the outflow of aqueous fluid from the eyes through the uveoscleral tract. In some embodiments, latanoprost may be indicated for the treatment of elevated intraocular pressure (IOP) in patients with open-angle glaucoma and/or ocular hypertension. In some embodiments, the latanoprost may be used for treating increased pressure in the eye (ocular hypertension) and glaucoma (e.g., open-angle glaucoma). In some embodiments, the latanoprost may be present in the pharmaceutical composition at 0.005%; that is each mL of pharmaceutical composition may contain 50 mcg (microgram) of latanoprost.

In some embodiments, this pharmaceutical composition may also comprise sodium chloride (NaCl) for purposes of yielding an isotonic, buffered, aqueous solution.

In some embodiments, this pharmaceutical composition may also comprise sodium chloride and/or sodium hydroxide (NaOH) for purposes of adjusting pH of the resulting pharmaceutical composition to a target pH of 6.7. In some embodiments, this pharmaceutical composition may also comprise hydrochloric acid (HCl) for pH adjustment purposes.

In some embodiments, this pharmaceutical composition may comprise benzalkonium chloride (BAK), in addition to the timolol maleate and the latanoprost. In some embodiments, BAK may be a detergent, a quaternary ammonium compound with a broad range of antimicrobial activity. In some embodiments, BAK may be a preservative in the pharmaceutical composition.

In some embodiments, a carrier and/or a solvent of the timolol 0.5% and the latanoprost 0.005% pharmaceutical composition may be water. In some embodiments, this water may be sterile water, water for irrigation, water for injection (WFI), or the like.

In some embodiments, the timolol 0.5% and the latanoprost 0.005% pharmaceutical composition may be delivered to the eyes of a patient via use eye drops from an eye dropper.

In some embodiments, the timolol 0.5% and the latanoprost 0.005% pharmaceutical composition may be used for treating glaucoma in the eye(s).

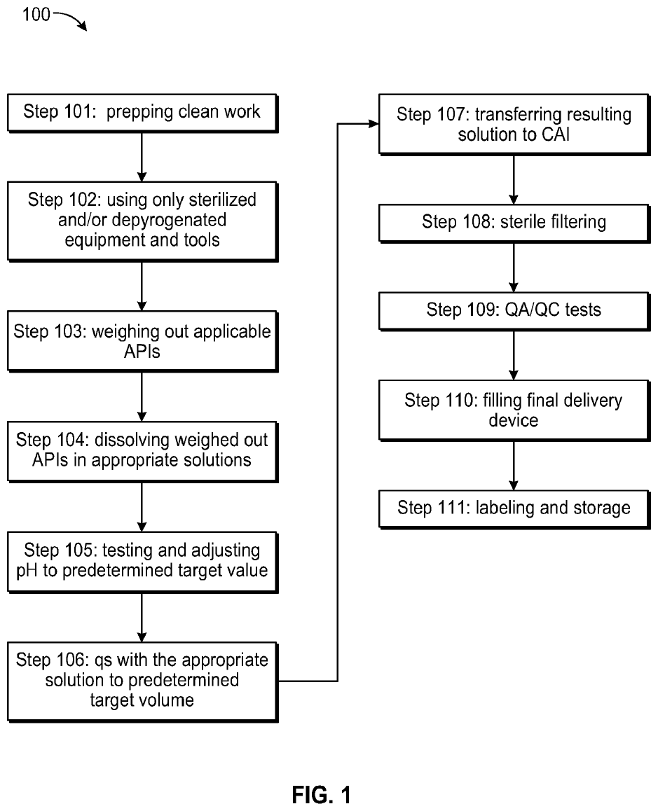

In some embodiments, compounding the pharmaceutical composition comprising the timolol 0.5% and the latanoprost 0.005% may comprise steps of: (step 101) prepping clean work area (e.g., cleaning and/or disinfecting); (step 102) using only sterilized and/or depyrogenated equipment; (step 103) weighing applicable APIs (e.g., timolol maleate and latanoprost) in a powder hood (with the 0.5% and the 0.005% targets in mind); (step 104) dissolving weighed out API powders in sterile water (or WFI) (with the 0.5% and the 0.005% targets in mind); (step 105) testing and adjusting the pH to a target of 6.7 via use of sodium chloride, sodium hydroxide, and pH meter (calibrated); (step 106) qs (“quantity sufficient”) with the sterile water (or WFI) with the 0.5% and 0.005% targets in mind; (step 107) transferring resulting solution to a compounding aseptic isolator (CAI); (step 108) sterile filtering (e.g., a 0.22 micron filter) the resulting solution to yield the pharmaceutical composition comprising the timolol 0.5% and the latanoprost 0.005%; (step 109) QA/QC (quality assurance/quality control) tests, such as bubble point testing, sterility testing, and/or endotoxin testing; (step 110) and filling final delivery device, e.g., a sterile ophthalmic dropper bottle (e.g., a “drop-tainer,” “steri-dropper,” or the like); and (step 111) of label and storage. See e.g., FIG. 1. In some embodiments, the final delivery device, e.g., the sterile ophthalmic dropper bottle, may be light resistant.

For example, and without limiting the scope of the present invention, a 200 mL batch of the pharmaceutical composition comprising the timolol 0.5% and the latanoprost 0.005% may comprise: 1.360 grams of timolol maleate; 1.030 grams of latanoprost stock solution (at 10 mg/g); 1.600 grams of sodium chloride; 2.000 mL of BAK 1%; and the balance of sterile water (or WFI).

In some embodiments, the pharmaceutical composition may comprise timolol 0.5%, latanoprost 0.005%, sodium chloride, BAK, sodium hydroxide, and water; wherein that pharmaceutical composition may have a pH of 6.7.

Discussion of Timolol 0.5%, Brimonidine Tartrate 0.2%, Dorzolamide 2%

In some embodiments, a pharmaceutical composition may comprise at least three active ingredients (APIs), timolol maleate (timolol), brimonidine tartrate, and dorzolamide.

In some embodiments, the timolol may be as discussed above.

In some embodiments, brimonidine tartrate may also be known as brimonidine and brimonidine ophthalmic solution. Mechanism of action may be through relative selective alpha-2 adrenergic agonist causing reduction of aqueous humor formation and increased uveoscleral outflow. In some embodiments, the brimonidine tartrate may be a relatively selective alpha-2 adrenergic agonist. In some embodiments, the brimonidine tartrate may function via a dual mechanism of action by reducing aqueous humor production and increasing uveoscleral outflow. In some embodiments, brimonidine tartrate may be indicated for the treatment of elevated intraocular pressure (IOP) in patients with open-angle glaucoma and/or ocular hypertension. In some embodiments, the brimonidine tartrate may be used for treating increased pressure in the eye (ocular hypertension) and glaucoma (e.g., open-angle glaucoma). In some embodiments, the brimonidine tartrate may be present in the pharmaceutical composition at 0.2%; that is each mL of the pharmaceutical composition may contain 2 mg of brimonidine tartrate.

In some embodiments, the dorzolamide may be known as dorzolamide ophthalmic solution. Mechanism of action may be through reversible inhibition of the enzyme carbonic anhydrase resulting in reduction of hydrogen ion secretion at the renal tubule and an increased renal excretion of sodium, potassium, bicarbonate, and water to decrease production of aqueous humor. Dorzolamide may also inhibit carbonic anhydrase in the central nervous system (CNS) to retard abnormal and excessive discharge from CNS neurons. In some embodiments, the dorzolamide may be a carbonic anhydrase inhibitor. In some embodiments, the dorzolamide may work by decreasing fluid production and pressure inside the eye. In some embodiments, the dorzolamide may be indicated for the treatment of elevated intraocular pressure (IOP) in patients with ocular hypertension and/or open-angle glaucoma. In some embodiments, the dorzolamide may be used for treating increased pressure in the eye (ocular hypertension) and/or glaucoma (e.g., open-angle glaucoma). In some embodiments, the dorzolamide may be present in the pharmaceutical composition as dorzolamide HCl. In some embodiments, the dorzolamide may be present in the pharmaceutical composition at 2%; that is, each mL of the pharmaceutical composition may contain 20 mg of dorzolamide (e.g., 22.26 mg of dorzolamide HCl).

In some embodiments, this pharmaceutical composition may also comprise sodium phosphate monobasic.

In some embodiments, this pharmaceutical composition may also comprise sodium phosphate monobasic and/or sodium hydroxide for purposes of adjusting pH of the resulting pharmaceutical composition to a target pH of 5.8.

In some embodiments, this pharmaceutical composition may comprise BAK, in addition to the timolol maleate, the brimonadine tartrate, and the dorzolamide. In some embodiments, BAK may be a detergent, a quaternary ammonium compound with a broad range of antimicrobial activity. In some embodiments, BAK may be a preservative in the pharmaceutical composition.

In some embodiments, a carrier and/or a solvent of the timolol 0.5%, brimonidine tartrate 0.2%, and dorzolamide 2% pharmaceutical composition may be water. In some embodiments, this water may be sterile water, water for irrigation, water for injection (WFI), or the like.

In some embodiments, the timolol 0.5%, brimonidine tartrate 0.2%, and dorzolamide 2% pharmaceutical composition may be delivered to the eyes of a patient via use eye drops from an eye dropper.

In some embodiments, the timolol 0.5%, brimonidine tartrate 0.2%, and dorzolamide 2% pharmaceutical composition may be used for treating glaucoma in the eye(s).

In some embodiments, compounding the pharmaceutical composition comprising the timolol 0.5%, brimonidine tartrate 0.2%, and dorzolamide 2% may comprise steps of: (step 101) prepping clean work area (e.g., cleaning and/or disinfecting); (step 102) using only sterilized and/or depyrogenated equipment; (step 103) weighing applicable APIs (e.g., timolol maleate, brimonadine tartrate, and dorzolamide) in a powder hood (with the 0.5%, 0.2%, and 2% targets in mind, respectively); (step 104) dissolving weighed out API powders in sterile water (or WFI) (with the 0.5%, 0.2%, and 2% targets in mind, respectively); (step 105) testing and adjusting the pH to a target of 5.8 via use of sodium phosphate monobasic, sodium hydroxide, and pH meter (calibrated); (step 106) qs (“quantity sufficient”) with the sterile water (or WFI) with the 0.5%, 0.2%, and 2% targets in mind, respectively; (step 107) transferring resulting solution to CAI; (step 108) sterile filtering (e.g., using 0.22 micron filter) the resulting solution to yield the pharmaceutical composition comprising the timolol 0.5%, brimonidine tartrate 0.2%, and dorzolamide 2%; (step 109) QA/QC tests, such as bubble point testing, sterility testing, and/or endotoxin testing; (step 110) filling final delivery device, e.g., a sterile ophthalmic dropper bottle (e.g., a “drop-tainer,” “steri-dropper,” or the like); and (step 111) labeling and storage. See e.g., FIG. 1. In some embodiments, the final delivery device, e.g., the sterile ophthalmic dropper bottle, may be light resistant.

For example, and without limiting the scope of the present invention, a 100 mL batch of the pharmaceutical composition comprising the timolol 0.5%, brimonidine tartrate 0.2%, and dorzolamide 2% may comprise: 0.68 grams of timolol maleate; 0.2 grams of brimonadine tartrate; 2.225 grams of dorzolamide HCl; 0.86 grams of sodium phosphate monobasic; 1.00 mL of BAK; and the balance of sterile water for irrigation (or WFI).

In some embodiments, the pharmaceutical composition may comprise timolol 0.5%, brimonidine tartrate 0.2%, dorzolamide 2%, sodium phosphate, BAK, sodium hydroxide, and water; wherein that pharmaceutical composition may have a pH of 5.8.

Discussion of Timolol 0.5%, Brimonidine Tartrate 0.2%, Dorzolamide 2%, and Latanoprost 0.005%

In some embodiments, a pharmaceutical composition may comprise at least four active ingredients (APIs), timolol maleate (timolol), brimonidine tartrate, dorzolamide, and latanoprost.

In some embodiments, the timolol may be as discussed above.

In some embodiments, the brimonidine tartrate may be as discussed above.

In some embodiments, the dorzolamide may be as discussed above.

In some embodiments, the latanoprost may be as discussed above.

In some embodiments, this pharmaceutical composition may also comprise sodium chloride for purposes of yielding an isotonic, buffered, aqueous solution.

In some embodiments, this pharmaceutical composition may also comprise sodium chloride and/or sodium hydroxide (NaOH) for purposes of adjusting pH of the resulting pharmaceutical composition to a target pH of 5.8 to 5.9). In some embodiments, HCl may also have been used to adjust pH.

In some embodiments, this pharmaceutical composition may comprise BAK, in addition to the timolol maleate, the brimonidine tartrate, the dorzolamide, and the latanoprost. In some embodiments, BAK may be a detergent, a quaternary ammonium compound with a broad range of antimicrobial activity. In some embodiments, BAK may be a preservative in the pharmaceutical composition.

In some embodiments, a carrier and/or a solvent of the timolol 0.5%, brimonidine tartrate 0.2%, dorzolamide 2%, and latanoprost 0.005% pharmaceutical composition may be water. In some embodiments, this water may be sterile water, water for irrigation, water for injection (WFI), or the like.

In some embodiments, the timolol 0.5%, brimonidine tartrate 0.2%, dorzolamide 2%, and latanoprost 0.005% pharmaceutical composition may be delivered to the eyes of a patient via use eye drops from an eye dropper. In some embodiments, a 10 mL ophthalmic dropper bottle may be used to receive a fill volume of 3.5 mL of this prepared pharmaceutical composition.

In some embodiments, the timolol 0.5%, brimonidine tartrate 0.2%, dorzolamide 2%, and latanoprost 0.005% pharmaceutical composition may be used for treating glaucoma in the eye(s).

In some embodiments, compounding the pharmaceutical composition comprising the timolol 0.5%, brimonidine tartrate 0.2%, dorzolamide 2%, and latanoprost 0.005% may comprise steps of: (step 101) prepping clean work area (e.g., cleaning and/or disinfecting); (step 102) using only sterilized and/or depyrogenated equipment; (step 103) weighing applicable APIs (e.g., timolol maleate, brimonadine tartrate, dorzolamide, and latanoprost) in a powder hood (with the 0.5%, 0.2%, 2%, and 0.005% targets in mind, respectively); (step 104) dissolving weighed out API powders in sterile water (or WFI) (with the 0.5%, 0.2%, 2%, and 0.005% targets in mind, respectively); (step 105) testing and adjusting the pH to a target of 5.8 (of pH 5.8 to 5.9) via use of sodium hydroxide, and pH meter (calibrated); (step 106) qs (“quantity sufficient”) with the sterile water (or WFI) with the 0.5%, 0.2%, 2%, and 0.005% targets in mind, respectively; (step 107) transferring resulting solution to CAI; (step 108) sterile filtering (e.g., using 0.22 micron filter) the resulting solution to yield the pharmaceutical composition comprising the timolol 0.5%, brimonidine tartrate 0.2%, dorzolamide 2%, and latanoprost 0.005%; (step 109) QA/QC tests, such as, clarity, appearance, bubble point testing, sterility testing, and/or endotoxin testing; (step 110) filling final delivery device, e.g., a sterile ophthalmic dropper bottle (e.g., a “drop-tainer,” “steri-dropper,” or the like); and (step 111) labeling and storage. See e.g., FIG. 1. In some embodiments, the final delivery device, e.g., the sterile ophthalmic dropper bottle, may be light resistant.

For example, and without limiting the scope of the present invention, a 100 mL batch of the pharmaceutical composition comprising the timolol 0.5%, brimonidine tartrate 0.2%, dorzolamide 2%, and latanoprost 0.005% may comprise: 0.68 grams of timolol maleate; 0.2 grams of brimonidine tartrate; 2.225 grams of dorzolamide HCl; 0.515 grams of latanoprost stock (at 10 mg/g); 0.42 grams of sodium chloride; 1.00 mL of BAK 1%; and the balance of sterile water (or WFI).

In some embodiments, it may be important not to exceed a pH of 6 with this pharmaceutical composition, as higher pH's may cause undesired precipitation.

In some embodiments, the pharmaceutical composition may comprise timolol 0.5%, brimonidine tartrate 0.2%, dorzolamide 2%, latanoprost 0.005%, sodium chloride, BAK, sodium hydroxide, and water; wherein that pharmaceutical composition may have a pH of 5.8 (or a pH of 5.8 to 5.9). In some embodiments, this pharmaceutical composition may have a beyond-use date of at least 180 days. This pharmaceutical composition may be stored refrigerated. In some embodiments, this pharmaceutical composition may be slightly opaque and slightly yellowish in appearance.

In some embodiments, this pharmaceutical composition with the at least three active ingredients (APIs), may have increased efficacy, improved drug delivery, better patient compliance, and have cost savings for patients (e.g., purchasing less medication containers/delivery devices), as compared against pharmaceutical compositions with only one or two active ingredients (APIs).

Discussion of Moxifloxacin HCL 0.5%

In some embodiments, a pharmaceutical composition may comprise at least one active ingredient (API), moxifloxacin HCl. In some embodiments, this pharmaceutical composition may comprise moxifloxacin HCl at 0.5% (weight per volume). In some embodiments, this pharmaceutical composition may be preservative free; which may result in increased efficacy as compared to pharmaceutical compositions with preservatives.

In some embodiments, moxifloxacin HCl may be known as moxifloxacin or moxifloxacin hydrochloride. In some embodiments, moxifloxacin HCl may be a synthetic fluoroquinolone antibacterial agent. In some embodiments, moxifloxacin may be used in an ophthalmic solution. In some embodiments, moxifloxacin may be used for the treatment of bacterial conjunctivitis (i.e., pink eye). Mechanism of action of moxifloxacin HCl may be through inhibition of DNA gyrase and topoisomerase IV which may be required for some bacterial DNA replication, transcription, repair, and/or recombination.

In some embodiments, the moxifloxacin HCl may be present in the pharmaceutical composition at 0.5%; that is, each mL of the pharmaceutical composition may contain 5 mg of moxifloxacin HCl.

In some embodiments, this pharmaceutical composition may also comprise sodium chloride for purposes of yielding an isotonic, buffered, aqueous solution.

In some embodiments, this pharmaceutical composition may also comprise sodium chloride, sodium hydroxide, and/or hydrochloric acid (HCl) for purposes of adjusting pH of the resulting pharmaceutical composition to a final target pH of 7.2.

In some embodiments, a carrier and/or a solvent of the moxifloxacin HCl 0.5% pharmaceutical composition may be water. In some embodiments, this water may be sterile water, water for injection (WFI), or the like.

In some embodiments, the moxifloxacin HCl 0.5% pharmaceutical composition may be delivered to the eyes of a patient via use of an injection, as in an intra-cameral injection.

In some embodiments, the moxifloxacin HCl 0.5% pharmaceutical composition may be used for post-op care of an eye after cataract surgery of that eye. Such use of the moxifloxacin HCl 0.5%, post-op, may minimize post-op infections; and/or may improve recovery from the cataract surgery, both in terms of speed of recovery quality of vision improvement.

In some embodiments, compounding the pharmaceutical composition comprising the moxifloxacin HCl 0.5% may comprise steps of: (step 101) prepping clean work area (e.g., cleaning and/or disinfecting); (step 102) using only sterilized and/or depyrogenated equipment; (step 103) weighing applicable API (e.g., moxifloxacin HCl) in a powder hood (with the 0.5% target in mind); (step 104) dissolving weighed out API powder in sterile water (or WFI) (with the 0.5% target in mind); (step 105) testing and adjusting the pH to a final target of 7.2 via use of HCl, sodium hydroxide, sodium chloride, and a pH meter (calibrated); (step 106) qs (“quantity sufficient”) with the sterile water (or WFI) with the 0.5% target in mind; (step 107) transferring resulting solution to a CAI; (step 108) sterile filtering (e.g., using a 0.22 micron filter) the resulting solution to yield the pharmaceutical composition comprising the moxifloxacin HCl 0.5%; (step 109) QA/QC tests, such as bubble point testing, sterility testing, and/or endotoxin testing; (step 110) filling final container, e.g., a sterile vial; and (step 111) labeling and storage. See e.g., FIG. 1. In some embodiments, step 104 of dissolving weighed out API powder in sterile water (or WFI), i.e., of dissolving moxifloxacin HCl powder into water, may be facilitated by adding some HCl (e.g., adding 5 mL of HCl 0.1 N per a 100 mL batch) and stirring for about 20 minutes, at room temperature. In some embodiments, a 2 mL sterile vial may be used as the final container. In some embodiments, a 2 mL sterile vial may be filled to 1 mL with the pharmaceutical composition.

For example, and without limiting the scope of the present invention, a 100 mL batch of the pharmaceutical composition comprising the moxifloxacin HCl 0.5% may comprise: 0.523 grams of moxifloxacin HCl; 0.812 grams of sodium chloride; and the balance of sterile water (or WFI); with negligible amounts of HCl and/or sodium hydroxide for pH adjustments to a final target value of 7.2.

Discussion of Dexamethasone PO4 0.1%, Moxifloxacin HCL 0.5%

In some embodiments, a pharmaceutical composition may comprise at least two active ingredients (APIs), dexamethasone PO₄ (phosphate) and moxifloxacin HCl.

In some embodiments, dexamethasone may be known as dexamethasone PO₄, dexamethasone phosphate, dexamethasone sodium PO₄, dexamethasone sodium phosphate, dexamethasone NaPO₄. In some embodiments, dexamethasone PO₄ may be a type of corticosteroid. In some embodiments, dexamethasone PO₄ may be a synthetic glucocorticoid. In some embodiments, dexamethasone PO₄ may be indicated for treatment of inflammation. In some embodiments, dexamethasone may have anti-inflammatory and immunosuppressant effects. In some embodiments, the anti-inflammatory properties of dexamethasone may be useful in post-op care of an eye following cataract surgery of that eye. Mechanism of action of dexamethasone PO₄ may involve inhibition of phospholipase A2, inhibitory proteins, and/or lipocortins which modulate prostaglandins and leukotrienes.

In some embodiments, the dexamethasone PO₄ may be present in the pharmaceutical composition at 0.1%; that is, each mL of the pharmaceutical composition may contain 1 mg of the dexamethasone PO₄.

In some embodiments, the moxifloxacin HCl may be as discussed above.

In some embodiments, this pharmaceutical composition may also comprise sodium chloride (NaCl) for purposes of yielding an isotonic, buffered, aqueous solution.

In some embodiments, this pharmaceutical composition may also comprise sodium chloride, sodium hydroxide (NaOH), and/or hydrochloric acid (HCl) for purposes of adjusting pH of the resulting pharmaceutical composition to a final target pH of 7 to 7.2.

In some embodiments, a carrier and/or a solvent of the dexamethasone PO₄ 0.1%, moxifloxacin HCl 0.5% pharmaceutical composition may be water. In some embodiments, this water may be sterile water, water for injection (WFI), or the like.

In some embodiments, the dexamethasone PO₄ 0.1%, moxifloxacin HCl 0.5% pharmaceutical composition may be delivered to the eyes of a patient via use of an injection, as in an intra-cameral injection. In some embodiments, a sterile and/or depyrogenated 2 mL glass vial may receive a fill volume of 1 mL of this pharmaceutical composition.

In some embodiments, the dexamethasone PO₄ 0.1%, moxifloxacin HCl 0.5% pharmaceutical composition may be used for post-op care of an eye after cataract surgery of that eye. Such use of the dexamethasone PO₄ 0.1%, moxifloxacin HCl 0.5%, post-op, may minimize post-op infections; and/or may improve recovery from the cataract surgery, both in terms of speed of recovery quality of vision improvement.

In some embodiments, compounding the pharmaceutical composition comprising the dexamethasone PO₄ 0.1%, the moxifloxacin HCl 0.5% may comprise steps of: (step 101) prepping clean work area (e.g., cleaning and/or disinfecting); (step 102) using only sterilized and/or depyrogenated equipment; (step 103) weighing applicable APIs (e.g., the dexamethasone NaPO₄ and the moxifloxacin HCl) in a powder hood (with the 0.1% and the 0.5% targets in mind); (step 104) dissolving the weighed out API powders in sterile water (or WFI) (with the 0.1% and the 0.5% targets in mind); (step 105) testing and adjusting the pH to a final target of 7.2 via use of HCl, sodium hydroxide, sodium chloride, and a pH meter (calibrated); (step 106) qs (“quantity sufficient”) with the sterile water (or WFI) with the 0.1% and the 0.5% targets in mind; (step 107) transferring resulting solution to a CAI; (step 108) sterile filtering (e.g., using a 0.22 micron filter) the resulting solution to yield the pharmaceutical composition comprising the dexamethasone PO₄ 0.1%, moxifloxacin HCl 0.5%; (step 109) QA/QC tests, such as bubble point testing, sterility testing, and/or endotoxin testing; (step 110) filling final container, e.g., a sterile vial; and (step 111) labeling and storage. In some embodiments, step 104 of dissolving weighed out API powders in sterile water (or WFI), i.e., of dissolving the moxifloxacin HCl powder into the water, may be facilitated by adding some HCl (e.g., adding 5 mL of HCl 0.1 N per a 100 mL batch) and stirring for about 20 minutes, at room temperature. Which in some embodiments, may then be followed by adjusting the pH to 6.9 using about 20 mL of sodium hydroxide 1%; and then adding in the sodium chloride and dexamethasone sodium phosphate; and then proceeding with step 105 of adjusting the final pH to 7.2. In some embodiments, a 2 mL sterile vial may be used as the final container. In some embodiments, a 2 mL sterile (and/or depyrogenated) vial may be filled to 1 mL with the pharmaceutical composition.

For example, and without limiting the scope of the present invention, a 100 mL batch of the pharmaceutical composition comprising the dexamethasone PO₄ 0.1%, moxifloxacin HCl 0.5% may comprise: 0.523 grams of moxifloxacin HCl; 0.114 grams of dexamethasone sodium PO₄; 0.812 grams of sodium chloride; and the balance of sterile water (or WFI); with negligible amounts of HCl and/or sodium hydroxide for pH adjustments to a final target value of 7.2.

In some embodiments, the pharmaceutical composition may comprise dexamethasone PO₄ 0.1%, moxifloxacin HCl 0.5%, sodium chloride, hydrochloric acid, sodium hydroxide, and water; wherein that pharmaceutical composition may have a pH of 7 to 7.2. In some embodiments, this pharmaceutical composition may be preservative free; which may result in increased efficacy as compared to pharmaceutical compositions with preservatives. In some embodiments, this pharmaceutical composition may have a beyond-use date of at least 180 days. Storage may be room temperature. In some embodiments, this pharmaceutical composition may be clear and yellowish in appearance.

In some embodiments, this pharmaceutical composition with dexamethasone PO₄ 0.1% and moxifloxacin HCl 0.5% may improve patient compliance by reducing a number treatments.

Discussion of Dexamethasone PO₄ 0.1%, Moxifloxacin HCL 0.5%, Ketorolac Tromethamine 0.5%

In some embodiments, a pharmaceutical composition may comprise at least three active ingredients (APIs), dexamethasone PO₄, moxifloxacin HCl, and ketorolac tromethamine.

In some embodiments, the dexamethasone may be as discussed above.

In some embodiments, the moxifloxacin HCl may be as discussed above.

In some embodiments, the ketorolac tromethamine may also be known as ketorolac, ketorolac tromethamine ophthalmic solution, and ketorolac tromethamine injection. In some embodiments, the ketorolac tromethamine may be a non-steroidal anti-inflammatory drug (NSAID), in the family of heterocyclic acetic acid derivatives. In some embodiments, the ketorolac tromethamine may be used as an analgesic. In some embodiments, ketorolac tromethamine may be used to treat inflammation in the eye, at the eye, and/or around the eye. In some embodiments, ketorolac tromethamine may be sued to treat eye inflammation post eye surgery. In some embodiments, ketorolac tromethamine may be used to during eye surgery, during an intraocular ophthalmic procedure, and/or before an intraocular procedure in preparation for that procedure. In some embodiments, the pharmaceutical composition containing ketorolac tromethamine may have a pH of 7.2 to 7.8. Mechanism of action for K may be through inhibition of prostaglandin synthesis secondary to inhibition of COX (cyclooxygenase) production; wherein COX inhibition may be nonselective.

In some embodiments, the ketorolac tromethamine may be present in the pharmaceutical composition at 0.5%; that is, each mL of the pharmaceutical composition may contain 5 mg of the ketorolac tromethamine.

In some embodiments, this pharmaceutical composition may also comprise sodium chloride for purposes of yielding an isotonic, buffered, aqueous solution.

In some embodiments, this pharmaceutical composition may also comprise sodium chloride, sodium hydroxide, and/or hydrochloric acid (HCl) for purposes of adjusting pH of the resulting pharmaceutical composition to a final target pH of 7.0 to 7.2.

In some embodiments, a carrier and/or a solvent of the dexamethasone PO₄ 0.1%, moxifloxacin HCl 0.5%, ketorolac tromethamine 0.5% pharmaceutical composition may be water. In some embodiments, this water may be, sterile water for injection (WFI).

In some embodiments, the dexamethasone PO₄ 0.1%, moxifloxacin HCl 0.5%, ketorolac tromethamine 0.5% pharmaceutical composition may be delivered to the eyes of a patient via use of an injection, as in an intra-cameral injection.

In some embodiments, the dexamethasone PO₄ 0.1%, moxifloxacin HCl 0.5%, ketorolac tromethamine 0.5% pharmaceutical composition may be used for post-op care of an eye after cataract surgery of that eye. Such use of the dexamethasone PO₄ 0.1%, moxifloxacin HCl 0.5%, ketorolac tromethamine 0.5%, post-op, may minimize post-op infections; and/or may improve recovery from the cataract surgery, both in terms of speed of recovery quality of vision improvement.

In some embodiments, compounding the pharmaceutical composition comprising the dexamethasone PO₄ 0.1%, the moxifloxacin HCl 0.5%, ketorolac tromethamine 0.5% may comprise steps of: (step 101) prepping clean work area (e.g., cleaning and/or disinfecting); (step 102) using only sterilized and/or depyrogenated equipment; (step 103) weighing applicable APIs (e.g., moxifloxacin HCl, the dexamethasone NaPO₄ and the ketorolac tromethamine) in a powder hood (with the 0.5%, 0.1% and the 0.5% targets in mind, respectively); (step 104) dissolving the weighed out API powders in sterile water (or WFI) (with the 0.5%, 0.1% and the 0.5% targets in mind, respectively); (step 105) testing and adjusting the pH to a final target of 7.0 to 7.2 via use of HCl, sodium hydroxide, sodium chloride, and a pH meter (calibrated); (step 106) qs (“quantity sufficient”) with the sterile water (or WFI) with the 0.5%, 0.1% and the 0.5% targets in mind, respectively; (step 107) transferring resulting solution to a CAI; (step 108) sterile filtering (e.g., using a 0.22 micron filter) the resulting solution to yield the pharmaceutical composition comprising the dexamethasone PO₄ 0.1%, moxifloxacin HCl 0.5%, ketorolac tromethamine 0.5%; (step 109) QA/QC tests, such as bubble point testing, sterility testing, and/or endotoxin testing; (step 110) filling final container, e.g., a sterile vial; and (step 111) labeling and storage. See e.g., FIG. 1. In some embodiments, step 104 of dissolving weighed out API powders in sterile water (or WFI), i.e., of dissolving the moxifloxacin HCl powder into the water, may be facilitated by adding some HCl (e.g., adding 5 mL of HCl 0.1 N per a 100 mL batch) and stirring for about 20 minutes, at room temperature. Which in some embodiments, may then be followed by adjusting the pH to 6.9 using about 20 mL of sodium hydroxide 1%; and then adding in the sodium chloride, the dexamethasone sodium phosphate, and the ketorolac tromethamine; and then proceeding with step 105 of adjusting the final pH to 7.0 to 7.2. In some embodiments, a 2 mL sterile vial may be used as the final container. In some embodiments, a 2 mL sterile vial may be filled to 1 mL with the pharmaceutical composition.

For example, and without limiting the scope of the present invention, a 100 mL batch of the pharmaceutical composition comprising the dexamethasone PO₄ 0.1%, moxifloxacin HCl 0.5%, ketorolac tromethamine 0.5% may comprise: 0.523 grams of moxifloxacin HCl; 0.104 grams of dexamethasone sodium PO₄; 0.5 grams of ketorolac tromethamine; 0.812 grams of sodium chloride; 1 gram of sodium hydroxide pellets; 1 mL of HCl (1% or 0.1N); and the balance of sterile water (or WFI).

In some embodiments, the pharmaceutical composition may comprise dexamethasone PO₄ 0.1%, moxifloxacin HCl 0.5%, ketorolac tromethamine 0.5%, sodium chloride, hydrochloric acid, sodium hydroxide, and water; wherein that pharmaceutical composition may have a pH of 7 to 7.2. In some embodiments, this pharmaceutical composition may be preservative free; which may result in increased efficacy as compared to pharmaceutical compositions with preservatives.

In some embodiments, this pharmaceutical composition with the at least three active ingredients (APIs), may have increased efficacy, improved drug delivery, better patient compliance, and have cost savings for patients, as compared against pharmaceutical compositions with only one or two active ingredients (APIs).

In some embodiments, this pharmaceutical composition with dexamethasone PO₄ 0.1%, moxifloxacin HCl 0.5% and ketorolac tromethamine 0.5% may improve patient compliance by reducing a number treatments.

Discussion of Dexamethasone PO₄ 0.1%, Moxifloxacin HCL 0.5%, Bromfenac 0.07%

In some embodiments, a pharmaceutical composition may comprise at least three active ingredients (APIs), dexamethasone PO₄, moxifloxacin HCl, and bromfenac.

In some embodiments, the dexamethasone may be as discussed above.

In some embodiments, the moxifloxacin HCl may be as discussed above.

In some embodiments, the bromfenac may be known as bromfenac, bromfenac sodium, and bromfenac ophthalmic solution. In some embodiments, the bromfenac may be known as bromfenac sodium sesquihydrate. In some embodiments, the bromfenac may be a non-steroidal anti-inflammatory drug (NSAID). In some embodiments, bromfenac may block prostaglandin synthesis through cyclooxygenase inhibition, demonstrating COX-2 preference with a lesser affinity for COX-1. In some embodiments, the bromfenac may be used as an analgesic. In some embodiments, bromfenac may be used to treat ocular pain. In some embodiments, bromfenac may be used to treat ocular inflammation. In some embodiments, bromfenac may be used to treat promote and/or facilitate post eye surgery healing and/or health.

In some embodiments, the bromfenac may be present in the pharmaceutical composition at 0.07%; that is, each mL of the pharmaceutical composition may contain 0.07 mg of the bromfenac.

In some embodiments, this pharmaceutical composition may also comprise sodium chloride for purposes of yielding an isotonic, buffered, aqueous solution.

In some embodiments, this pharmaceutical composition may also comprise sodium chloride, sodium hydroxide, and/or hydrochloric acid (HCl) for purposes of adjusting pH of the resulting pharmaceutical composition to a final target pH of 7.0 to 7.2.

In some embodiments, a carrier and/or a solvent of the dexamethasone PO₄ 0.1%, moxifloxacin HCl 0.5%, bromfenac 0.07% pharmaceutical composition may be water. In some embodiments, this water may be sterile water, water for injection (WFI), or the like.

In some embodiments, the dexamethasone PO₄ 0.1%, moxifloxacin HCl 0.5%, bromfenac 0.07% pharmaceutical composition may be delivered to the eyes of a patient via use of an injection, as in an intra-cameral injection.

In some embodiments, the dexamethasone PO₄ 0.1%, moxifloxacin HCl 0.5%, bromfenac 0.07% pharmaceutical composition may be used for post-op care of an eye after cataract surgery of that eye. Such use of the dexamethasone PO₄ 0.1%, moxifloxacin HCl 0.5%, bromfenac 0.07%, post-op, may minimize post-op infections; and/or may improve recovery from the cataract surgery, both in terms of speed of recovery quality of vision improvement.

In some embodiments, compounding the pharmaceutical composition comprising the dexamethasone PO₄ 0.1%, the moxifloxacin HCl 0.5%, bromfenac 0.07% may comprise steps of: (step 101) prepping clean work area (e.g., cleaning and/or disinfecting); (step 102) using only sterilized and/or depyrogenated equipment; (step 103) weighing applicable APIs (e.g., moxifloxacin HCl, the dexamethasone NaPO₄ and the bromfenac) in a powder hood (with the 0.5%, 0.1% and the 0.07% targets in mind, respectively); (step 104) dissolving the weighed out API powders in sterile water (or WFI) (with the 0.5%, 0.1% and the 0.07% targets in mind, respectively); (step 105) testing and adjusting the pH to a final target of 7.0 to 7.2 via use of HCl, sodium hydroxide, sodium chloride, and a pH meter (calibrated); (step 106) qs (“quantity sufficient”) with the sterile water (or WFI) with the 0.5%, 0.1% and the 0.07% targets in mind, respectively; (step 107) transferring resulting solution to a CAI; (step 108) sterile filtering (e.g., using a 0.22 micron filter) the resulting solution to yield the pharmaceutical composition comprising the dexamethasone PO₄ 0.1%, moxifloxacin HCl 0.5%, bromfenac 0.07%; (step 109) QA/QC tests, such as bubble point testing, sterility testing, and/or endotoxin testing; (step 110) filling final container, e.g., a sterile vial; and (step 111) labeling and storage. In some embodiments, step 104 of dissolving weighed out API powders in sterile water (or WFI), i.e., of dissolving the moxifloxacin HCl powder into the water, may be facilitated by adding some HCl (e.g., adding 5 mL of HCl 0.1 N per a 100 mL batch) and stirring for about 20 minutes, at room temperature. Which in some embodiments, may then be followed by adjusting the pH to 6.9 using about 20 mL of sodium hydroxide 1%; and then adding in the sodium chloride, the dexamethasone sodium phosphate, and the bromfenac; and then proceeding with step 105 of adjusting the final pH to 7.0 to 7.2. In some embodiments, a 2 mL sterile vial may be used as the final container. In some embodiments, a 2 mL sterile vial may be filled to 1 mL with the pharmaceutical composition.

For example, and without limiting the scope of the present invention, a 100 mL batch of the pharmaceutical composition comprising the dexamethasone PO₄ 0.1%, moxifloxacin HCl 0.5%, bromfenac 0.07% may comprise: 0.523 grams of moxifloxacin HCl; 0.104 grams of dexamethasone sodium PO₄; 0.07 grams of bromfenac; 0.812 grams of sodium chloride; 1 gram of sodium hydroxide pellets; 1 mL of HCl (1% or 0.1N); and the balance of sterile water (or WFI).

In some embodiments, the pharmaceutical composition may comprise dexamethasone PO₄ 0.1%, moxifloxacin HCl 0.5%, bromfenac 0.07%, sodium chloride, hydrochloric acid, sodium hydroxide, and water; wherein that pharmaceutical composition may have a pH of 6.9. In some embodiments, this pharmaceutical composition may be preservative free; which may result in increased efficacy as compared to pharmaceutical compositions with preservatives.

In some embodiments, this pharmaceutical composition with the at least three active ingredients (APIs), may have increased efficacy, improved drug delivery, better patient compliance, and have cost savings for patients, as compared against pharmaceutical compositions with only one or two active ingredients (APIs).

In some embodiments, this pharmaceutical composition with dexamethasone PO₄ 0.1%, moxifloxacin HCl 0.5% and bromfenac 0.07% may improve patient compliance by reducing a number treatments.

Discussion of Prednisolone PO₄ 1%, Gatifloxacin 0.5%

In some embodiments, a pharmaceutical composition may comprise at least two active ingredients (APIs), prednisolone PO₄ and gatifloxacin.

In some embodiments, prednisolone PO₄ may be known as prednisolone, prednisolone phosphate, prednisolone NaPO₄, or prednisolone sodium phosphate. In some embodiments, prednisolone PO₄ may be prepared to meet USP monograph for prednisolone sodium phosphate ophthalmic solution. In some embodiments, prednisolone PO₄ may be an anti-inflammatory steroid. A mechanism for action of prednisolone PO₄ may be inhibition of migration of polymorphonuclear leukocytes and capilla increase reversal. In some embodiments, prednisolone PO₄ may be used for treating inflammation in the eye, at the eye, and/or around the eye via use of eye drops. In some embodiments, prednisolone PO₄ may be used post eye surgery. In some embodiments, the pharmaceutical composition containing prednisolone PO₄ may have a pH from 6.2 to 8.2. In some embodiments, the prednisolone PO₄ may be present in the pharmaceutical composition at 1%; that is each mL (milliliter) of the pharmaceutical composition may contain 11 mg (milligram) of prednisolone PO₄.

In some embodiments, gatifloxacin may also be known as gatifloxacin sesquihydrate and gatifloxacin ophthalmic solution. In some embodiments, gatifloxacin may be an antibiotic of the fourth-generation fluoroquinolone family of antibiotics. Mechanism of action of gatifloxacin may be through inhibition of DNA gyrase and topoisomerase IV which may be needed for some bacterial DNA replication, transcription, repair, and recombination. In some embodiments, the gatifloxacin may work by inhibiting bacterial enzymes, specifically, DNA gyrase and topoisomerase IV. In some embodiments, gatifloxacin may be indicated for treatment of bacterial conjunctivitis. In some embodiments, the gatifloxacin may be used for treating bacterial conjunctivitis in eyes. In some embodiments, the gatifloxacin may be used for treating bacterial infections in eyes, at the eyes, and/or around the eyes. In some embodiments, the gatifloxacin may be used to prevent or minimize bacterial growth in the eyes, at the eyes, and/or around the eyes. In some embodiments, gatifloxacin may be used post eye surgery (e.g., Lasik) to prevent or minimize bacterial growth in the eyes, at the eyes, and/or around the eyes. In some embodiments, the gatifloxacin may be present in the pharmaceutical composition at 0.5%; that is each mL of pharmaceutical composition may contain 5.4 mg of gatifloxacin.

In some embodiments, this pharmaceutical composition may also comprise one or more of boric acid (for tonicity adjustment and/or as a preservative of the final preparation), sodium hydroxide (for pH adjustments, e.g., added as 1% NaOH), and/or hydrochloric acid (HCl) (for pH adjustments, e.g., added as 1% or 0.1 N HCl). In some embodiments, the final target pH of this pharmaceutical composition comprising the prednisolone PO₄ 1% and the gatifloxacin 0.5% may be a pH of 6.5 or 6.8 to 7. In some embodiments, the boric acid may be a weak acid. In some embodiments, the boric acid may have mild antibiotic properties and/or antifungal properties; and thus, act as a preservative. Boric acid solutions may be used to cleanse and/or irrigate eyes (e.g., helping to remove irritants and/or pollutants from the eyes). Boric acid solutions may provide soothing relief to eye irritation. In some embodiments, aqueous solutions of boric acid may help facilitate dissolving of prednisolone sodium PO₄.

In some embodiments, a carrier and/or a solvent of the prednisolone PO₄ 1% and the gatifloxacin 0.5% pharmaceutical composition may be water. In some embodiments, this water may be sterile water, water for irrigation, water for injection (WFI), or the like.

In some embodiments, the prednisolone PO₄ 1% and the gatifloxacin 0.5% pharmaceutical composition may be delivered to the eyes of a patient via use eye drops from an eye dropper. In some embodiments, a 10 mL ophthalmic drop (e.g., droptainer) may receive a fill volume of this pharmaceutical composition of 3.5 mL.

In some embodiments, the prednisolone PO₄ 1% and the gatifloxacin 0.5% pharmaceutical composition may be used for post-op eye care after Lasik and the like eye surgeries that may use lasers to mitigate vision problems.

In some embodiments, compounding the pharmaceutical composition comprising the prednisolone PO₄ 1% and the gatifloxacin 0.5% may comprise steps of: (step 101) prepping clean work area (e.g., cleaning and/or disinfecting); (step 102) using only sterilized and/or depyrogenated equipment; (step 103) weighing applicable APIs (e.g., prednisolone sodium PO₄ and gatifloxacin) in a powder hood (with the 1% and the 0.5% targets in mind, respectively); (step 104) dissolving weighed out API powders in sterile water (or WFI) (with the 1% and the 0.5% targets in mind, respectively); (step 105) testing and adjusting the pH to a final target value of 7 via use of sodium hydroxide, and hydrochloric acid, and pH meter (calibrated); (step 106) qs (“quantity sufficient”) with the sterile water (or WFI) with the 1% and the 0.5% targets in mind, respectively; (step 107) transferring resulting solution to a CAI; (step 108) sterile filtering (e.g., a 0.22 micron filter) the resulting solution to yield the pharmaceutical composition comprising the prednisolone PO₄ 1% and the gatifloxacin 0.5%; (step 109) QA/QC tests, such as bubble point testing, sterility testing, and/or endotoxin testing; (step 110) filling final delivery device, e.g., a sterile ophthalmic dropper bottle (e.g., a “drop-tainer,” “steri-dropper,” or the like); and (step 111) labeling and storage. See e.g., FIG. 1. In some embodiments, the final delivery device, e.g., the sterile ophthalmic dropper bottle, may be light resistant.

In some embodiments, step 104 of dissolving the APIs in the water, the gatifloxacin may be added to acidified water and dissolved prior to adding the prednisolone sodium PO₄ to that solution. In some embodiments, step 104 may entail adding 0.1 N HCl to acidify water, then adding and dissolving the gatifloxacin (e.g., via stirring); then adding in the boric acid, NaOH and the prednisolone; wherein the NaOH are added to adjust the final pH to a target of 7. In some embodiments, gatifloxacin powder (solids) may dissolve in water at a pH of 5 or less; however, once dissolved, gatifloxacin may remain in aqueous solution at pH's above 8.5. In some embodiments, the final target pH of this pharmaceutical composition may be from 6.5 to 7.

For example, and without limiting the scope of the present invention, a 100 mL batch of the pharmaceutical composition comprising the prednisolone PO₄ 1% and the gatifloxacin 0.5% may comprise: 1.186 grams of prednisolone sodium PO₄; 0.54 grams of gatifloxacin; 1.25 grams of boric acid; amounts of sodium hydroxide 1% and/or hydrochloric acid 1% or 0.1 N for pH adjustments; and the balance of sterile water (or WFI).

In some embodiments, the pharmaceutical composition may comprise prednisolone PO₄ 1%, gatifloxacin 0.5%, boric acid, hydrochloric acid, sodium hydroxide, and water; wherein that pharmaceutical composition may have a pH of 6.8 to 7. In some embodiments, a beyond-use date for this pharmaceutical composition may be at least 180 days. And storage may be room temperature. In some embodiments, this pharmaceutical composition may be clear, colorless, and free of visible (with naked eye) particulate matter. In some embodiments, this pharmaceutical composition may be preservative free (aside from the boric acid); which may result in increased efficacy as compared to pharmaceutical compositions with preservatives.

Discussion of Prednisolone PO₄ 1%, Ketorolac Tromethamine 0.5%

In some embodiments, a pharmaceutical composition may comprise at least two active ingredients (APIs), prednisolone PO₄ and ketorolac tromethamine.

In some embodiments, the prednisolone PO₄ may be as discussed above.

In some embodiments, the ketorolac tromethamine may be as discussed above, except in this embodiment, the ketorolac tromethamine may be mixed (compounded) with the prednisolone PO₄.

In some embodiments, this pharmaceutical composition may also comprise one or more of boric acid (e.g., for tonicity adjustment and/or as a preservative of the final preparation), sodium hydroxide (for pH adjustments, e.g., added as 1% NaOH), and/or hydrochloric acid (HCl) (for pH adjustments, e.g., added as 1% or 0.1 N HCl). In some embodiments, the final target pH of this pharmaceutical composition comprising the prednisolone PO₄ 1% and the ketorolac tromethamine 0.5% may be a pH of 7.2 to 7.6. In some embodiments, the boric acid may be a weak acid. In some embodiments, the boric acid may have mild antibiotic properties and/or antifungal properties, allowing the boric acid to function as a preservative. Boric acid solutions may be used to cleanse and/or irrigate eyes (e.g., helping to remove irritants and/or pollutants from the eyes). Boric acid solutions may provide soothing relief to eye irritation. In some embodiments, aqueous solutions of boric acid may help facilitate dissolving of prednisolone sodium PO₄.

In some embodiments, a carrier and/or a solvent of the prednisolone PO₄ 1% and the ketorolac tromethamine 0.5% pharmaceutical composition may be water. In some embodiments, this water may be sterile water, water for irrigation, water for injection (WFI), or the like.

In some embodiments, the prednisolone PO₄ 1% and the ketorolac tromethamine 0.5% pharmaceutical composition may be delivered to the eyes of a patient via use eye drops from an eye dropper. In some embodiments, a 10 mL ophthalmic dropper bottle may receive a fill volume of 4 mL of this prepared pharmaceutical composition.

In some embodiments, the prednisolone PO₄ 1% and the ketorolac tromethamine 0.5% pharmaceutical composition may be used for post-op eye care for the retina after eye surgery.

In some embodiments, compounding the pharmaceutical composition comprising the prednisolone PO₄ 1% and the ketorolac tromethamine 0.5% may comprise steps of: (step 101) prepping clean work area (e.g., cleaning and/or disinfecting); (step 102) using only sterilized and/or depyrogenated equipment; (step 103) weighing applicable APIs (e.g., prednisolone sodium PO₄ and ketorolac tromethamine) in a powder hood or the like (with the 1% and the 0.5% targets in mind, respectively); (step 104) dissolving weighed out API powders in sterile water (or WFI) (with the 1% and the 0.5% targets in mind, respectively); (step 105) testing and adjusting the pH to a final target value of 7.2 to 7.6 via use of sodium hydroxide, and hydrochloric acid, and pH meter (calibrated); (step 106) qs (“quantity sufficient”) with the sterile water (or WFI) with the 1% and the 0.5% targets in mind, respectively; (step 107) transferring resulting solution to a CAI; (step 108) sterile filtering (e.g., a 0.22 micron filter) the resulting solution to yield the pharmaceutical composition comprising the prednisolone PO₄ 1% and the ketorolac tromethamine 0.5%; (step 109) QA/QC tests, such as clarity, appearance, bubble point testing, sterility testing, and/or endotoxin testing; (step 110) filling final delivery device, e.g., a sterile ophthalmic dropper bottle (e.g., a “drop-tainer,” “steri-dropper,” or the like); and (step 111) labeling and storage. See e.g., FIG. 1. In some embodiments, the final delivery device, e.g., the sterile ophthalmic dropper bottle, may be light resistant.

For example, and without limiting the scope of the present invention, a 100 mL batch of the pharmaceutical composition comprising the prednisolone PO₄ 1% and the ketorolac tromethamine 0.5% may comprise: 1.186 grams of prednisolone sodium PO₄; 0.50 grams of the ketorolac tromethamine; 1.27 grams of boric acid; amounts of sodium hydroxide 1% and/or hydrochloric acid 1% or 0.1 N for pH adjustments; and the balance of sterile water (or WFI).

In some embodiments, the pharmaceutical composition may comprise prednisolone PO₄ 1%, ketorolac tromethamine 0.5%, boric acid, hydrochloric acid, sodium hydroxide, and water; wherein that pharmaceutical composition may have a pH of 7.2 to 7.6 (or pH 7.2 to 7.4). In some embodiments, this pharmaceutical composition may have a beyond-use date of at least 180 days. Storage may be room temperature. In some embodiments, this pharmaceutical composition may be clear, colorless, and free of visible (with naked eye) particulate matter. In some embodiments, this pharmaceutical composition may be preservative free (aside from the boric acid); which may result in increased efficacy as compared to pharmaceutical compositions with preservatives.

Discussion of Prednisolone PO₄ 1%, Ketorolac Tromethamine 0.5%, Gatifloxacin 0.5%

In some embodiments, a pharmaceutical composition may comprise at least three active ingredients (APIs), prednisolone PO₄, ketorolac tromethamine, and gatifloxacin.

In some embodiments, the prednisolone PO₄ may be as discussed above.

In some embodiments, the ketorolac tromethamine may be as discussed above, except in this embodiment, the ketorolac tromethamine may be mixed (compounded) with the prednisolone PO₄ and with gatifloxacin.

In some embodiments, the gatifloxacin may be as discussed above.

In some embodiments, this pharmaceutical composition may also comprise one or more of boric acid (for tonicity adjustment), sodium hydroxide (for pH adjustment, e.g., added as 5% NaOH), and/or hydrochloric acid (HCl) (for pH adjustment, e.g., added as 1% or 0.1 N HCl). In some embodiments, the final target pH of this pharmaceutical composition comprising the prednisolone PO₄ 1%, the ketorolac tromethamine 0.5%, and the gatifloxacin 0.5% may be a pH of 8.5 or greater. In some embodiments, the boric acid may be a weak acid. In some embodiments, the boric acid may have mild antibiotic properties and/or antifungal properties, such that this boric acid may function as a preservative. Boric acid solutions may be used to cleanse and/or irrigate eyes (e.g., helping to remove irritants and/or pollutants from the eyes). Boric acid solutions may provide soothing relief to eye irritation. In some embodiments, aqueous solutions of boric acid may help facilitate dissolving of prednisolone sodium PO₄.

In some embodiments, a carrier and/or a solvent of the prednisolone PO₄ 1%, the ketorolac tromethamine 0.5%, and the gatifloxacin 0.5% pharmaceutical composition may be water. In some embodiments, this water may be sterile water, water for irrigation, water for injection (WFI), or the like.

In some embodiments, the prednisolone PO₄ 1%, the ketorolac tromethamine 0.5%, and the gatifloxacin 0.5% pharmaceutical composition may be delivered to the eyes of a patient via use eye drops from an eye dropper. In some embodiments, a fill volume of 3.5 mL or 6.5 mL of this pharmaceutical composition may be contained in a 10 mL ophthalmic dropper (e.g., droptainer or the like).

In some embodiments, the prednisolone PO₄ 1%, the ketorolac tromethamine 0.5%, and the gatifloxacin 0.5% pharmaceutical composition may be used for post-op eye care after cataract surgery.

In some embodiments, compounding the pharmaceutical composition comprising the prednisolone PO₄ 1%, the ketorolac tromethamine 0.5%, and the gatifloxacin 0.5% may comprise steps of: (step 101) prepping clean work area (e.g., cleaning and/or disinfecting); (step 102) using only sterilized and/or depyrogenated equipment; (step 103) weighing applicable APIs (e.g., prednisolone sodium PO₄, ketorolac tromethamine, and gatifloxacin) in a powder hood (with the 1%, the 0.5%, and the 0.5% targets in mind, respectively); (step 104) dissolving weighed out API powders in sterile water (or WFI) (with the 1%, the 0.5%, and the 0.5% targets in mind, respectively); (step 105) testing and adjusting the pH to a final target value of 8.5 or greater via use of sodium hydroxide, and hydrochloric acid, and pH meter (calibrated); (step 106) qs (“quantity sufficient”) with the sterile water (or WFI) with the 1%, the 0.5%, and the 0.5% targets in mind, respectively; (step 107) transferring resulting solution to a CAI; (step 108) sterile filtering (e.g., a 0.22 micron filter) the resulting solution to yield the pharmaceutical composition comprising the prednisolone PO₄ 1%, the ketorolac tromethamine 0.5%, and the gatifloxacin 0.5%; (step 109) QA/QC tests, such as bubble point testing, sterility testing, and/or endotoxin testing; (step 110) filling final delivery device, e.g., a sterile ophthalmic dropper bottle (e.g., a “drop-tainer,” “steri-dropper,” or the like); and (step 111) labeling and storage. See e.g., FIG. 1. In some embodiments, the final delivery device, e.g., the sterile ophthalmic dropper bottle, may be light resistant. In some embodiments, the final delivery device may have a total volume of 15 mL with a fill volume of 6.5 mL. In some embodiments, the final delivery device may be stored at room temperature.

In some embodiments, step 104 of dissolving the APIs in the water, the gatifloxacin may be added to acidified water and dissolved prior to adding of the dissolved ketorolac tromethamine and the dissolved prednisolone sodium PO₄. In some embodiments, step 104 may entail adding 0.1 N HCl to acidify water, then adding and dissolving the gatifloxacin (e.g., via stirring); and then adding NaOH to bring the pH up to 8.5. In some embodiments, gatifloxacin powder (solids) may dissolve in water at a pH of 5 or less; however, once dissolved, gatifloxacin may remain in aqueous solution at pH's above 8.5. In some embodiments, the ketorolac tromethamine may be dissolved separately (separately from dissolving the other APIs) in a volume of water (no need to acidify). In some embodiments, the prednisolone PO₄ may be dissolved separately (separately from dissolving the other APIs) in a volume of water with the boric acid. In some embodiments, once the gatifloxacin has been dissolved and its pH brought up to 8.5; then the dissolved ketorolac tromethamine may be slowly added to the solution; then the pH may be tested to check if the pH is at or above 8.5; and if the pH is too low it may be adjusted with NaOH (e.g., NaOH 5%) to the 8.5 or above level; and then the dissolved prednisolone PO₄ with boric acid may be added to the dissolved gatifloxacin with the dissolved ketorolac tromethamine solution, again testing and making sure the pH remains at or above 8.5. A pH below 8.2 or below 8.3 may result in irreversible and undesirable precipitate.

For example, and without limiting the scope of the present invention, a 100 mL batch of the pharmaceutical composition comprising the prednisolone PO₄ 1%, the ketorolac tromethamine 0.5%, and the gatifloxacin 0.5% may comprise: 1.186 grams of prednisolone sodium PO₄; 0.50 grams of the ketorolac tromethamine; 0.54 grams of gatifloxacin; 0.95 grams of boric acid; amounts of sodium hydroxide 5% and/or hydrochloric acid 1% or 0.1 N for pH adjustments; and the balance of sterile water (or WFI).

In some embodiments, the pharmaceutical composition may comprise prednisolone PO₄ 1%, gatifloxacin 0.5%, ketorolac tromethamine 0.5%, boric acid, hydrochloric acid, sodium hydroxide, and water; wherein that pharmaceutical composition may have a pH of 8.5 or above. In some embodiments, a beyond-use date of at least 180 days may be applicable for this pharmaceutical composition. And this pharmaceutical composition may be stored at room temperature. In some embodiments, this pharmaceutical composition may be clear and yellowish in appearance. In some embodiments, this pharmaceutical composition may be preservative free (aside from the boric acid); which may result in increased efficacy as compared to pharmaceutical compositions with preservatives.

In some embodiments, this pharmaceutical composition with the at least three active ingredients (APIs), may have increased efficacy, improved drug delivery, better patient compliance, and have cost savings for patients, as compared against pharmaceutical compositions with only one or two active ingredients (APIs).

Discussion of Prednisolone PO₄ 1%, Bromfenac 0.07%

In some embodiments, a pharmaceutical composition may comprise at least two active ingredients (APIs), prednisolone PO₄ and bromfenac.

In some embodiments, the prednisolone PO₄ may be as discussed above.

In some embodiments, the bromfenac may be as discussed above, except in this embodiment, the bromfenac may be mixed (compounded) with the prednisolone PO₄.

In some embodiments, this pharmaceutical composition may also comprise one or more of boric acid (for tonicity adjustment), sodium hydroxide (for pH adjustment, e.g., added as 1% NaOH), and/or hydrochloric acid (HCl) (for pH adjustment, e.g., added as 1% or 0.1 N HCl). In some embodiments, the final target pH of this pharmaceutical composition comprising the prednisolone PO₄ 1% and the bromfenac 0.07% may be a pH of 6.8 to 7. In some embodiments, the final target pH of this pharmaceutical composition comprising the prednisolone PO₄ 1% and the bromfenac 0.07% may be a pH of 8.3 to 8.5. In some embodiments, the boric acid may be a weak acid. In some embodiments, the boric acid may have mild antibiotic properties and/or antifungal properties, such that the boric acid functions as a preservative. Boric acid solutions may be used to cleanse and/or irrigate eyes (e.g., helping to remove irritants and/or pollutants from the eyes). Boric acid solutions may provide soothing relief to eye irritation. In some embodiments, aqueous solutions of boric acid may help facilitate dissolving of prednisolone sodium PO₄.

In some embodiments, a carrier and/or a solvent of the prednisolone PO₄ 1% and the bromfenac 0.07% pharmaceutical composition may be water. In some embodiments, this water may be sterile water, water for irrigation, water for injection (WFI), or the like.

In some embodiments, the prednisolone PO₄ 1% and the bromfenac 0.07% pharmaceutical composition may be delivered to the eyes of a patient via use eye drops from an eye dropper. In some embodiments, a fill volume of 4 mL of this pharmaceutical composition may be contained within a 10 mL ophthalmic dropper (e.g., droptainer or the like).

In some embodiments, the prednisolone PO₄ 1% and the bromfenac 0.07% pharmaceutical composition may be used for post-op eye care for the retina after eye surgery.

In some embodiments, compounding the pharmaceutical composition comprising the prednisolone PO₄ 1% and the bromfenac 0.07% may comprise steps of: (step 101) prepping clean work area (e.g., cleaning and/or disinfecting); (step 102) using only sterilized and/or depyrogenated equipment; (step 103) weighing applicable APIs (e.g., prednisolone sodium PO₄ and bromfenac) in a powder hood (with the 1% and the 0.07% targets in mind, respectively); (step 104) dissolving weighed out API powders in sterile water (or WFI) (with the 1% and the 0.07% targets in mind, respectively); (step 105) testing and adjusting the pH to a final target value of 6.8 to 7 via use of sodium hydroxide, and hydrochloric acid, and pH meter (calibrated); (step 106) qs (“quantity sufficient”) with the sterile water (or WFI) with the 1% and the 0.07% targets in mind, respectively; (step 107) transferring resulting solution to a CAI; (step 108) sterile filtering (e.g., a 0.22 micron filter) the resulting solution to yield the pharmaceutical composition comprising the prednisolone PO₄ 1% and the bromfenac 0.07%; (step 109) QA/QC tests, such as bubble point testing, sterility testing, and/or endotoxin testing; (step 110) filling final delivery device, e.g., a sterile ophthalmic dropper bottle (e.g., a “drop-tainer,” “steri-dropper,” or the like); and (step 111) labeling and storage. See e.g., FIG. 1. In some embodiments, the final delivery device, e.g., the sterile ophthalmic dropper bottle, may be light resistant. In some embodiments, the final delivery device may be stored at room temperature.

In some embodiments, step 104 of dissolving the APIs, particularly dissolving the prednisolone PO₄, may be facilitated by use of an aqueous solution of boric acid.

For example, and without limiting the scope of the present invention, a 100 mL batch of the pharmaceutical composition comprising the prednisolone PO₄ 1% and the bromfenac 0.07% may comprise: 1.125 grams of prednisolone sodium PO₄; 0.07 grams of the bromfenac; 1.25 grams of boric acid; amounts of sodium hydroxide 1% and/or hydrochloric acid 1% or 0.1 N for pH adjustments; and the balance of sterile water (or WFI).

In some embodiments, the pharmaceutical composition may comprise prednisolone PO₄ 1%, bromfenac 0.07%, boric acid, hydrochloric acid (optionally if needed for pH adjustment), sodium hydroxide, and water; wherein that pharmaceutical composition may have a pH of 6.8 to 7.

In some embodiments, the pharmaceutical composition may comprise prednisolone PO₄ 1%, bromfenac 0.07%, boric acid, hydrochloric acid (optionally if needed for pH adjustment), sodium hydroxide, and water; wherein that pharmaceutical composition may have a pH of 8.3 to 8.5.

In some embodiments, the pharmaceutical composition may comprise prednisolone PO₄ 1%, bromfenac 0.07%, boric acid, hydrochloric acid (optionally if needed for pH adjustment), sodium hydroxide, and water; wherein this pharmaceutical composition may have a beyond-use date of at least 180 days. And this pharmaceutical composition may be stored at room temperature.

In some embodiments, the pharmaceutical composition may comprise prednisolone PO₄ 1%, bromfenac 0.07%, boric acid, hydrochloric acid (optionally if needed for pH adjustment), sodium hydroxide, and water; wherein this pharmaceutical composition may have a beyond-use date of at least 180 days. And this pharmaceutical composition may be stored at room temperature. In some embodiments, this pharmaceutical composition may be clear and yellowish in appearance.

In some embodiments, this pharmaceutical composition comprising the prednisolone PO₄ 1% and the bromfenac 0.07% may be preservative free (aside from the boric acid); which may result in increased efficacy as compared to pharmaceutical compositions with preservatives.

Discussion of Prednisolone PO₄ 1%, Gatifloxacin 0.5%, Bromfenac 0.07%

In some embodiments, a pharmaceutical composition may comprise at least three active ingredients (APIs), prednisolone PO₄, gatifloxacin, and bromfenac.

In some embodiments, the prednisolone PO₄ may be as discussed above.

In some embodiments, the gatifloxacin may be as discussed above, except in this embodiment, the gatifloxacin may be mixed (compounded) with the prednisolone PO₄ and with the bromfenac.

In some embodiments, the bromfenac may be as discussed above, except in this embodiment, the bromfenac may be mixed (compounded) with the prednisolone PO₄ and with the gatifloxacin.

In some embodiments, this pharmaceutical composition may also comprise one or more of boric acid (for osmotic adjustment and/or as a preservative), sodium hydroxide (for pH adjustment, e.g., added as 1% NaOH), and/or hydrochloric acid (HCl) (for pH adjustment, e.g., added as 1% or 0.1 N HCl). In some embodiments, the final target pH of this pharmaceutical composition comprising the prednisolone PO₄ 1%, gatifloxacin 0.5%, and the bromfenac 0.07% may be a pH of 6.8 to 7. In some embodiments, the final target pH of this pharmaceutical composition comprising the prednisolone PO₄ 1%, gatifloxacin 0.5%, and the bromfenac 0.07% may be a pH of 8.3. In some embodiments, the boric acid may be a weak acid. In some embodiments, the boric acid may have mild antibiotic properties and/or antifungal properties, such that this boric acid functions as a preservative. Boric acid solutions may be used to cleanse and/or irrigate eyes (e.g., helping to remove irritants and/or pollutants from the eyes). Boric acid solutions may provide soothing relief to eye irritation. In some embodiments, aqueous solutions of boric acid may help facilitate dissolving of prednisolone sodium PO₄.

In some embodiments, a carrier and/or a solvent of the prednisolone PO₄ 1%, gatifloxacin 0.5%, and the bromfenac 0.07% pharmaceutical composition may be water. In some embodiments, this water may be sterile water, water for irrigation, water for injection (WFI), or the like.

In some embodiments, the prednisolone PO₄ 1%, gatifloxacin 0.5%, and the bromfenac 0.07% pharmaceutical composition may be delivered to the eyes of a patient via use eye drops from an eye dropper. In some embodiments, a fill volume of 3.5 mL or 6.5 mL of this pharmaceutical composition may be contained within a 10 mL ophthalmic dropper (e.g., a droptainer or the like).

In some embodiments, the prednisolone PO₄ 1%, gatifloxacin 0.5%, and the bromfenac 0.07% pharmaceutical composition may be used for post-op eye care after cataract surgery.

In some embodiments, compounding the pharmaceutical composition comprising the prednisolone PO₄ 1%, the gatifloxacin 0.5%, and the bromfenac 0.07% may comprise steps of: (step 101) prepping clean work area (e.g., cleaning and/or disinfecting); (step 102) using only sterilized and/or depyrogenated equipment; (step 103) weighing applicable APIs (e.g., prednisolone sodium PO₄, gatifloxacin, and bromfenac) in a powder hood (with the 1%, 0.5%, and the 0.07% targets in mind, respectively); (step 104) dissolving weighed out API powders in sterile water (or WFI) (with the 1%, 0.5%, and the 0.07% targets in mind, respectively); (step 105) testing and adjusting the pH to a final target value of 6.8 to 7 via use of sodium hydroxide, and hydrochloric acid, and pH meter (calibrated); (step 106) qs (“quantity sufficient”) with the sterile water (or WFI) with the 1%, 0.5%, and the 0.07% targets in mind, respectively; (step 107) transferring resulting solution to a CAI; (step 108) sterile filtering (e.g., a 0.22 micron filter) the resulting solution to yield the pharmaceutical composition comprising the prednisolone PO₄ 1%, the gatifloxacin 0.5%, and the bromfenac 0.07%; (step 109) QA/QC tests, such as bubble point testing, sterility testing, and/or endotoxin testing; (step 110) filling final delivery device, e.g., a sterile ophthalmic dropper bottle (e.g., a “drop-tainer,” “steri-dropper,” or the like); and (step 111) labeling and storage. See e.g., FIG. 1. In some embodiments, the final delivery device, e.g., the sterile ophthalmic dropper bottle, may be light resistant. In some embodiments, the final delivery device may be stored at room temperature.

In some embodiments, step 104 of dissolving the APIs in the water, the gatifloxacin may be added to acidified water and dissolved prior to adding the prednisolone sodium PO₄ to that solution. In some embodiments, step 104 may entail adding 0.1 N HCl to acidify water, then adding and dissolving the gatifloxacin (e.g., via stirring); then adding in the boric acid, NaOH and the prednisolone PO₄; and the bromfenac; the NaOH are added to adjust the final pH to a target of 6.8 to 7. In some embodiments, gatifloxacin powder (solids) may dissolve in water at a pH of 5 or less; however, once dissolved, gatifloxacin may remain in aqueous solution at pH's above 8.5.

In some embodiments, step 104 of dissolving the APIs, particularly dissolving the prednisolone PO₄, may be facilitated by use of an aqueous solution of boric acid.

For example, and without limiting the scope of the present invention, a 100 mL batch of the pharmaceutical composition comprising the prednisolone PO₄ 1%, the gatifloxacin 0.5%, and the bromfenac 0.07% may comprise: 1.186 grams of prednisolone sodium PO₄; 0.54 grams of gatifloxacin; 0.07 grams of the bromfenac; 0.95 IU of boric acid; amounts of sodium hydroxide 1% (e.g., 1 gram of NaOH pellets) and/or hydrochloric acid 1% or 0.1 N (e.g., 1 mL) for pH adjustments; and the balance of sterile water (or WFI).

In some embodiments, the pharmaceutical composition may comprise prednisolone PO₄ 1%, gatifloxacin 0.5%, bromfenac 0.07%, boric acid, hydrochloric acid (optionally if needed for pH adjustment), sodium hydroxide, and water; wherein that pharmaceutical composition may have a pH of 6.8 to 7.

In some embodiments, the pharmaceutical composition may comprise prednisolone PO₄ 1%, gatifloxacin 0.5%, bromfenac 0.07%, boric acid, hydrochloric acid (optionally if needed for pH adjustment), sodium hydroxide, and water; wherein that pharmaceutical composition may have a pH of 8.3. In some embodiments, this pharmaceutical composition may be clear and yellowish in appearance. In some embodiments, this pharmaceutical composition may have a beyond-use date of at least 180 days. And this pharmaceutical composition may be stored at room temperature.

In some embodiments, this pharmaceutical composition may be preservative free (aside from the boric acid); which may result in increased efficacy as compared to pharmaceutical compositions with preservatives.

In some embodiments, this pharmaceutical composition with the at least three active ingredients (APIs), may have increased efficacy, improved drug delivery, better patient compliance, and have cost savings for patients, as compared against pharmaceutical compositions with only one or two active ingredients (APIs).

Discussion of Prostaglandin E2 (PGE2) and ACE

In some embodiments, a pharmaceutical composition may comprise an amniotic cytokine extract (ACE), that may comprise prostaglandin2 (PGE2). In some embodiments, prostaglandin2 may also be known as prostaglandin E2 or as PGE2. In some embodiments, prostaglandin E2 may be a natural prostaglandin source. Prostaglandins may be lipids or lipid like chemicals and may act or function as hormones in vertebrates. In some embodiments, the amniotic cytokine extract (ACE), may comprise natural prostaglandin E2 (PGE2) at 10,000 pg/mL. In some embodiments, the ACE may comprise cytokines, growth factors, and anti-inflammatory molecules. In some embodiments, the ACE may comprise thrombosonndin-1 (TSP-1), WNT4, PGE2, and GDF11. Cytokines, growth factors, and anti-inflammatory molecules in ACE may play a natural role in natural wound healing and tissue growth.

In some embodiments, the pharmaceutical composition that may comprise ACE and/or PGE2, may be used for the treatment of dry eyes. In some embodiments, the ACE and/or PGE2 may promote tear film, restore tear film, increase tear production, and/or reduce ocular inflammation. In some embodiments, the ACE and/or PGE2 may promote and/or facilitate eyelash growth and/or development.

In some embodiments, the pharmaceutical composition comprising the ACE and/or the PGE2, may also comprise BSS (balanced salt solution). In some embodiments, the BSS may be balanced for ocular use. In some embodiments, the BSS may be an intraocular solution. In some embodiments, the BSS may comprise sodium chloride, potassium chloride, calcium chloride dehydrate, magnesium chloride hexahydrate, sodium acetate trihydrate, sodium citrate dihydrate, sodium hydroxide and/or hydrochloric acid (to adjust pH), and water for injection (WFI). In some embodiments, the BSS may have a pH of substantially 7 to 7.5. In some embodiments, the BSS may have an osmolality of substantially 300 mOsm/Kg. In some embodiments, the BSS may be sterile and/or aseptically filled. For example, and without limiting the scope of the present invention, in some embodiments, each mL of BSS may comprise: sodium chloride (NaCl) 0.64%, potassium chloride (KCl) 0.075%, calcium chloride dihydrate (CaCl₂.2H₂O) 0.048%, magnesium chloride hexahydrate (MgCl₂.6H₂O) 0.03%, sodium acetate trihydrate (C₂H₃NaO₂.3H₂O) 0.39%, sodium citrate dihydrate (C₆H₅Na₃O₇.2H₂O) 0.17%, sodium hydroxide and/or hydrochloric acid (to adjust pH), and WFI. Other similar, but slightly different, formulations of BSS may be used as well.

In some embodiments, the pharmaceutical composition comprising the ACE and/or the PGE2, and the BSS, may comprise the (1) ACE and/or the PGE2 and (2) the BSS in equal volumes of the (1) and the (2). For example, and without limiting the scope of the present invention, a 300 mL batch of this pharmaceutical composition may comprise 150 mL of ACE (or 150 mL of PGE2) and 150 mL of BSS.

In some embodiments, the ACE (and/or PGE2) and the BSS pharmaceutical composition may be delivered to the eyes of a patient via use eye drops from an eye dropper. In some embodiments, the ACE (and/or PGE2) and the BSS pharmaceutical composition may be delivered to the eyes of a patient via an intra-cameral injection to the eye.

In some embodiments, the ACE (and/or PGE2) and the BSS pharmaceutical composition may be used for treating dry eyes.

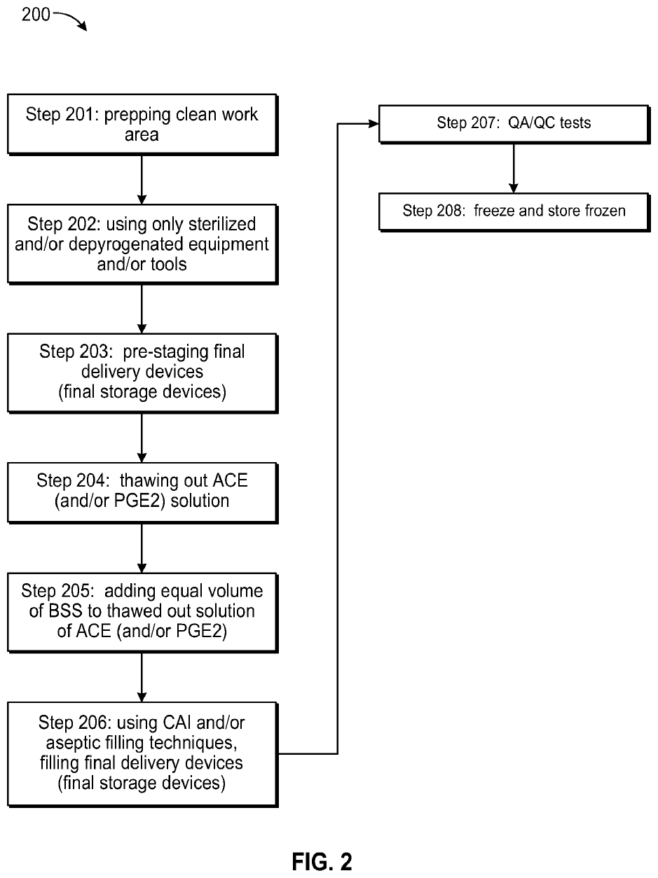

In some embodiments, compounding the pharmaceutical composition comprising the ACE (and/or PGE2) and the BSS may comprise steps of: (step 201) prepping clean work area (e.g., cleaning and/or disinfecting); (step 202) using only sterilized and/or depyrogenated equipment; (step 203) pre-staging the final delivery devices, e.g., sterile ophthalmic dropper bottles (e.g., “drop-tainers,” “steri-droppers,” or the like) and including pre-labeling of these final delivery devices; (step 204) thawing out ACE (and/or PGE2) solution using room temperature water bath; (step 205) adding an equal volume of the BSS to the thawed out solution of ACE (and/or PGE2); (step 206) using CAI and aseptic filling techniques, filling the final delivery devices (e.g., which can include use of a repeater pump); (step 207) QA/QC (quality assurance/quality control) tests, such as sterility testing, pyrogen testing, and/or endotoxin testing; and (step 208) freeze and store frozen at −20 Celsius (Centigrade). See e.g., FIG. 2. In some embodiments, the frozen filled final delivery device should be shipped while frozen (e.g., by use of dry ice) and stored while frozen. In some embodiments, fills of the final delivery device may be at 1 mL or 0.5 mL fill into a 3 mL final delivery device.

In some embodiments, this pharmaceutical composition of ACE (and/or of PGE2) and the BSS, may result in minimal burning, stinging, and/or taste.

In some embodiments, each formulation (e.g., each pharmaceutical composition) disclosed herein, may be compounded and/or filled using aseptic compounding and filling techniques.

In some embodiments, a step of transferring a resulting solution to the CAI (e.g., the compounding aseptic isolator) (e.g., step 107) may be replaced by performing all of the compounding and filling steps in a CAI; and/or performing all of the compounding and filling steps in a clean room; and/or performing all of the compounding and filling steps in a laminar flow hood.

In some embodiments, a step of transferring a resulting solution to the CAI (e.g., the compounding aseptic isolator) (e.g., step 107) may be replaced by performing all or a subset of the compounding and filling steps in a CAI; and/or performing all or a subset of the compounding and filling steps in a clean room; and/or performing all or a subset of the compounding and filling steps in a laminar flow hood.

Discussion of Phenylephrine HCl 1.5%, Lidocaine HCl 1%, Ketorolac Tromethamine 0.3%

In some embodiments, a pharmaceutical composition may comprise at least three active ingredients (APIs): phenylephrine HCl, lidocaine HCl, and ketorolac tromethamine.

In some embodiments, the phenylephrine HCl may be a directly acting sympathomimetic agent (e.g., with α-adrenergic effects) used in the eye as a mydriatic agent (e.g., to dilate the eye's pupil). In the eye, phenylephrine HCl may constrict ophthalmic blood vessels and the radial muscle of the iris.

In some embodiments, the phenylephrine HCl may be present in the pharmaceutical composition at 1.5%; that is, each mL of the pharmaceutical composition may contain 15 mg of the phenylephrine HCl.

In some embodiments, the lidocaine HCl may be a local anesthetic that may reduce pain and/or reduce discomfort during a medical procedure, such as eye surgery, while the lidocaine HCl remains active.

In some embodiments, the lidocaine HCl may be present in the pharmaceutical composition at 1%; that is, each mL of the pharmaceutical composition may contain 10 mg of the lidocaine HCl.

In some embodiments, the ketorolac tromethamine may be a non-steroidal anti-inflammatory drug (NSAID), in the family of heterocyclic acetic acid derivatives. In some embodiments, the ketorolac tromethamine may be used as an analgesic.

In some embodiments, the ketorolac tromethamine may be present in the pharmaceutical composition at 0.3%; that is, each mL of the pharmaceutical composition may contain 3 mg of the ketorolac tromethamine.

In some embodiments, this pharmaceutical composition may also comprise sodium chloride for purposes of yielding an isotonic, buffered, aqueous solution.

In some embodiments, this pharmaceutical composition may also comprise sodium chloride, sodium hydroxide, and/or hydrochloric acid (HCl) for purposes of adjusting pH of the resulting pharmaceutical composition to a final target pH of 6.5.

In some embodiments, a carrier and/or a solvent of the phenylephrine HCl 1.5%, lidocaine HCl 1%, ketorolac tromethamine 0.3% pharmaceutical composition may be water. In some embodiments, this water may be sterile water, water for injection (WFI), or the like.

In some embodiments, the phenylephrine HCl 1.5%, lidocaine HCl 1%, ketorolac tromethamine 0.3% pharmaceutical composition may be delivered to the eyes of a patient via use of an injection, as in an intra-cameral injection.

In some embodiments, the phenylephrine HCl 1.5%, lidocaine HCl 1%, ketorolac tromethamine 0.3% pharmaceutical composition may be used pre-op in preparation for eye surgery, such as, but not limited to, cataract surgery of an eye. In some embodiments, the phenylephrine HCl 1.5%, lidocaine HCl 1%, ketorolac tromethamine 0.3% pharmaceutical composition may be used during eye surgery, such as, but not limited to, cataract surgery of an eye. Such uses of the phenylephrine HCl 1.5%, lidocaine HCl 1%, ketorolac tromethamine 0.3% pharmaceutical composition, may minimize post-op infections; and/or may improve recovery from the eye surgery, both in terms of speed of recovery and quality of vision improvement.

In some embodiments, compounding the pharmaceutical composition comprising the phenylephrine HCl 1.5%, lidocaine HCl 1%, ketorolac tromethamine 0.3% may comprise steps of: (step 101) prepping clean work area (e.g., cleaning and/or disinfecting); (step 102) using only sterilized and/or depyrogenated equipment; (step 103) weighing applicable APIs (e.g., the phenylephrine HCl, the lidocaine HCl, and the ketorolac tromethamine) in a powder hood (with the 1.5%, 1% and the 0.3% targets in mind, respectively); (step 104) dissolving the weighed out API powders in sterile water (or WFI) (with the 1.5%, 1% and the 0.3% targets in mind, respectively); (step 105) testing and adjusting the pH to a final target of 6.5 via use of HCl, sodium hydroxide, sodium chloride, and a pH meter (calibrated); (step 106) qs (“quantity sufficient”) with the sterile water (or WFI) with the 1.5%, 1% and the 0.3% targets in mind, respectively; (step 107) transferring resulting solution to a CAI; (step 108) sterile filtering (e.g., using a 0.22 micron filter) the resulting solution to yield the pharmaceutical composition comprising the phenylephrine HCl 1.5%, the lidocaine HCl 1%, and the ketorolac tromethamine 0.3% as the APIs; (step 109) QA/QC tests, such as bubble point testing, sterility testing, and/or endotoxin testing; (step 110) filling final container, e.g., a sterile vial; and (step 111) labeling and storage. See e.g., FIG. 1. In some embodiments, a 5 mL sterile vial may be used as the final container. In some embodiments, a 5 mL sterile vial may be filled to 4 mL with the pharmaceutical composition of phenylephrine HCl 1.5%, lidocaine HCl 1%, and ketorolac tromethamine 0.3%.

For example, and without limiting the scope of the present invention, a 2050 mL batch of the pharmaceutical composition comprising the phenylephrine HCl 1.5%, lidocaine HCl 1%, and ketorolac tromethamine 0.3% may comprise: 30.75 grams of phenylephrine HCl; 21 grams of lidocaine HCl; 6.15 grams of ketorolac tromethamine; 4.9 grams of sodium chloride; possibly sodium hydroxide pellets and/or HCl (1% or 0.1N) for adjusting final pH to 6.5; and the balance of sterile water (or WFI).

In some embodiments, the pharmaceutical composition may comprise phenylephrine HCl 1.5%, lidocaine HCl 1%, ketorolac tromethamine 0.3%, sodium chloride, water, and/or hydrochloric acid (for pH adjustments), and/or sodium hydroxide (for pH adjustments); wherein that pharmaceutical composition may have a pH of 6.5. In some embodiments, this pharmaceutical composition may be preservative free; which may result in increased efficacy as compared to pharmaceutical compositions with preservatives.

In some embodiments, this pharmaceutical composition with the at least three active ingredients (APIs) (e.g., phenylephrine HCl 1.5%, lidocaine HCl 1%, and ketorolac tromethamine 0.3%), may have increased efficacy, improved drug delivery, better patient compliance, and have cost savings for patients, as compared against pharmaceutical compositions with only one or two active ingredients (APIs).

Discussion of Phenylephrine HCl 1.5%, Lidocaine HCl 1%, Bromfenac 0.01%

In some embodiments, a pharmaceutical composition may comprise at least three active ingredients (APIs): phenylephrine HCl, lidocaine HCl, and bromfenac.

In some embodiments, the phenylephrine HCl may be a directly acting sympathomimetic agent (e.g., with α-adrenergic effects) used in the eye as a mydriatic agent (e.g., to dilate the eye's pupil). In the eye, phenylephrine HCl may constrict ophthalmic blood vessels and the radial muscle of the iris.

In some embodiments, the phenylephrine HCl may be present in the pharmaceutical composition at 1.5%; that is, each mL of the pharmaceutical composition may contain 15 mg of the phenylephrine HCl.

In some embodiments, the lidocaine HCl may be a local anesthetic that may reduce pain and/or reduce discomfort during a medical procedure, such as eye surgery, while the lidocaine HCl remains active.

In some embodiments, the lidocaine HCl may be present in the pharmaceutical composition at 1%; that is, each mL of the pharmaceutical composition may contain 10 mg of the lidocaine HCl.

In some embodiments, the bromfenac may be a non-steroidal anti-inflammatory drug (NSAID). In some embodiments, the bromfenac may be used as an analgesic.

In some embodiments, the bromfenac may be present in the pharmaceutical composition at 0.07%; that is, each mL of the pharmaceutical composition may contain 0.07 mg of the bromfenac.

In some embodiments, the bromfenac may be present in the pharmaceutical composition at 0.01%.

In some embodiments, this pharmaceutical composition may also comprise sodium chloride for purposes of yielding an isotonic, buffered, aqueous solution.

In some embodiments, this pharmaceutical composition may also comprise sodium chloride, sodium hydroxide, and/or hydrochloric acid (HCl) for purposes of adjusting pH of the resulting pharmaceutical composition. In some embodiments, pH may be adjusted to a final target pH of 7.0 to 7.2.

In some embodiments, a carrier and/or a solvent of the phenylephrine HCl 1.5%, lidocaine HCl 1%, bromfenac 0.01% pharmaceutical composition may be sterile water. In some embodiments, this water may be sterile water, water for injection (WFI), or the like.

In some embodiments, the phenylephrine HCl 1.5%, lidocaine HCl 1%, and bromfenac 0.01% pharmaceutical composition may be indicated for pupil dilation and/or pain relief during and/or after eye surgery, such as, but not limited to cataract surgery.

In some embodiments, the phenylephrine HCl 1.5%, lidocaine HCl 1%, bromfenac 0.01% pharmaceutical composition may be delivered to the eye(s) of a patient via use of an injection, as in an intra-cameral injection.

In some embodiments, the phenylephrine HCl 1.5%, lidocaine HCl 1%, bromfenac 0.01% pharmaceutical composition may be used pre-op in preparation for eye surgery, such as, but not limited to, cataract surgery of an eye. In some embodiments, the phenylephrine HCl 1.5%, lidocaine HCl 1%, bromfenac 0.01% pharmaceutical composition may be used during eye surgery, such as, but not limited to, cataract surgery of an eye. Such uses of the phenylephrine HCl 1.5%, lidocaine HCl 1%, bromfenac 0.01% pharmaceutical composition, may minimize post-op infections; and/or may improve recovery from the eye surgery, both in terms of speed of recovery and quality of vision improvement.

In some embodiments, the pharmaceutical composition may comprise phenylephrine HCl 1.5%, lidocaine HCl 1%, bromfenac 0.01%, sodium chloride, sterile water, and/or hydrochloric acid (for pH adjustments), and/or sodium hydroxide (for pH adjustments); wherein that pharmaceutical composition may have a final target pH of 7.0 to 7.2. In some embodiments, this pharmaceutical composition may be preservative free; which may result in increased efficacy as compared to pharmaceutical compositions with preservatives.

In some embodiments, this pharmaceutical composition with the at least three active ingredients (APIs) (e.g., phenylephrine HCl 1.5%, lidocaine HCl 1%, and bromfenac 0.01%), may have increased efficacy, improved drug delivery, better patient compliance, and have cost savings for patients, as compared against pharmaceutical compositions with only one or two active ingredients (APIs).

In the following discussion that addresses a number of embodiments and applications of the present invention, reference is made to the accompanying drawings that form a part thereof, where depictions are made, by way of illustration, of specific embodiments in which the invention may be practiced. It is to be understood that other embodiments may be utilized and changes may be made without departing from the scope of the invention.

FIG. 1 may depict a flow diagram of a method 100; wherein method 100 may comprise the steps for compounding and/or filling a given pharmaceutical composition. In some embodiments, method 100 may comprise steps of: step 101, step 102, step 103, step 104, step 105, step 106, step 107, step 108, step 109, step 110, and step 111. In some embodiments, step 101 may be a step of prepping a clean work area (e.g., cleaning and/or disinfecting). In some embodiments, step 102 may be a step of using only sterilized and/or depyrogenated equipment and/or tools. In some embodiments, step 103 may be a step of weighing out the applicable APIs; e.g., in a powder hood. In some embodiments, step 104 may be a step of dissolving the weighed out API powders in appropriate solution, such as, but not limited to aqueous solutions, including, but not limited to, sterile water (or WFI). In some embodiments, step 105 may be a step of testing (e.g., via a calibrated pH meter) and adjusting (e.g., via adding acids and/or bases) to a pH of a predetermined target value. In some embodiments, step 106 may be a step of qs (“quantity sufficient”) with the appropriate solution (e.g., the sterile water (or the WFI)). In some embodiments, step 107 may be a step of transferring a resulting solution to a compounding aseptic isolator (CAI). In some embodiments, step 108 may be a step of sterile filtering (e.g., a 0.22 micron filter) the resulting solution to yield the given pharmaceutical composition. In some embodiments, step 109 may be a step of QA/QC (quality assurance/quality control) testing, such as bubble point testing, sterility testing, pyrogen testing, endotoxin testing, and/or clarity and/or color comparison testing. In some embodiments, step 110 may be a step of filling a final delivery device(s) (or final storage device or final storage container); such as, but not limited to, sterile ophthalmic dropper bottles (e.g., a “drop-tainers,” “steri-droppers,” or the like), vials, pre-filled syringes, and/or the like. In some embodiments, step 111 may be a step of labeling (e.g., contents, expiration date, lot number, compounding date, and/or the like) and/or storage.

In some embodiments, method 100 may comprise one or more steps from FIG. 1.

FIG. 2 may depict a flow diagram of a method 200; wherein method 200 may comprise the steps for compounding and/or filling a given pharmaceutical composition. In some embodiments, method 200 may comprise steps of: step 201, step 202, step 203, step 204, step 205, step 206, step 207, and step 208.

In some embodiments, step 201 may be a step of prepping the clean work area (e.g., cleaning and/or disinfecting). In some embodiments, step 202 may be a step of using only sterilized and/or depyrogenated equipment and/or tools. In some embodiments, step 203 may be a step of pre-staging the final delivery devices (or final storage devices). In some embodiments, step 203 may also comprise pre-labeling of these final delivery devices (or final storage devices). In some embodiments, step 204 may be a step of thawing out the ACE (and/or the PGE2) solution using room temperature water bath. In some embodiments, step 205 may be a step of adding an equal volume of the BSS to the thawed out solution of the ACE (and/or the PGE2). In some embodiments, step 206 may be a step of using the CAI and/or aseptic filling techniques, filling the final delivery devices (or final storage devices). In some embodiments, step 207 may be a step of QA/QC (quality assurance/quality control) testing, such as, but not limited to, sterility testing, pyrogen testing, endotoxin testing, clarity testing, and/or color testing. In some embodiments, step 208 may be a step of freezing and storing frozen the filled final delivery devices (or final storage devices).

In some embodiments, method 200 may comprise one or more steps from FIG. 2.

Timolol 0.5%, Brimonidine Tartrate 0.2%, Dorzolamide HCl 2%, and Latanoprost 0.005% Study

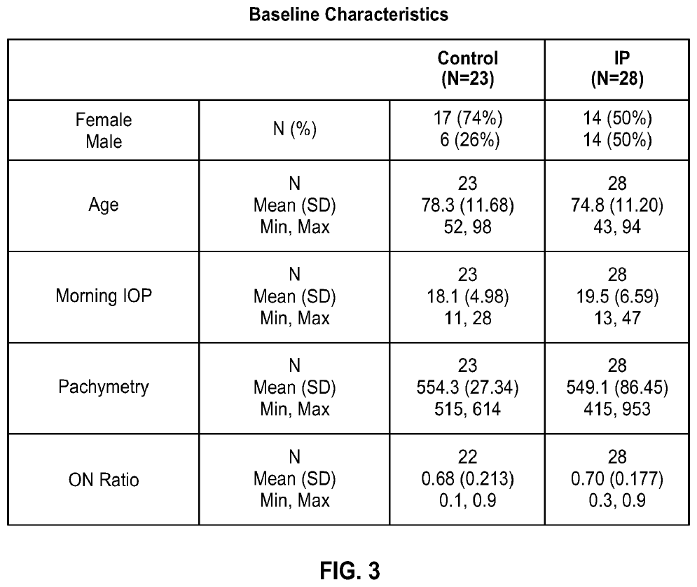

FIG. 3 through FIG. 9 show study results of a multicenter trial of a pharmaceutical composition comprising timolol maleate 0.5%, brimonidine tartrate 0.2%, and dorzolamide HCl 2%; and a pharmaceutical composition comprising timolol maleate 0.5%, brimonidine tartrate 0.2%, dorzolamide HCl 2%, and latanoprost 0.005%; wherein these two pharmaceutical compositions were used to treat glaucoma, specifically, primary open-angle glaucoma (POAG).

In the United States, glaucoma is primarily controlled with topical eye drop therapy with the goal of reducing intraocular pressure (IOP) by 30%. To achieve this goal up to 40% of glaucoma patients will require more than one type topical eye drop medication; i.e., currently the glaucoma patients must use multiple and different eye drop medications to achieve this goal and this presents a patient compliance problem. To increase patient compliance it is desirable to provide only one eye drop delivery device that contains one or more glaucoma medications.

A single bottle of compounded combination glaucoma medication containing multiple molecules (such as, but not limited to timolol maleate, brimonidine tartrate, dorzolamide, and latanoprost in some formulations) may accomplish the necessary 30% IOP reduction while improving patient compliance, simplifying dosing schedule, reducing preservatives, and providing potential cost savings.

A purpose of this study was to demonstrate non-inferiority as well as the safety and efficacy of a compounded combination IOP lowering medication (i.e., the two pharmaceutical compositions used in this study in the IP Group) in the treatment of glaucoma in patients who are currently using three or more separate glaucoma medications (in three or more dropper bottles) who exhibit difficulty with patient compliance and/or have elevated intraocular pressure (IOP).

With respect to the method of this study, this was a randomized multicenter, observer masked study of parallel-groups with primary open-angle glaucoma (POAG). Inclusion criterial required patients to be currently taking at least three IOP different and separate lowering medications. Further with respect to the design of this study, this study utilized randomized 1:1, double masked, prospective, multicenter study of 53 subjects with POAG.

The two pharmaceutical compositions tested in the IP Group were: timolol maleate 0.5%, brimonidine tartrate 0.2%, dorzolamide hydrochloride 2%, with BAK 0.001% qAM; and timolol maleate 0.5%, brimonidine tartrate 0.2%, dorzolamide hydrochloride 2%, latanoprost 0.005%, with BAK 0.001% qhs.

Whereas, in the control group, consisted of patients (subjects) continuing to use their current multiple eye drop therapy of at least three separate and different eye drop glaucoma medications (also known as the multiple drops group).

Subjects were seen for evaluation at baseline and on days 7±2, 30±7, 60±7, and 90±7 following the initial visit and randomization arm was masked.

A primary outcome measurement was IOP change, and secondary measurements included corneal staining, patient-reported symptoms, and visual acuity.

A study eye was defined as the eye with the highest morning IOP score at the screening visit (baseline) and was the primary eye used for analyses. Patients without baseline or any post baseline IOP data were excluded from analyses.

Primary Outcome: Non-inferiority as assessed by upper bound of 2-sided 95% confidence interval for the between-group difference in mean change from baseline at all time points.

Analysis of covariance, ANCOVA, was used to analyze continuous measures, with fixed effects for treatment and investigative site, and baseline as a continuous covariate.

With respect to this study's results, see FIG. 3 through FIG. 9.

FIG. 3 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 3 may depict some baseline characteristics.

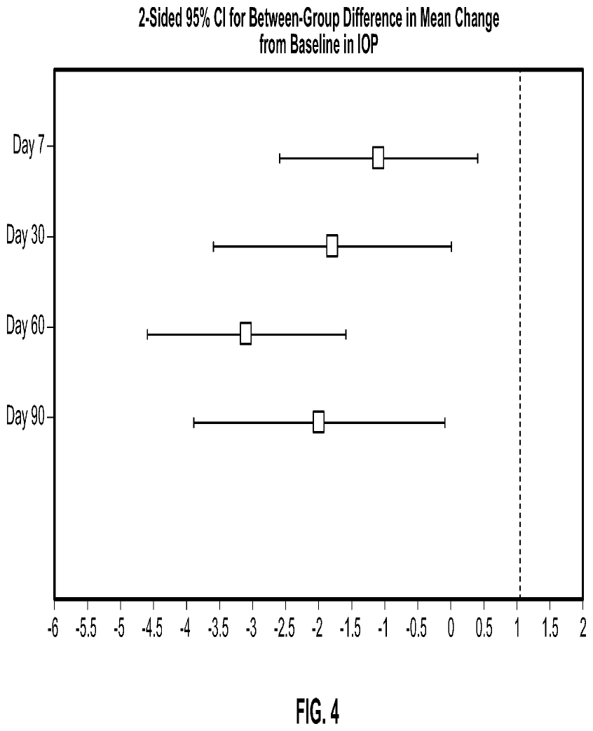

FIG. 4 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 4 may depict a 2-sided 95% confidence interval (CI).

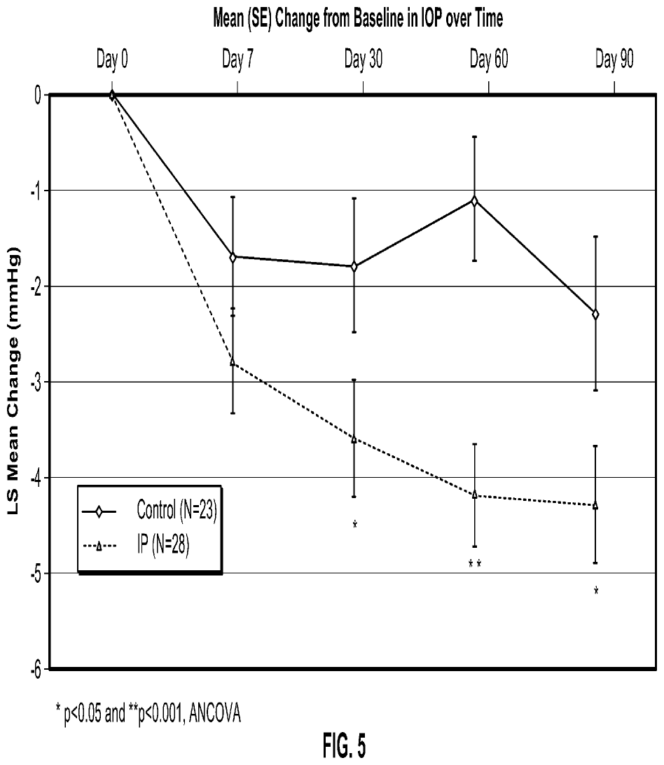

FIG. 5 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 5 may depict mean change from baseline in IOP (intraocular pressure) over time.

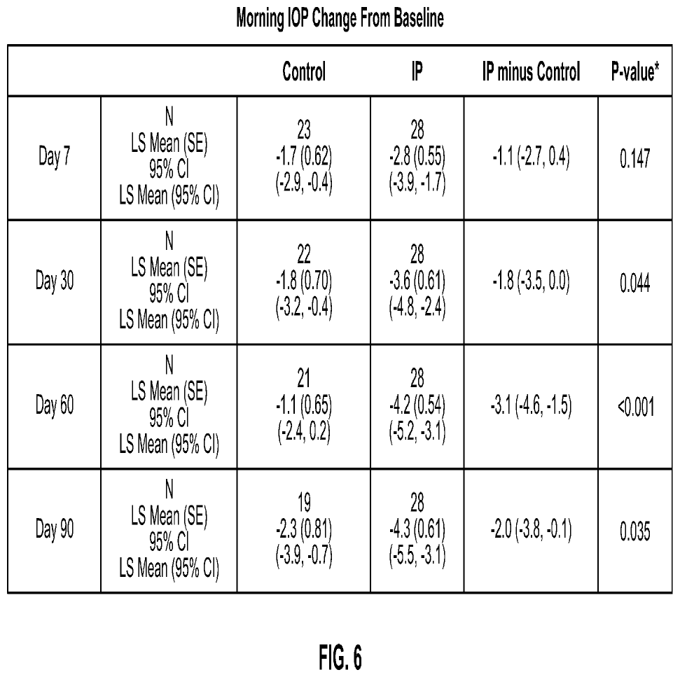

FIG. 6 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 6 may depict morning IOP change from baseline.

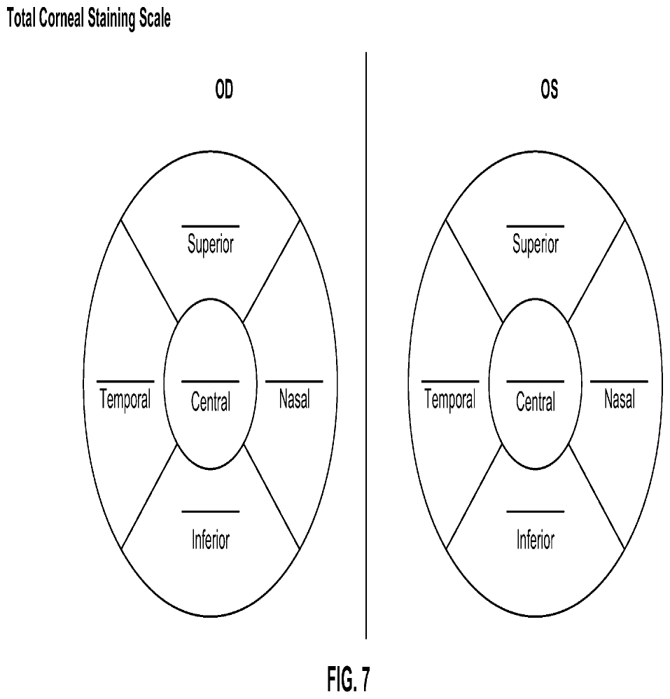

FIG. 7 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 7 may depict total corneal staining scale (TCS) information.

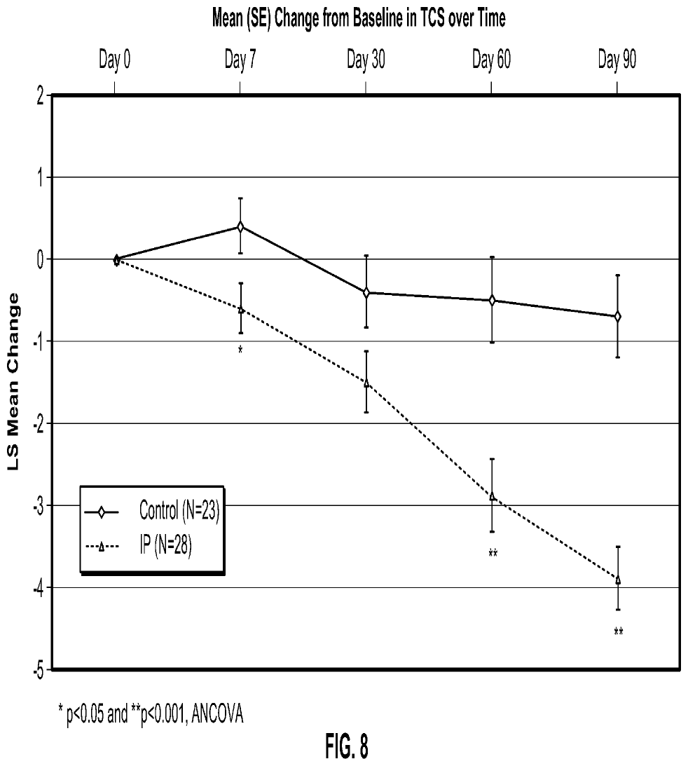

FIG. 8 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 8 may depict mean change from baseline in TCS over time.

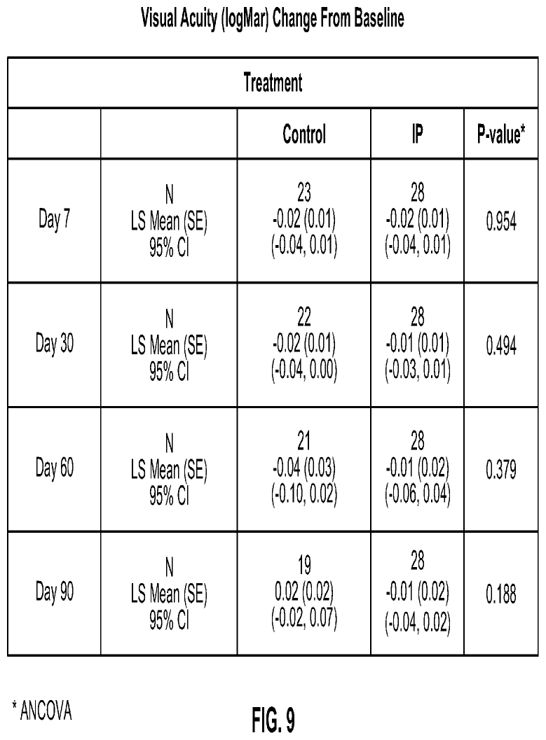

FIG. 9 may depict a table from a study on a pharmaceutical composition comprising timolol maleate 0.5%, brimonidine tartrate 0.2%, and dorzolamide HCl 2%; and on a pharmaceutical composition comprising timolol maleate 0.5%, brimonidine tartrate 0.2%, dorzolamide HCl 2%, and latanoprost 0.005%; wherein FIG. 9 may depict visual acuity change from baseline.

Some of these results may be summarized as follows:
29 patients randomized to IP Group and 24 to Control Group;
2 patients (one in Control and one in IP Group) withdrew consent and discontinued early; 2 with no post-baseline data;
Morning IOP study eye: upper limit of 95% CI was <1.0 mmHg at all time points (days 7, 30, 60 and 90) demonstrating non-inferiority of IP to Control:
   Upper limit of 95% CI<0 at days 30, 60 and 90 indicating superiority of IP;
   Additionally, upper limit <1 for morning fellow eye IOP and both eyes for afternoon IOP at all time points;
Mean decreases from baseline in Total CFS were greater in the IP group and significantly so at days 7, 60 and 90 (study eye and fellow eye);
Changes from baseline in visual acuity were similar between IP and Control (study eye).

With respect to Total Corneal Staining (TCS) Results see FIG. 7 and FIG. 8. Summarizing these TCS results:
82% of subjects assigned to IP and 29% of control subjects showed an improvement from baseline to Day 60;
89% of subjects assigned to IP and 47% of control subjects showed an improvement from baseline to Day 90;
54% of subjects assigned to IP and 5% of control subjects improved at least 4 points from baseline to Day 90;
2 subjects assigned to IP had a 10 point improvement from baseline by Day 90.

Discussion of this Study

In this study, we evaluated a compounded medication containing three or four topical intraocular pressure-lowering molecules (timolol, brimonidine, dorzolamide and latanoprost) (the IP Group) and found that these tested pharmaceutical compositions with either the three or the four intraocular pressure-lowering molecules (timolol, brimonidine, dorzolamide and latanoprost) were not inferior compared to the standard regimen of the Control Group.

At days 30, 60, and 90 of this study, the IP Group showed a greater absolute intraocular pressure reduction than the control. That is, the IP Group showed greater efficacy as compared against the Control Group.

We found no evidence of additional safety problems associated with the use of IP Group versus the standard regimen (Control Group).

Mean differences in corneal fluorescein staining showed that, compared with the standard regiment (Control Group), the IP Group was associated with lower levels of corneal surface lesions. These differences were significant at Days 7, 60, and 90. This may be potentially be explained by the lower levels of preservatives in the IP Group compared to the standard regimen (Control Group).

Conclusions of this Study

In this study, the IP Group was not only not inferior to standard multiple bottle medication regimen (Control Group), but was found to be superior in efficacy. Corneal fluorescein staining showed improvement in the IP Group compared to the group on the standard regimen (Control Group).

There was no evidence of additional safety problems from the compounded IP Group compared to the standard regimen (Control Group).

Inherently there are many benefits to combining three or such four medications (timolol, brimonidine, dorzolamide and latanoprost) into a single eye dropper bottle. These benefits include greater patient compliance, increased efficacy, a lower amount of preservative exposure to the patient, the use of fewer bottles, an easier dosing schedule, and potential cost savings.

Prednisolone PO₄ 1% and Gatifloxacin 0.5% Study

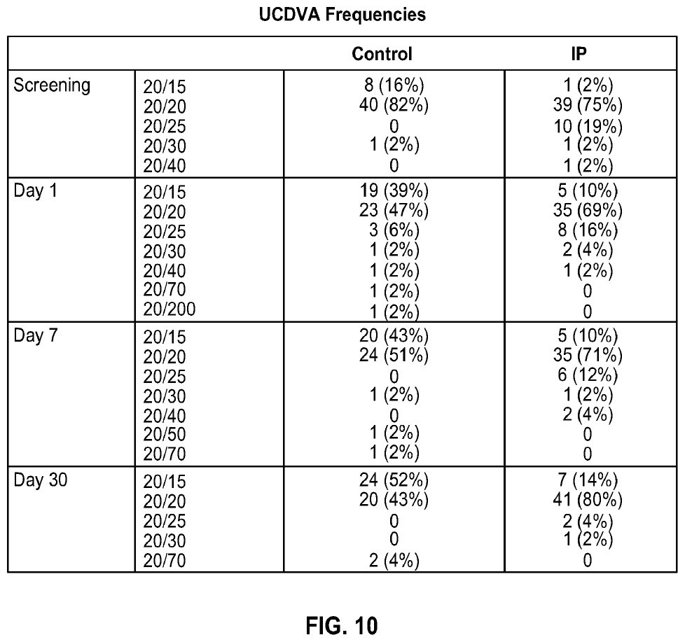

FIG. 10 through FIG. 16 show study results of a multicenter trial of efficacy and tolerability a pharmaceutical composition comprising prednisolone PO₄ (phosphate) 1% and gatifloxacin 0.5%; wherein this pharmaceutical composition was administered as a topical prophylaxis (e.g., as eye drops) after laser vision corrective surgery (e.g., LASIK).

A purpose of this study may have been to demonstrate non-inferiority of using multiple drugs (APIs) in a deliver device (e.g., eye dropper) in the context of post-operative care of an eye after laser eye surgery (e.g., laser-assisted in-situ keratomileusis [LASIK]).

With respect to methodology of this study, this was a randomized 1:1, investigator masked, prospective, multi-center study of 101 refractive surgical eyes who underwent LASIK.

The tested pharmaceutical composition, noted as the IG Group in figures FIG. 10 through FIG. 16, comprised prednisolone phosphate 1%, gatifloxacin 0.5% QID×1 week.

Whereas, the Multiple Drops Group (the Control Group) was two separate and different eye droppers of: (1) prednisolone Acetate 1% QID×1 week; and (2) gatifloxacin Ophthalmic Solution 0.5% QID×1.

Primary Endpoint: Non-inferiority of the prevention of infection and inflammation over 1 month as assessed by upper bound of 2-sided 95% confidence interval for the between-group difference in mean change from baseline. Analysis of covariance, ANCOVA, was used to analyze continuous measures, with fixed effects for treatment and investigative site, and baseline as a continuous covariate. No adjustment made to p-values due to multiple comparisons.

Secondary Endpoint: Refractive Outcomes, and clinical biomicroscopic examination; such as, corneal staining, and change in IOP.

Results of this study may be depicted in figures FIG. 10 through FIG. 16.

FIG. 10 may depict a table from a study on a pharmaceutical composition comprising prednisolone PO₄ (phosphate) 1% and gatifloxacin 0.5%; wherein FIG. 10 may depict UCDVA (uncorrected distance visual acuity) frequencies.

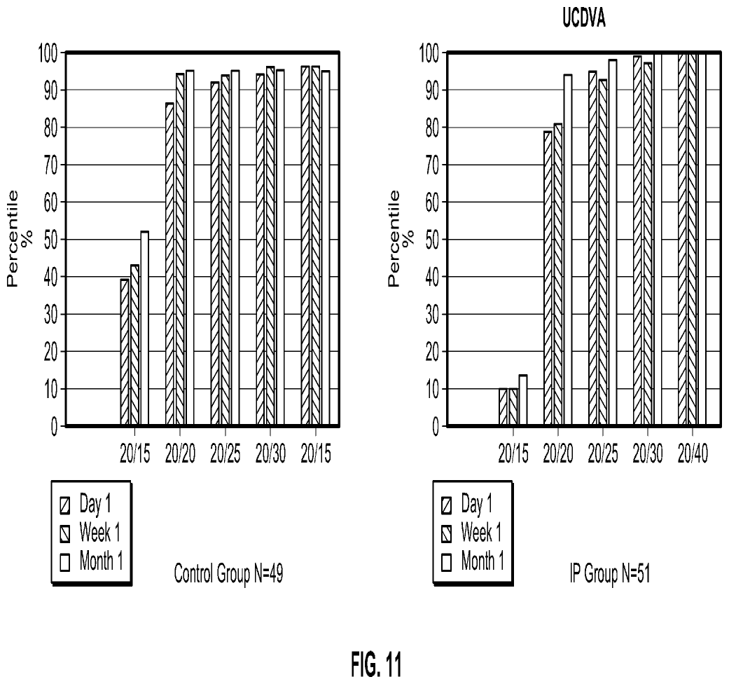

FIG. 11 may depict a table from a study on a pharmaceutical composition comprising prednisolone PO₄ (phosphate) 1% and gatifloxacin 0.5%; wherein FIG. 11 may depict UCDVA (uncorrected distance visual acuity) frequencies.

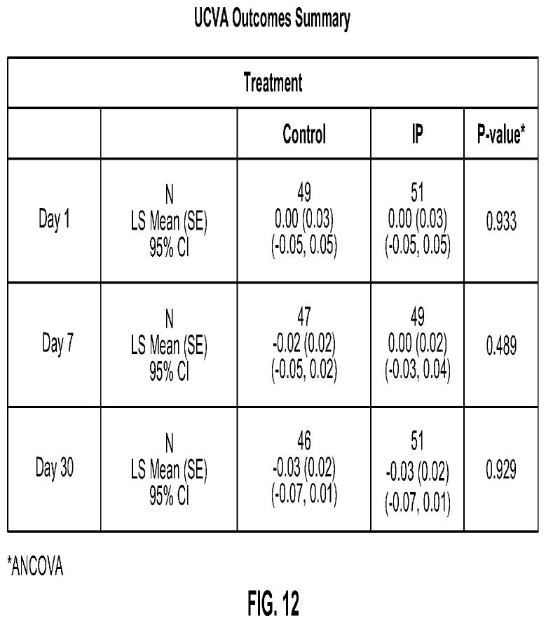

FIG. 12 may depict a table from a study on a pharmaceutical composition comprising prednisolone PO₄ (phosphate) 1% and gatifloxacin 0.5%; wherein FIG. 12 may depict UCVA (uncorrected visual acuity) outcomes summary.

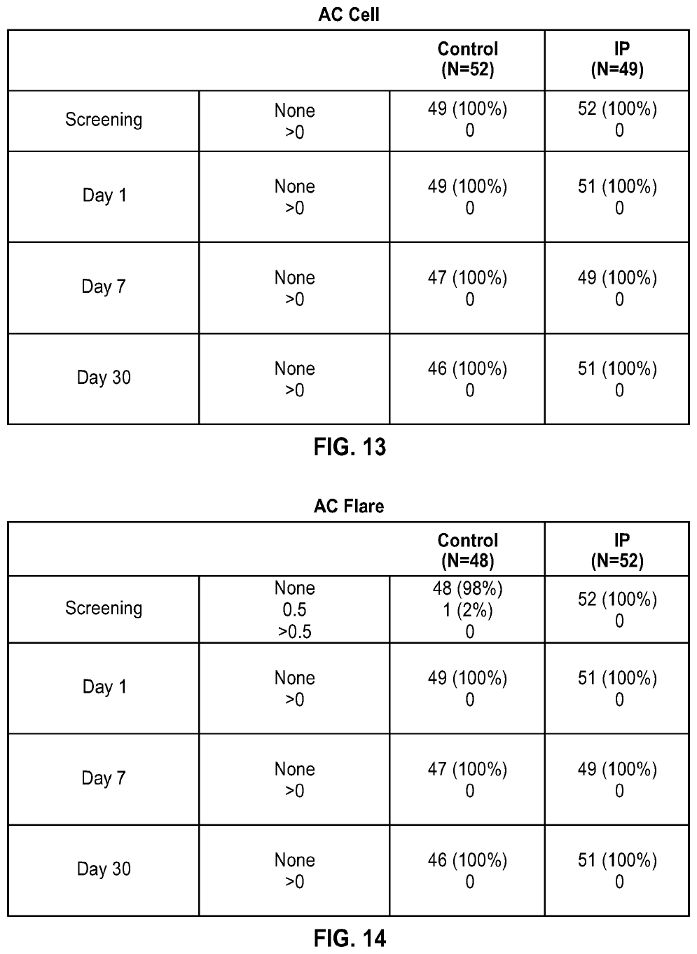

FIG. 13 may depict a table from a study on a pharmaceutical composition comprising prednisolone PO₄ (phosphate) 1% and gatifloxacin 0.5%; wherein FIG. 13 may depict AC Cell results.

FIG. 14 may depict a table from a study on a pharmaceutical composition comprising prednisolone PO₄ (phosphate) 1% and gatifloxacin 0.5%; wherein FIG. 14 may depict AC flare results.

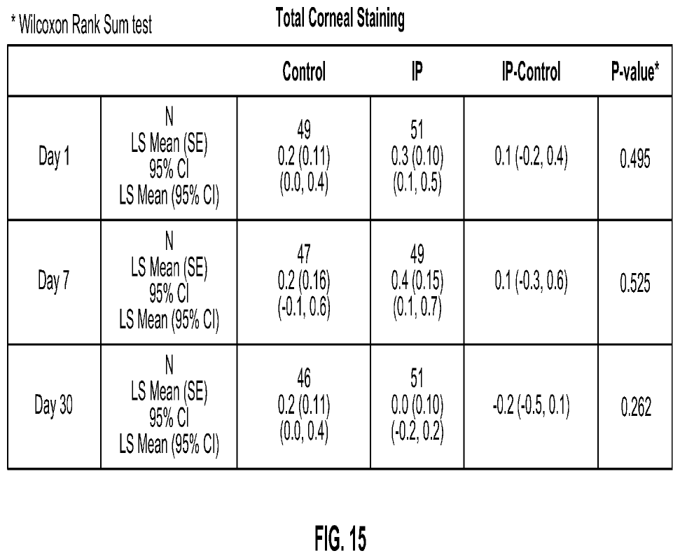

FIG. 15 may depict a table from a study on a pharmaceutical composition comprising prednisolone PO₄ (phosphate) 1% and gatifloxacin 0.5%; wherein FIG. 15 may depict total corneal staining (TCS) results.

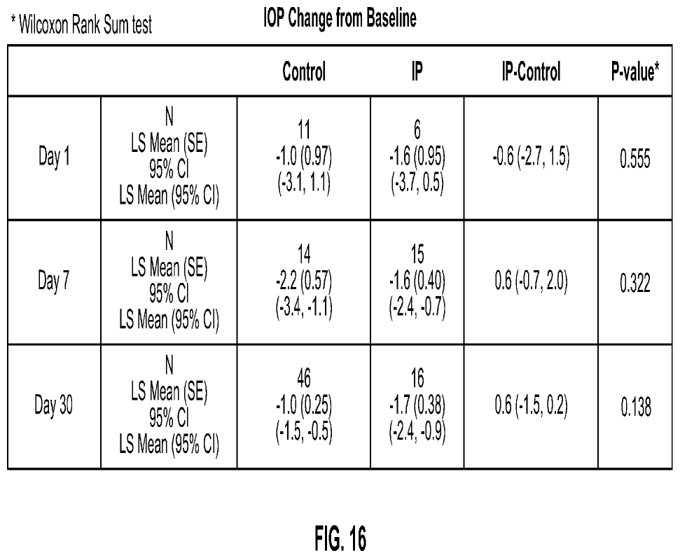

FIG. 16 may depict a table from a study on a pharmaceutical composition comprising prednisolone PO₄ (phosphate) 1% and gatifloxacin 0.5%; wherein FIG. 16 may depict IOP (intraocular pressure) change from baseline.

Some of these results may be summarized as follows:
Study eye was defined as the eye with the highest total corneal staining score at the screening visit (baseline) and was used for analyses;
No significant differences between IP Group and Control Group at any visit as measured by AC cell, AC flare, and change from baseline in IOP, corneal staining, and visual acuity in the study eye;
Corneal Staining: upper limit of 95% CI was <0.2 at Day 30 demonstrating non-inferiority of IP Group to Control Group on this measure;
Mean decreases from baseline to Day 30 in Corneal Staining and IOP were greater in the IP Group, demonstrating greater efficacy of the IP Group over the Control Group;
Changes from baseline in visual acuity were similar between IP Group and Control Group;
All patients scored 0 for AC cell and AC flare at Day 30;
There was no post-operative complication in either group; and
There was no significant post-operative discomfort in either group at all time periods.

Conclusions of this Study

This multicenter, masked, prospective study demonstrated compounding combination medication (the IP Group) was non-inferior to conventional brand name medication (the Control Group). In fact, the IP Group was shown to be superior to the Control Group, e.g., at Day 30 and with respect to metrics of corneal staining and IOP.

Patient compliance may be improved with this combination therapy (the IP Group) and has been shown to be safe and therapeutically equivalent or superior to conventional multiple medication therapy (Control Group) for postoperative management following LASIK.

Amniotic Cytokines Extract (ACE) Study

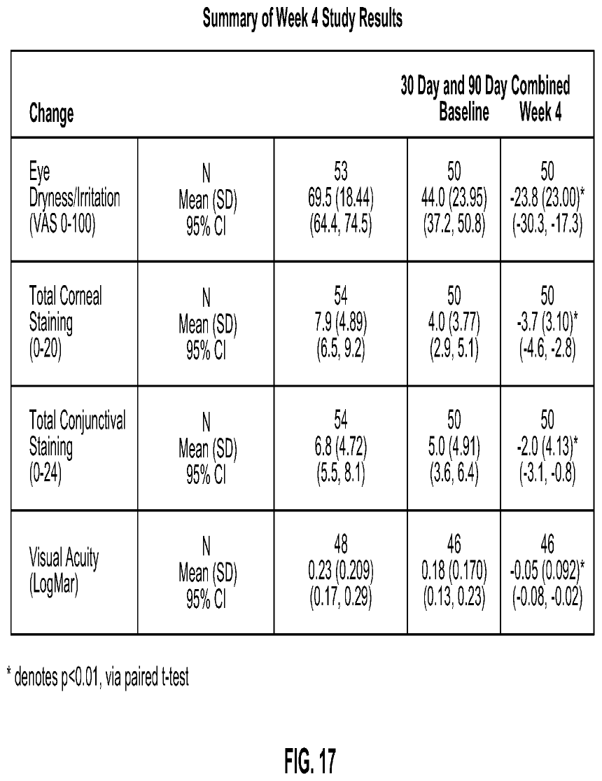

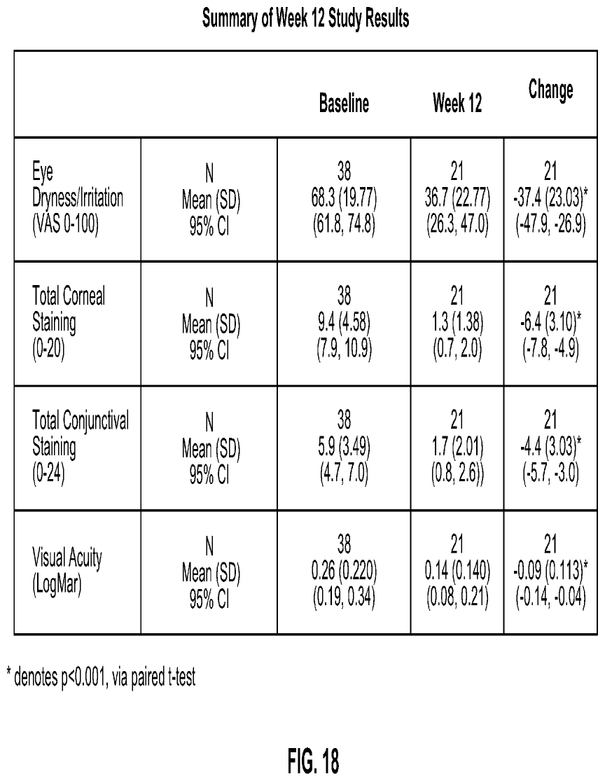

FIG. 17 through FIG. 23 show study results of a pharmaceutical composition comprising amniotic cytokine extract in an eye dropper delivery device for the treatment of dry eyes. A purpose of this study may have been to evaluate an amniotic cytokine extract (ACE) pharmaceutical composition in the treatment of dry eye disease.

Dry eye disease (DES) may be a complex and multifactorial condition—making it difficult to test with existing molecules. Dry eye disease may involve multiple inflammatory pathways. There may be a disconnect between when signs and symptoms can occur, which may further complicate diagnosis. There is a need for novel therapies for treating dry eye disease.

The pharmaceutical composition used in this study comprised an extract of amniotic cytokines and was administered via eye drops. This extract of amniotic cytokines may have comprised active cytokines, growth factors, and anti-inflammatory molecules. This extract of amniotic cytokines may have comprised over 120 active cytokines. This extract of amniotic cytokines may have comprised PGE2. This extract of amniotic cytokines may have comprised GDF11. This extract of amniotic cytokines may have comprised WNT4. This extract of amniotic cytokines may have comprised Thrombospondin-1. PGE2 may promote wound healing. GDF11 may promote inflammation modulation. WNT4 may promote regeneration. Thrombospondin-1 may modulate WBC, as well as dendritic cells/APC that stimulate T cell proliferation. This pharmaceutical composition may have been titrated to a specific concentration of PGE2.

A proprietary cryopreservation technique may have been used to harvest this cytokine extract from amniotic tissue in a manner that preserves important anti-inflammatory molecules for treating ocular surface inflammation that mediates dry eye disease.

Methods of this Study

This study included a retrospective chart review including 53 patients from 7 US clinicians who treated symptomatic dry eye disease patients with the pharmaceutical composition comprising the amniotic cytokine extract (ACE) described above, administered as eye drops.

All patients in the study used this pharmaceutical composition of ACE as one drop twice daily for 12 weeks.

The study eye was defined as the eye with the highest total corneal staining score (TCSS) at baseline.

Following parameters were assessed at baseline, 4 weeks, 8 weeks and 12 weeks after treatment:
Visual acuity;
Symptom score assessed as Eye Dryness Score (EDS) based on a visual analog scale (0-100);
Conjunctival and corneal staining scores using lissamine green and sodium fluorescein; and
Adverse events.

Results of this Study

Results of this study may be depicted in figures FIG. 17 through FIG. 23.

FIG. 17 may depict a table from a study on a pharmaceutical composition comprising PGE2 and active cytokines from amniotic cytokine extract; wherein FIG. 17 may depict summary of week 4 study results.

FIG. 18 may depict a table from a study on a pharmaceutical composition comprising PGE2 and active cytokines from amniotic cytokine extract; wherein FIG. 18 may depict summary of week 12 study results.

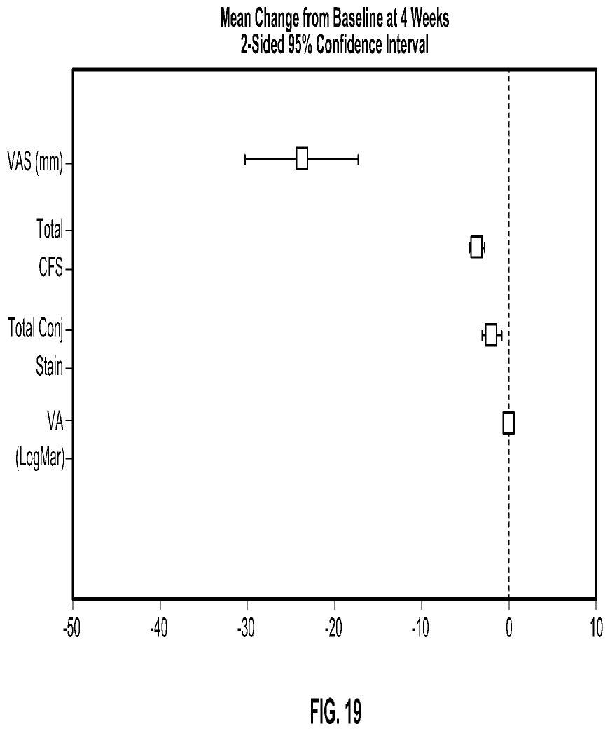

FIG. 19 may depict a table from a study on a pharmaceutical composition comprising PGE2 and active cytokines from amniotic cytokine extract; wherein FIG. 19 may depict mean change from baseline at 4 weeks, 2-sided 95% confident interval (CI).

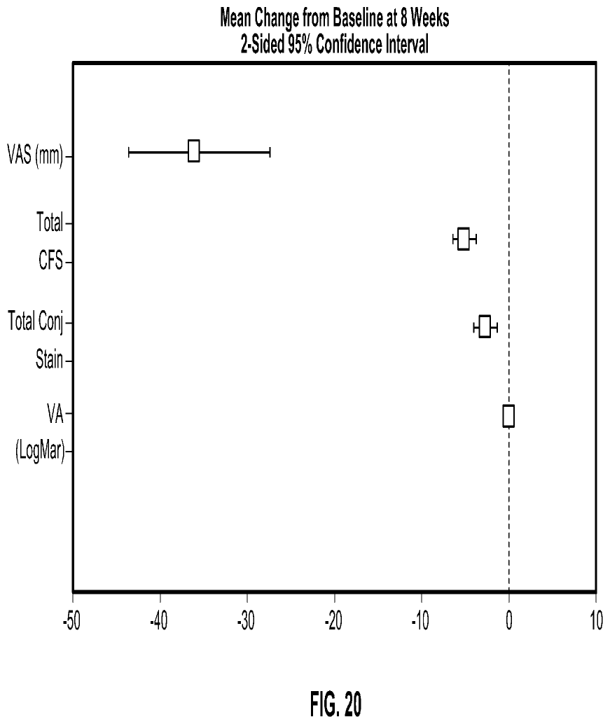

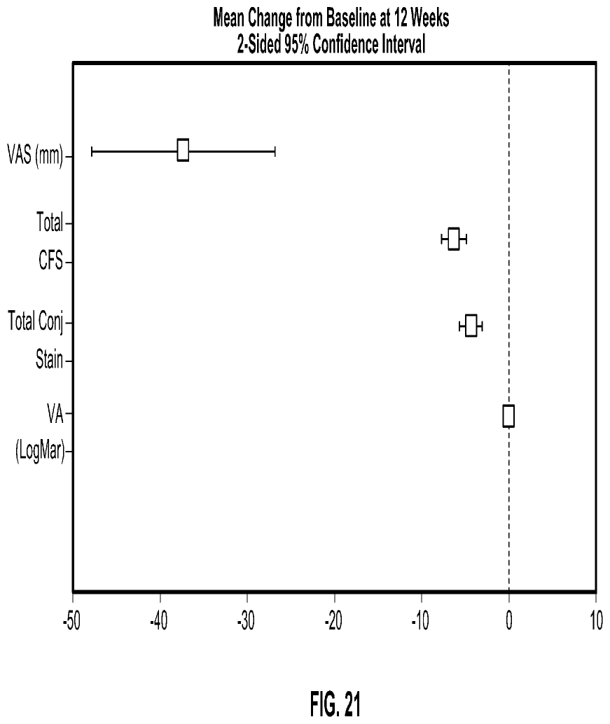

FIG. 20 may depict a table from a study on a pharmaceutical composition comprising PGE2 and active cytokines from amniotic cytokine extract; wherein FIG. 20 may depict mean change from baseline at 8 weeks, 2-sided 95% confident interval (CI).

FIG. 21 may depict a table from a study on a pharmaceutical composition comprising PGE2 and active cytokines from amniotic cytokine extract; wherein FIG. 21 may depict mean change from baseline at 12 weeks, 2-sided 95% confident interval (CI).

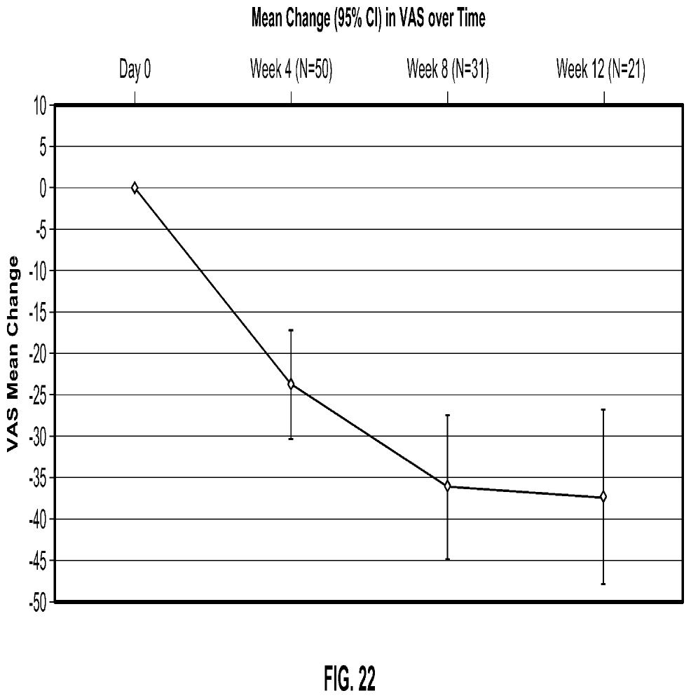

FIG. 22 may depict a table from a study on a pharmaceutical composition comprising PGE2 and active cytokines from amniotic cytokine extract; wherein FIG. 22 may depict mean change (95% CI) in VAS over time.

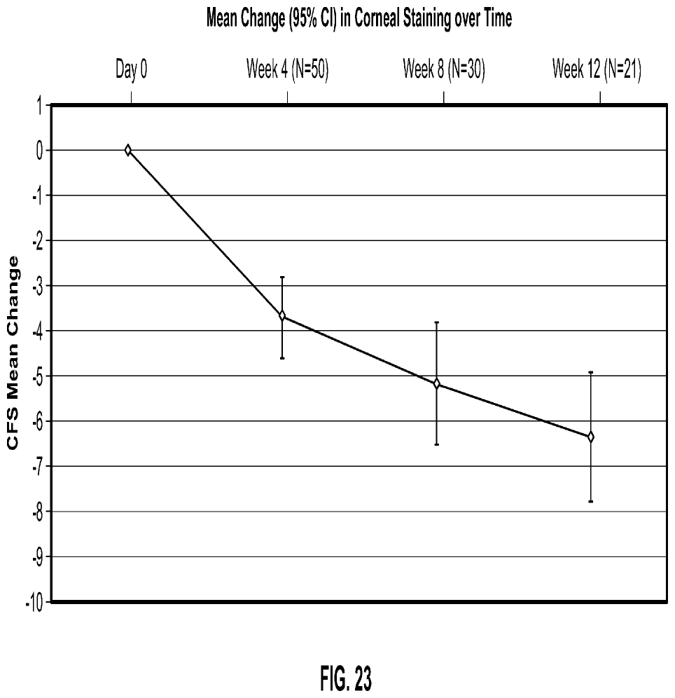

FIG. 23 may depict a table from a study on a pharmaceutical composition comprising PGE2 and active cytokines from amniotic cytokine extract; wherein FIG. 23 may depict mean change (95% CI) in corneal staining over time.

Some of these Results May be Summarized as Follows:
Visual acuity remained unchanged in all patients;
There were no adverse events reported;
Primary endpoints:
Mean Eye Dryness Score improved over 12 weeks from 68.3 to 36.7 (p<0.001); 94% of subjects showed an improvement on VAS score from baseline;
Mean Total Corneal Staining Score improved over 12 weeks from 9.4 to 1.3 (p<0.001);
95% of subjects sowed an improvement of at least 50% on TCS from baseline;
Secondary endpoints:
Mean Total Conjunctival Staining Score improved from 5.9 to 1.7 (p<0.001); 95% of subjects showed an improvement in total conjunctival staining from baseline.
An unexpected result of eyelash growth was also seen.

Conclusions of this Study

Pharmaceutical compositions comprising amniotic cytokines extract (ACE) as described, is a safe, effective, and novel therapy for the treatment of dry eye disease.

Key preservation techniques allow retention of beneficial cytokine activity.

This pharmaceutical composition comprising amniotic cytokines extract (ACE) may also promote and/or facilitate eyelash growth.

In some embodiments, any of the above discussed pharmaceutical compositions may be ophthalmic solutions, used to treat the eye, at the eye, in the eye, and/or around the eye.

Note that the various acids and bases used in these pharmaceutical compositions (e.g., to adjust pH and/or tonicity during compounding) may not exist as acids nor bases once used in the given pharmaceutical composition, as such acids and bases may dissociate in solution into water and their ionic forms (e.g., Na+, Cl−, etc.).

In preparing the above discussed pharmaceutical compositions, in weighing out dry (e.g., power form) of a given API for a given target concentration (e.g., by weight by volume, w/v), it may be necessary to use the source API's certificate of analysis (CofA) and to use the molecular weight of that given API to calculate a multiplication factor or correction factor. For example, use of a correction factor (multiplication factor) may be needed because: (1) the given API is hygroscopic, taking some degree of water, wherein the CofA should report that amount of water and should be accounted for; and/or (2) assessment of a salt, acid, and/or a base form of the given API may be required depending upon a standard of reference for the given API (e.g., as specified by the USP), molecular weight of a salt form will be different from that of its corresponding free base or free acid form, and calculation of such different molecular weight ratios may determine this correction factor (multiplication factor). For example, pharmaceutical compositions including prednisolone PO₄ may require such a correction; whereas, pharmaceutical compositions including ketorolac tromethamine may not require such a correction. For example, pharmaceutical compositions including timolol and/or brimonidine tartrate may require such a correction. For example, pharmaceutical compositions including moxifloxacin and/or dexamethasone phosphate may require such a correction. For example, pharmaceutical compositions including gatifloxacin may require such a correction. For example, pharmaceutical compositions including bromfenac may require such a correction.

Compositions (e.g., pharmaceutical compositions), methods for treating various issues of the eyes, and methods of preparing such compositions have been described. The foregoing description of the various exemplary embodiments of the invention has been presented for the purposes of illustration and disclosure. It is not intended to be exhaustive or to limit the invention to the precise form disclosed. Many modifications and variations are possible in light of the above teaching without departing from the spirit of the invention.

In another aspect, recognized herein is the need for the development of preservative-free ocular formulations that contain multiple APIs in a single dropper bottle for the purposes of providing post-operative ocular care.

Provided herein, in one aspect, is a method for treating an ocular condition of an eye, comprising administering a pharmaceutical composition at, in, or around the eye via a delivery device and per a predetermined dosing regimen, wherein:

the pharmaceutical composition is free of preservatives;

the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%;
   wherein these percentages are with respect to weight per volume; and
the ocular condition is care after cataract surgery, care after LASIK surgery, care for a retina of the eye after cataract surgery, care for a retina of the eye after retina surgery, in preparation for an intraocular procedure, or during the intraocular procedure.

Pharmaceutical Composition 1: Prednisolone PO₄ 1%/Moxifloxacin HCl 0.5%/Bromfenac 0.075%

In some embodiments, the pharmaceutical composition comprises prednisolone PO₄ about 1%, moxifloxacin HCl about 0.5%, and bromfenac about 0.075%.

In some embodiments, the pharmaceutical composition comprises prednisolone or a pharmaceutically acceptable salt or analog thereof. In some embodiments, the pharmaceutical composition comprises prednisolone. In some embodiments, the pharmaceutical composition comprises prednisolone sodium phosphate (prednisolone PO₄). In some embodiments, the pharmaceutical composition comprises prednisolone acetate. In some embodiments, the pharmaceutical composition comprises prednisolone hemisuccinate sodium salt. In some embodiments, the pharmaceutical composition comprises prednisolone hemisuccinate. In some embodiments, the pharmaceutical composition comprises prednisolone hexanoate. In some embodiments, the pharmaceutical composition comprises prednisolone pivalate. In some embodiments, the pharmaceutical composition comprises prednisolone tebutate.

In some embodiments, prednisolone PO₄ may be known as prednisolone, prednisolone phosphate, prednisolone NaPO₄, or prednisolone sodium phosphate. In some embodiments, prednisolone PO₄ may be prepared to meet USP monograph for prednisolone sodium phosphate ophthalmic solution. In some embodiments, prednisolone PO₄ may be an anti-inflammatory steroid. A mechanism of action for prednisolone PO₄ may be inhibition of migration of polymorphonuclear leukocytes and capilla increase reversal. In some embodiments, prednisolone PO₄ may be used for treating inflammation in the eye, at the eye, and/or around the eye via use of eye drops. In some embodiments, prednisolone PO₄ may be used in preparation for ocular surgery. In some embodiments, prednisolone PO₄ may be used during ocular surgery. In some embodiments, prednisolone PO₄ may be used after ocular surgery.

In some embodiments, the pharmaceutical composition comprises prednisolone in an amount of about 1% with respect to weight per volume. In some embodiments, the pharmaceutical composition comprises from 0.9% to 1.1% prednisolone. In some embodiments, the pharmaceutical composition comprises from about 0.9% to about 1.0% prednisolone. In some embodiments, the pharmaceutical composition comprises from about 1.0% to about 1.1% prednisolone. In some embodiments, the pharmaceutical composition comprises from about 0.90% to about 0.95% prednisolone. In some embodiments, the pharmaceutical composition comprises from about 0.95% to about 1.00% prednisolone. In some embodiments, the pharmaceutical composition comprises from about 1.00% to about 1.05% prednisolone. In some embodiments, the pharmaceutical composition comprises from about 1.05% to about 1.10% prednisolone. In some embodiments, the pharmaceutical composition comprises 0.90% prednisolone. In some embodiments, the pharmaceutical composition comprises 0.91% prednisolone. In some embodiments, the pharmaceutical composition comprises 0.92% prednisolone. In some embodiments, the pharmaceutical composition comprises 0.93% prednisolone. In some embodiments, the pharmaceutical composition comprises 0.94% prednisolone. In some embodiments, the pharmaceutical composition comprises 0.95% prednisolone. In some embodiments, the pharmaceutical composition comprises 0.96% prednisolone. In some embodiments, the pharmaceutical composition comprises 0.97% prednisolone. In some embodiments, the pharmaceutical composition comprises 0.98% prednisolone. In some embodiments, the pharmaceutical composition comprises 0.99% prednisolone. In some embodiments, the pharmaceutical composition comprises 1.00% prednisolone. In some embodiments, the pharmaceutical composition comprises 1.01% prednisolone. In some embodiments, the pharmaceutical composition comprises 1.02% prednisolone. In some embodiments, the pharmaceutical composition comprises 1.03% prednisolone. In some embodiments, the pharmaceutical composition comprises 1.04% prednisolone. In some embodiments, the pharmaceutical composition comprises 1.05% prednisolone. In some embodiments, the pharmaceutical composition comprises 1.06% prednisolone. In some embodiments, the pharmaceutical composition comprises 1.07% prednisolone. In some embodiments, the pharmaceutical composition comprises 1.08% prednisolone. In some embodiments, the pharmaceutical composition comprises 1.09% prednisolone. In some embodiments, the pharmaceutical composition comprises 1.10% prednisolone.

In some embodiments, the pharmaceutical composition comprises moxifloxacin or a pharmaceutically acceptable salt thereof. In some embodiments, moxifloxacin HCl may be known as moxifloxacin or moxifloxacin hydrochloride. In some embodiments, moxifloxacin HCl may be a synthetic fluoroquinolone antibacterial agent. In some embodiments, moxifloxacin may be used in an ophthalmic solution. In some embodiments, moxifloxacin may be used for the treatment of bacterial conjunctivitis (i.e., pink eye). Mechanism of action of moxifloxacin HCl may be through inhibition of DNA gyrase and topoisomerase IV which may be required for some bacterial DNA replication, transcription, repair, and/or recombination.

In some embodiments, the pharmaceutical composition comprises moxifloxacin in an amount of about 0.5% with respect to weight per volume. In some embodiments, the pharmaceutical composition comprises from 0.45% to 0.55% moxifloxacin. In some embodiments, the pharmaceutical composition comprises from 0.45% to 0.50% moxifloxacin. In some embodiments, the pharmaceutical composition comprises from 0.50% to 0.55% moxifloxacin. In some embodiments, the pharmaceutical composition comprises 0.45% moxifloxacin. In some embodiments, the pharmaceutical composition comprises 0.46% moxifloxacin. In some embodiments, the pharmaceutical composition comprises 0.47% moxifloxacin. In some embodiments, the pharmaceutical composition comprises 0.48% moxifloxacin. In some embodiments, the pharmaceutical composition comprises 0.49% moxifloxacin. In some embodiments, the pharmaceutical composition comprises 0.50% moxifloxacin. In some embodiments, the pharmaceutical composition comprises 0.51% moxifloxacin. In some embodiments, the pharmaceutical composition comprises 0.52% moxifloxacin. In some embodiments, the pharmaceutical composition comprises 0.53% moxifloxacin. In some embodiments, the pharmaceutical composition comprises 0.54% moxifloxacin. In some embodiments, the pharmaceutical composition comprises 0.55% moxifloxacin.

In some embodiments, the pharmaceutical composition comprises bromfenac or a pharmaceutically acceptable salt thereof. In some embodiments, bromfenac may be known as bromfenac, bromfenac sodium, and bromfenac ophthalmic solution. In some embodiments, the bromfenac may be known as bromfenac sodium sesquihydrate. In some embodiments, the bromfenac may be a non-steroidal anti-inflammatory drug (NSAID). In some embodiments, bromfenac may block prostaglandin synthesis through cyclooxygenase inhibition, demonstrating COX-2 preference with a lesser affinity for COX-1. In some embodiments, bromfenac may be used as an analgesic. In some embodiments, bromfenac may be used to treat ocular pain. In some embodiments, bromfenac may be used to treat ocular inflammation. In some embodiments, bromfenac may be used to treat, promote, and/or facilitate post eye surgery healing and/or health.

In some embodiments, the pharmaceutical composition comprises bromfenac in an amount of about 0.075% with respect to weight per volume. In some embodiments, the pharmaceutical composition comprises from 0.0675% to 0.0825% moxifloxacin. In some embodiments, the pharmaceutical composition comprises from 0.0675% to 0.0700% moxifloxacin. In some embodiments, the pharmaceutical composition comprises from 0.0700% to 0.0725% moxifloxacin. In some embodiments, the pharmaceutical composition comprises from 0.0725% to 0.0750% moxifloxacin. In some embodiments, the pharmaceutical composition comprises from 0.0750% to 0.0775% moxifloxacin. In some embodiments, the pharmaceutical composition comprises from 0.0775% to 0.0800% moxifloxacin. In some embodiments, the pharmaceutical composition comprises from 0.0800% to 0.0825% moxifloxacin. In some embodiments, the pharmaceutical composition comprises 0.0675% bromfenac. In some embodiments, the pharmaceutical composition comprises 0.0680% bromfenac. In some embodiments, the pharmaceutical composition comprises 0.0685% bromfenac. In some embodiments, the pharmaceutical composition comprises 0.0690% bromfenac. In some embodiments, the pharmaceutical composition comprises 0.0695% bromfenac. In some embodiments, the pharmaceutical composition comprises 0.0700% bromfenac. In some embodiments, the pharmaceutical composition comprises 0.0705% bromfenac. In some embodiments, the pharmaceutical composition comprises 0.0710% bromfenac. In some embodiments, the pharmaceutical composition comprises 0.0715% bromfenac. In some embodiments, the pharmaceutical composition comprises 0.0720% bromfenac. In some embodiments, the pharmaceutical composition comprises 0.0725% bromfenac. In some embodiments, the pharmaceutical composition comprises 0.0730% bromfenac. In some embodiments, the pharmaceutical composition comprises 0.0735% bromfenac. In some embodiments, the pharmaceutical composition comprises 0.0740% bromfenac. In some embodiments, the pharmaceutical composition comprises 0.0745% bromfenac. In some embodiments, the pharmaceutical composition comprises 0.0750% bromfenac. In some embodiments, the pharmaceutical composition comprises 0.0755% bromfenac. In some embodiments, the pharmaceutical composition comprises 0.0760% bromfenac. In some embodiments, the pharmaceutical composition comprises 0.0765% bromfenac. In some embodiments, the pharmaceutical composition comprises 0.0770% bromfenac. In some embodiments, the pharmaceutical composition comprises 0.0775% bromfenac. In some embodiments, the pharmaceutical composition comprises 0.0780% bromfenac. In some embodiments, the pharmaceutical composition comprises 0.0785% bromfenac. In some embodiments, the pharmaceutical composition comprises 0.0790% bromfenac. In some embodiments, the pharmaceutical composition comprises 0.0795% bromfenac. In some embodiments, the pharmaceutical composition comprises 0.0800% bromfenac. In some embodiments, the pharmaceutical composition comprises 0.0805% bromfenac. In some embodiments, the pharmaceutical composition comprises 0.0810% bromfenac. In some embodiments, the pharmaceutical composition comprises 0.0815% bromfenac. In some embodiments, the pharmaceutical composition comprises 0.0820% bromfenac. In some embodiments, the pharmaceutical composition comprises 0.0825% bromfenac.

In some embodiments, the pH of the pharmaceutical composition is about 8. In some embodiments, the pH of the pharmaceutical composition is greater than 8. In some embodiments, the pH of the pharmaceutical composition is 7.0. In some embodiments, the pH of the pharmaceutical composition is 7.1. In some embodiments, the pH of the pharmaceutical composition is 7.2. In some embodiments, the pH of the pharmaceutical composition is 7.3. In some embodiments, the pH of the pharmaceutical composition is 7.4. In some embodiments, the pH of the pharmaceutical composition is 7.5. In some embodiments, the pH of the pharmaceutical composition is 7.6. In some embodiments, the pH of the pharmaceutical composition is 7.7. In some embodiments, the pH of the pharmaceutical composition is 7.8. In some embodiments, the pH of the pharmaceutical composition is 7.9. In some embodiments, the pH of the pharmaceutical composition is 8.0. In some embodiments, the pH of the pharmaceutical composition is 8.1. In some embodiments, the pH of the pharmaceutical composition is 8.2. In some embodiments, the pH of the pharmaceutical composition is 8.3. In some embodiments, the pH of the pharmaceutical composition is 8.4. In some embodiments, the pH of the pharmaceutical composition is 8.5. In some embodiments, the pH of the pharmaceutical composition is 8.6. In some embodiments, the pH of the pharmaceutical composition is 8.7. In some embodiments, the pH of the pharmaceutical composition is 8.8. In some embodiments, the pH of the pharmaceutical composition is 8.9. In some embodiments, the pH of the pharmaceutical composition is 9.0.

In some embodiments, compounding the pharmaceutical composition comprising 1% Prednisolone PO₄, 0.5% Moxifloxacin HCl, and 0.075% Bromfenac may comprise steps of: (step 101) prepping clean work area (e.g., cleaning and/or disinfecting); (step 102) using only sterilized and/or depyrogenated equipment; (step 103) weighing applicable APIs (e.g., Prednisolone PO₄, Moxifloxacin HCl, and Bromfenac) in a powder hood (with the 1%, 0.5%, and 0.075% targets in mind); (step 104) dissolving weighed out API powders in sterile water (or SWFI) (with the 1%, 0.5%, and 0.075% targets in mind); (step 105) testing and adjusting the pH to a target of >8 via use of sodium hydroxide and pH meter (calibrated); (step 106) qs (“quantity sufficient”) with the sterile water (or SWFI) with the 1%, 0.5%, and 0.075% targets in mind; (step 107) transferring resulting solution to a compounding aseptic isolator (CAI); (step 108) sterile filtering (e.g., a 0.22 micron filter) the resulting solution to yield the pharmaceutical composition comprising 1% Prednisolone PO₄, 0.5% Moxifloxacin HCl, and 0.075% Bromfenac; (step 109) QA/QC (quality assurance/quality control) tests, such as bubble point testing, sterility testing, and/or endotoxin testing; (step 110) and filling final delivery device, e.g., a sterile ophthalmic dropper bottle (e.g., a “drop-tainer,” “steri-dropper,” or the like); and (step 111) of label and storage. See e.g., FIG. 1. In some embodiments, the final delivery device, e.g., the sterile ophthalmic dropper bottle, may be light resistant.

### Pharmaceutical Composition 2: Prednisolone PO₄ 1%/Moxifloxacin HCl 0.5%

In some embodiments, the pharmaceutical composition comprises prednisolone PO₄ about 1% and moxifloxacin HCl about 0.5%.

In some embodiments, the pharmaceutical composition comprises prednisolone or a pharmaceutically acceptable salt or analog thereof. In some embodiments, the pharmaceutical composition comprises prednisolone. In some embodiments, the pharmaceutical composition comprises prednisolone sodium phosphate (prednisolone PO₄). In some embodiments, the pharmaceutical composition comprises prednisolone acetate. In some embodiments, the pharmaceutical composition comprises prednisolone hemisuccinate sodium salt. In some embodiments, the pharmaceutical composition comprises prednisolone hemisuccinate. In some embodiments, the pharmaceutical composition comprises prednisolone hexanoate. In some embodiments, the pharmaceutical composition comprises prednisolone pivalate. In some embodiments, the pharmaceutical composition comprises prednisolone tebutate.

In some embodiments, prednisolone PO₄ may be known as prednisolone, prednisolone phosphate, prednisolone NaPO₄, or prednisolone sodium phosphate. In some embodiments, prednisolone PO₄ may be prepared to meet USP monograph for prednisolone sodium phosphate ophthalmic solution. In some embodiments, prednisolone PO₄ may be an anti-inflammatory steroid. A mechanism of action for prednisolone PO₄ may be inhibition of migration of polymorphonuclear leukocytes and capilla increase reversal. In some embodiments, prednisolone PO₄ may be used for treating inflammation in the eye, at the eye, and/or around the eye via use of eye drops. In some embodiments, prednisolone PO₄ may be used in preparation for ocular surgery. In some embodiments, prednisolone PO₄ may be used during ocular surgery. In some embodiments, prednisolone PO₄ may be used after ocular surgery.

In some embodiments, the pharmaceutical composition comprises prednisolone in an amount of about 1% with respect to weight per volume. In some embodiments, the pharmaceutical composition comprises from 0.9% to 1.1% prednisolone. In some embodiments, the pharmaceutical composition comprises from about 0.9% to about 1.0% prednisolone. In some embodiments, the pharmaceutical composition comprises from about 1.0% to about 1.1% prednisolone. In some embodiments, the pharmaceutical composition comprises from about 0.90% to about 0.95% prednisolone. In some embodiments, the pharmaceutical composition comprises from about 0.95% to about 1.00% prednisolone. In some embodiments, the pharmaceutical composition comprises from about 1.00% to about 1.05% prednisolone. In some embodiments, the pharmaceutical composition comprises from about 1.05% to about 1.10% prednisolone. In some embodiments, the pharmaceutical composition comprises 0.90% prednisolone. In some embodiments, the pharmaceutical composition comprises 0.91% prednisolone. In some embodiments, the pharmaceutical composition comprises 0.92% prednisolone. In some embodiments, the pharmaceutical composition comprises 0.93% prednisolone. In some embodiments, the pharmaceutical composition comprises 0.94% prednisolone. In some embodiments, the pharmaceutical composition comprises 0.95% prednisolone. In some embodiments, the pharmaceutical composition comprises 0.96% prednisolone. In some embodiments, the pharmaceutical composition comprises 0.97% prednisolone. In some embodiments, the pharmaceutical composition comprises 0.98% prednisolone. In some embodiments, the pharmaceutical composition comprises 0.99% prednisolone. In some embodiments, the pharmaceutical composition comprises 1.00% prednisolone. In some embodiments, the pharmaceutical composition comprises 1.01% prednisolone. In some embodiments, the pharmaceutical composition comprises 1.02% prednisolone. In some embodiments, the pharmaceutical composition comprises 1.03% prednisolone. In some embodiments, the pharmaceutical composition comprises 1.04% prednisolone. In some embodiments, the pharmaceutical composition comprises 1.05% prednisolone. In some embodiments, the pharmaceutical composition comprises 1.06% prednisolone. In some embodiments, the pharmaceutical composition comprises 1.07% prednisolone. In some embodiments, the pharmaceutical composition comprises 1.08% prednisolone. In some embodiments, the pharmaceutical composition comprises 1.09% prednisolone. In some embodiments, the pharmaceutical composition comprises 1.10% prednisolone.

In some embodiments, the pharmaceutical composition comprises moxifloxacin or a pharmaceutically acceptable salt thereof. In some embodiments, moxifloxacin HCl may be known as moxifloxacin or moxifloxacin hydrochloride. In some embodiments, moxifloxacin HCl may be a synthetic fluoroquinolone antibacterial agent. In some embodiments, moxifloxacin may be used in an ophthalmic solution. In some embodiments, moxifloxacin may be used for the treatment of bacterial conjunctivitis (i.e., pink eye). Mechanism of action of moxifloxacin HCl may be through inhibition of DNA gyrase and topoisomerase IV which may be required for some bacterial DNA replication, transcription, repair, and/or recombination.

In some embodiments, the pharmaceutical composition comprises moxifloxacin in an amount of about 0.5% with respect to weight per volume. In some embodiments, the pharmaceutical composition comprises from 0.45% to 0.55% moxifloxacin. In some embodiments, the pharmaceutical composition comprises from 0.45% to 0.50% moxifloxacin. In some embodiments, the pharmaceutical composition comprises from 0.50% to 0.55% moxifloxacin. In some embodiments, the pharmaceutical composition comprises 0.45% moxifloxacin. In some embodiments, the pharmaceutical composition comprises 0.46% moxifloxacin. In some embodiments, the pharmaceutical composition comprises 0.47% moxifloxacin. In some embodiments, the pharmaceutical composition comprises 0.48% moxifloxacin. In some embodiments, the pharmaceutical composition comprises 0.49% moxifloxacin. In some embodiments, the pharmaceutical composition comprises 0.50% moxifloxacin. In some embodiments, the pharmaceutical composition comprises 0.51% moxifloxacin. In some embodiments, the pharmaceutical composition comprises 0.52% moxifloxacin. In some embodiments, the pharmaceutical composition comprises 0.53% moxifloxacin. In some embodiments, the pharmaceutical composition comprises 0.54% moxifloxacin. In some embodiments, the pharmaceutical composition comprises 0.55% moxifloxacin.

In some embodiments, the pH of the pharmaceutical composition is about 7. In some embodiments, the pH of the pharmaceutical composition is greater than 7. In some embodiments, the pH of the pharmaceutical composition is 7.0. In some embodiments, the pH of the pharmaceutical composition is 7.1. In some embodiments, the pH of the pharmaceutical composition is 7.2. In some embodiments, the pH of the pharmaceutical composition is 7.3. In some embodiments, the pH of the pharmaceutical composition is 7.4. In some embodiments, the pH of the pharmaceutical composition is 7.5. In some embodiments, the pH of the pharmaceutical composition is 7.6. In some embodiments, the pH of the pharmaceutical composition is 7.7. In some embodiments, the pH of the pharmaceutical composition is 7.8. In some embodiments, the pH of the pharmaceutical composition is 7.9. In some embodiments, the pH of the pharmaceutical composition is 8.0.

In some embodiments, compounding the pharmaceutical composition comprising 1% Prednisolone PO₄ and 0.5% Moxifloxacin HCl may comprise steps of: (step 101) prepping clean work area (e.g., cleaning and/or disinfecting); (step 102) using only sterilized and/or depyrogenated equipment; (step 103) weighing applicable APIs (e.g., Prednisolone PO₄ and Moxifloxacin HCl) in a powder hood (with the 1% and 0.5% targets in mind); (step 104) dissolving weighed out API powders in sterile water (or SWFI) (with the 1% and 0.5% targets in mind); (step 105) testing and adjusting the pH to a target of >7 via use of sodium hydroxide and pH meter (calibrated); (step 106) qs (“quantity sufficient”) with the sterile water (or SWFI) with the 1% and 0.5% targets in mind; (step 107) transferring resulting solution to a compounding aseptic isolator (CAI); (step 108) sterile filtering (e.g., a 0.22 micron filter) the resulting solution to yield the pharmaceutical composition comprising 1% Prednisolone PO₄ and 0.5% Moxifloxacin HCl; (step 109) QA/QC (quality assurance/quality control) tests, such as bubble point testing, sterility testing, and/or endotoxin testing; (step 110) and filling final delivery device, e.g., a sterile ophthalmic dropper bottle (e.g., a “drop-tainer,” “steri-dropper,” or the like); and (step 111) of label and storage. See e.g., FIG. 1. In some embodiments, the final delivery device, e.g., the sterile ophthalmic dropper bottle, may be light resistant.

### Pharmaceutical Composition 3: Moxifloxacin HCl 0.5%/Bromfenac 0.075%

In some embodiments, the pharmaceutical composition comprises moxifloxacin HCl about 0.5% and bromfenac about 0.075%.

In some embodiments, the pharmaceutical composition comprises moxifloxacin or a pharmaceutically acceptable salt thereof. In some embodiments, moxifloxacin HCl may be known as moxifloxacin or moxifloxacin hydrochloride. In some embodiments, moxifloxacin HCl may be a synthetic fluoroquinolone antibacterial agent. In some embodiments, moxifloxacin may be used in an ophthalmic solution. In some embodiments, moxifloxacin may be used for the treatment of bacterial conjunctivitis (i.e., pink eye). Mechanism of action of moxifloxacin HCl may be through inhibition of DNA gyrase and topoisomerase IV which may be required for some bacterial DNA replication, transcription, repair, and/or recombination.

In some embodiments, the pharmaceutical composition comprises moxifloxacin in an amount of about 0.5% with respect to weight per volume. In some embodiments, the pharmaceutical composition comprises from 0.45% to 0.55% moxifloxacin. In some embodiments, the pharmaceutical composition comprises from 0.45% to 0.50% moxifloxacin. In some embodiments, the pharmaceutical composition comprises from 0.50% to 0.55% moxifloxacin. In some embodiments, the pharmaceutical composition comprises 0.45% moxifloxacin. In some embodiments, the pharmaceutical composition comprises 0.46% moxifloxacin. In some embodiments, the pharmaceutical composition comprises 0.47% moxifloxacin. In some embodiments, the pharmaceutical composition comprises 0.48% moxifloxacin. In some embodiments, the pharmaceutical composition comprises 0.49% moxifloxacin. In some embodiments, the pharmaceutical composition comprises 0.50% moxifloxacin. In some embodiments, the pharmaceutical composition comprises 0.51% moxifloxacin. In some embodiments, the pharmaceutical composition comprises 0.52% moxifloxacin. In some embodiments, the pharmaceutical composition comprises 0.53% moxifloxacin. In some embodiments, the pharmaceutical composition comprises 0.54% moxifloxacin. In some embodiments, the pharmaceutical composition comprises 0.55% moxifloxacin.

In some embodiments, the pharmaceutical composition comprises bromfenac or a pharmaceutically acceptable salt thereof. In some embodiments, bromfenac may be known as bromfenac, bromfenac sodium, and bromfenac ophthalmic solution. In some embodiments, the bromfenac may be known as bromfenac sodium sesquihydrate. In some embodiments, the bromfenac may be a non-steroidal anti-inflammatory drug (NSAID). In some embodiments, bromfenac may block prostaglandin synthesis through cyclooxygenase inhibition, demonstrating COX-2 preference with a lesser affinity for COX-1. In some embodiments, bromfenac may be used as an analgesic. In some embodiments, bromfenac may be used to treat ocular pain. In some embodiments, bromfenac may be used to treat ocular inflammation. In some embodiments, bromfenac may be used to treat, promote, and/or facilitate post eye surgery healing and/or health.

In some embodiments, the pharmaceutical composition comprises bromfenac in an amount of about 0.075% with respect to weight per volume. In some embodiments, the pharmaceutical composition comprises from 0.0675% to 0.0825% moxifloxacin. In some embodiments, the pharmaceutical composition comprises from 0.0675% to 0.0700% moxifloxacin. In some embodiments, the pharmaceutical composition comprises from 0.0700% to 0.0725% moxifloxacin. In some embodiments, the pharmaceutical composition comprises from 0.0725% to 0.0750% moxifloxacin. In some embodiments, the pharmaceutical composition comprises from 0.0750% to 0.0775% moxifloxacin. In some embodiments, the pharmaceutical composition comprises from 0.0775% to 0.0800% moxifloxacin. In some embodiments, the pharmaceutical composition comprises from 0.0800% to 0.0825% moxifloxacin. In some embodiments, the pharmaceutical composition comprises 0.0675% bromfenac. In some embodiments, the pharmaceutical composition comprises 0.0680% bromfenac. In some embodiments, the pharmaceutical composition comprises 0.0685% bromfenac. In some embodiments, the pharmaceutical composition comprises 0.0690% bromfenac. In some embodiments, the pharmaceutical composition comprises 0.0695% bromfenac. In some embodiments, the pharmaceutical composition comprises 0.0700% bromfenac. In some embodiments, the pharmaceutical composition comprises 0.0705% bromfenac. In some embodiments, the pharmaceutical composition comprises 0.0710% bromfenac. In some embodiments, the pharmaceutical composition comprises 0.0715% bromfenac. In some embodiments, the pharmaceutical composition comprises 0.0720% bromfenac. In some embodiments, the pharmaceutical composition comprises 0.0725% bromfenac. In some embodiments, the pharmaceutical composition comprises 0.0730% bromfenac. In some embodiments, the pharmaceutical composition comprises 0.0735% bromfenac. In some embodiments, the pharmaceutical composition comprises 0.0740% bromfenac. In some embodiments, the pharmaceutical composition comprises 0.0745% bromfenac. In some embodiments, the pharmaceutical composition comprises 0.0750% bromfenac. In some embodiments, the pharmaceutical composition comprises 0.0755% bromfenac. In some embodiments, the pharmaceutical composition comprises 0.0760% bromfenac. In some embodiments, the pharmaceutical composition comprises 0.0765% bromfenac. In some embodiments, the pharmaceutical composition comprises 0.0770% bromfenac. In some embodiments, the pharmaceutical composition comprises 0.0775% bromfenac. In some embodiments, the pharmaceutical composition comprises 0.0780% bromfenac. In some embodiments, the pharmaceutical composition comprises 0.0785% bromfenac. In some embodiments, the pharmaceutical composition comprises 0.0790% bromfenac. In some embodiments, the pharmaceutical composition comprises 0.0795% bromfenac. In some embodiments, the pharmaceutical composition comprises 0.0800% bromfenac. In some embodiments, the pharmaceutical composition comprises 0.0805% bromfenac. In some embodiments, the pharmaceutical composition comprises 0.0810% bromfenac. In some embodiments, the pharmaceutical composition comprises 0.0815% bromfenac. In some embodiments, the pharmaceutical composition comprises 0.0820% bromfenac. In some embodiments, the pharmaceutical composition comprises 0.0825% bromfenac.

In some embodiments, the pH of the pharmaceutical composition is about 8. In some embodiments, the pH of the pharmaceutical composition is greater than 8. In some embodiments, the pH of the pharmaceutical composition is 7.0. In some embodiments, the pH of the pharmaceutical composition is 7.1. In some embodiments, the pH of the pharmaceutical composition is 7.2. In some embodiments, the pH of the pharmaceutical composition is 7.3. In some embodiments, the pH of the pharmaceutical composition is 7.4. In some embodiments, the pH of the pharmaceutical composition is 7.5. In some embodiments, the pH of the pharmaceutical composition is 7.6. In some embodiments, the pH of the pharmaceutical composition is 7.7. In some embodiments, the pH of the pharmaceutical composition is 7.8. In some embodiments, the pH of the pharmaceutical composition is 7.9. In some embodiments, the pH of the pharmaceutical composition is 8.0. In some embodiments, the pH of the pharmaceutical composition is 8.1. In some embodiments, the pH of the pharmaceutical composition is 8.2. In some embodiments, the pH of the pharmaceutical composition is 8.3. In some embodiments, the pH of the pharmaceutical composition is 8.4. In some embodiments, the pH of the pharmaceutical composition is 8.5. In some embodiments, the pH of the pharmaceutical composition is 8.6. In some embodiments, the pH of the pharmaceutical composition is 8.7. In some embodiments, the pH of the pharmaceutical composition is 8.8. In some embodiments, the pH of the pharmaceutical composition is 8.9. In some embodiments, the pH of the pharmaceutical composition is 9.0.

In some embodiments, compounding the pharmaceutical composition comprising 0.5% Moxifloxacin HCl and 0.075% Bromfenac may comprise steps of: (step 101) prepping clean work area (e.g., cleaning and/or disinfecting); (step 102) using only sterilized and/or depyrogenated equipment; (step 103) weighing applicable APIs (e.g., Moxifloxacin HCl and Bromfenac) in a powder hood (with the 0.5% and 0.075% targets in mind); (step 104) dissolving weighed out API powders in sterile water (or SWFI) (with the 0.5% and 0.075% targets in mind); (step 105) testing and adjusting the pH to a target of >8 via use of sodium hydroxide and pH meter (calibrated); (step 106) qs (“quantity sufficient”) with the sterile water (or SWFI) with the 0.5% and 0.075% targets in mind; (step 107) transferring resulting solution to a compounding aseptic isolator (CAI); (step 108) sterile filtering (e.g., a 0.22 micron filter) the resulting solution to yield the pharmaceutical composition comprising 0.5% Moxifloxacin HCl and 0.075% Bromfenac; (step 109) QA/QC (quality assurance/quality control) tests, such as bubble point testing, sterility testing, and/or endotoxin testing; (step 110) and filling final delivery device, e.g., a sterile ophthalmic dropper bottle (e.g., a “drop-tainer,” “steri-dropper,” or the like); and (step 111) of label and storage. See e.g., FIG. 1. In some embodiments, the final delivery device, e.g., the sterile ophthalmic dropper bottle, may be light resistant.

### Pharmaceutical Composition 4: Difluprednate 0.05%/Moxifloxacin HCl 0.5%/Bromfenac 0.075%

In some embodiments, the pharmaceutical composition comprises difluprednate about 0.05%, moxifloxacin HCl about 0.5%, and bromfenac about 0.075%.

In some embodiments, the pharmaceutical composition comprises difluprednate or a pharmaceutically acceptable salt thereof. In some embodiments, the pharmaceutical composition comprises difluprednate. In some embodiments, difluprednate may be prepared to meet USP monograph for difluprednate ophthalmic solution. In some embodiments, difluprednate may be an anti-inflammatory steroid. A mechanism of action for difluprednate may be inhibition of migration of polymorphonuclear leukocytes and capilla increase reversal. In some embodiments, difluprednate may be used for treating inflammation in the eye, at the eye, and/or around the eye via use of eye drops. In some embodiments, difluprednate may be used in preparation for ocular surgery. In some embodiments, difluprednate may be used during ocular surgery. In some embodiments, difluprednate may be used after ocular surgery.

In some embodiments, the pharmaceutical composition comprises difluprednate in an amount of about 0.05% with respect to weight per volume. In some embodiments, the pharmaceutical composition comprises from 0.045% to 0.055% difluprednate. In some embodiments, the pharmaceutical composition comprises from about 0.045% to 0.050% difluprednate. In some embodiments, the pharmaceutical composition comprises from about 0.050% to 0.055% difluprednate. In some embodiments, the pharmaceutical composition comprises 0.045% difluprednate. In some embodiments, the pharmaceutical composition comprises 0.046% difluprednate. In some embodiments, the pharmaceutical composition comprises 0.047% difluprednate. In some embodiments, the pharmaceutical composition comprises 0.048% difluprednate. In some embodiments, the pharmaceutical composition comprises 0.049% difluprednate. In some embodiments, the pharmaceutical composition comprises 0.050% difluprednate. In some embodiments, the pharmaceutical composition comprises 0.051% difluprednate. In some embodiments, the pharmaceutical composition comprises 0.052% difluprednate. In some embodiments, the pharmaceutical composition comprises 0.053% difluprednate. In some embodiments, the pharmaceutical composition comprises 0.054% difluprednate. In some embodiments, the pharmaceutical composition comprises 0.055% difluprednate.

In some embodiments, the pharmaceutical composition comprises moxifloxacin or a pharmaceutically acceptable salt thereof. In some embodiments, moxifloxacin HCl may be known as moxifloxacin or moxifloxacin hydrochloride. In some embodiments, moxifloxacin HCl may be a synthetic fluoroquinolone antibacterial agent. In some embodiments, moxifloxacin may be used in an ophthalmic solution. In some embodiments, moxifloxacin may be used for the treatment of bacterial conjunctivitis (i.e., pink eye). Mechanism of action of moxifloxacin HCl may be through inhibition of DNA gyrase and topoisomerase IV which may be required for some bacterial DNA replication, transcription, repair, and/or recombination.

In some embodiments, the pharmaceutical composition comprises moxifloxacin in an amount of about 0.5% with respect to weight per volume. In some embodiments, the pharmaceutical composition comprises from 0.45% to 0.55% moxifloxacin. In some embodiments, the pharmaceutical composition comprises from 0.45% to 0.50% moxifloxacin. In some embodiments, the pharmaceutical composition comprises from 0.50% to 0.55% moxifloxacin. In some embodiments, the pharmaceutical composition comprises 0.45% moxifloxacin. In some embodiments, the pharmaceutical composition comprises 0.46% moxifloxacin. In some embodiments, the pharmaceutical composition comprises 0.47% moxifloxacin. In some embodiments, the pharmaceutical composition comprises 0.48% moxifloxacin. In some embodiments, the pharmaceutical composition comprises 0.49% moxifloxacin. In some embodiments, the pharmaceutical composition comprises 0.50% moxifloxacin. In some embodiments, the pharmaceutical composition comprises 0.51% moxifloxacin. In some embodiments, the pharmaceutical composition comprises 0.52% moxifloxacin. In some embodiments, the pharmaceutical composition comprises 0.53% moxifloxacin. In some embodiments, the pharmaceutical composition comprises 0.54% moxifloxacin. In some embodiments, the pharmaceutical composition comprises 0.55% moxifloxacin.

In some embodiments, the pharmaceutical composition comprises bromfenac or a pharmaceutically acceptable salt thereof. In some embodiments, bromfenac may be known as bromfenac, bromfenac sodium, and bromfenac ophthalmic solution. In some embodiments, the bromfenac may be known as bromfenac sodium sesquihydrate. In some embodiments, the bromfenac may be a non-steroidal anti-inflammatory drug (NSAID). In some embodiments, bromfenac may block prostaglandin synthesis through cyclooxygenase inhibition, demonstrating COX-2 preference with a lesser affinity for COX-1. In some embodiments, bromfenac may be used as an analgesic. In some embodiments, bromfenac may be used to treat ocular pain. In some embodiments, bromfenac may be used to treat ocular inflammation. In some embodiments, bromfenac may be used to treat, promote, and/or facilitate post eye surgery healing and/or health.

In some embodiments, the pharmaceutical composition comprises bromfenac in an amount of about 0.075% with respect to weight per volume. In some embodiments, the pharmaceutical composition comprises from 0.0675% to 0.0825% moxifloxacin. In some embodiments, the pharmaceutical composition comprises from 0.0675% to 0.0700% moxifloxacin. In some embodiments, the pharmaceutical composition comprises from 0.0700% to 0.0725% moxifloxacin. In some embodiments, the pharmaceutical composition comprises from 0.0725% to 0.0750% moxifloxacin. In some embodiments, the pharmaceutical composition comprises from 0.0750% to 0.0775% moxifloxacin. In some embodiments, the pharmaceutical composition comprises from 0.0775% to 0.0800% moxifloxacin. In some embodiments, the pharmaceutical composition comprises from 0.0800% to 0.0825% moxifloxacin. In some embodiments, the pharmaceutical composition comprises 0.0675% bromfenac. In some embodiments, the pharmaceutical composition comprises 0.0680% bromfenac. In some embodiments, the pharmaceutical composition comprises 0.0685% bromfenac. In some embodiments, the pharmaceutical composition comprises 0.0690% bromfenac. In some embodiments, the pharmaceutical composition comprises 0.0695% bromfenac. In some embodiments, the pharmaceutical composition comprises 0.0700% bromfenac. In some embodiments, the pharmaceutical composition comprises 0.0705% bromfenac. In some embodiments, the pharmaceutical composition comprises 0.0710% bromfenac. In some embodiments, the pharmaceutical composition comprises 0.0715% bromfenac. In some embodiments, the pharmaceutical composition comprises 0.0720% bromfenac. In some embodiments, the pharmaceutical composition comprises 0.0725% bromfenac. In some embodiments, the pharmaceutical composition comprises 0.0730% bromfenac. In some embodiments, the pharmaceutical composition comprises 0.0735% bromfenac. In some embodiments, the pharmaceutical composition comprises 0.0740% bromfenac. In some embodiments, the pharmaceutical composition comprises 0.0745% bromfenac. In some embodiments, the pharmaceutical composition comprises 0.0750% bromfenac. In some embodiments, the pharmaceutical composition comprises 0.0755% bromfenac. In some embodiments, the pharmaceutical composition comprises 0.0760% bromfenac. In some embodiments, the pharmaceutical composition comprises 0.0765% bromfenac. In some embodiments, the pharmaceutical composition comprises 0.0770% bromfenac. In some embodiments, the pharmaceutical composition comprises 0.0775% bromfenac. In some embodiments, the pharmaceutical composition comprises 0.0780% bromfenac. In some embodiments, the pharmaceutical composition comprises 0.0785% bromfenac. In some embodiments, the pharmaceutical composition comprises 0.0790% bromfenac. In some embodiments, the pharmaceutical composition comprises 0.0795% bromfenac. In some embodiments, the pharmaceutical composition comprises 0.0800% bromfenac. In some embodiments, the pharmaceutical composition comprises 0.0805% bromfenac. In some embodiments, the pharmaceutical composition comprises 0.0810% bromfenac. In some embodiments, the pharmaceutical composition comprises 0.0815% bromfenac. In some embodiments, the pharmaceutical composition comprises 0.0820% bromfenac. In some embodiments, the pharmaceutical composition comprises 0.0825% bromfenac.

In some embodiments, the pH of the pharmaceutical composition is about 8. In some embodiments, the pH of the pharmaceutical composition is greater than 8. In some embodiments, the pH of the pharmaceutical composition is 7.0. In some embodiments, the pH of the pharmaceutical composition is 7.1. In some embodiments, the pH of the pharmaceutical composition is 7.2. In some embodiments, the pH of the pharmaceutical composition is 7.3. In some embodiments, the pH of the pharmaceutical composition is 7.4. In some embodiments, the pH of the pharmaceutical composition is 7.5. In some embodiments, the pH of the pharmaceutical composition is 7.6. In some embodiments, the pH of the pharmaceutical composition is 7.7. In some embodiments, the pH of the pharmaceutical composition is 7.8. In some embodiments, the pH of the pharmaceutical composition is 7.9. In some embodiments, the pH of the pharmaceutical composition is 8.0. In some embodiments, the pH of the pharmaceutical composition is 8.1. In some embodiments, the pH of the pharmaceutical composition is 8.2. In some embodiments, the pH of the pharmaceutical composition is 8.3. In some embodiments, the pH of the pharmaceutical composition is 8.4. In some embodiments, the pH of the pharmaceutical composition is 8.5. In some embodiments, the pH of the pharmaceutical composition is 8.6. In some embodiments, the pH of the pharmaceutical composition is 8.7. In some embodiments, the pH of the pharmaceutical composition is 8.8. In some embodiments, the pH of the pharmaceutical composition is 8.9. In some embodiments, the pH of the pharmaceutical composition is 9.0.

In some embodiments, compounding the pharmaceutical composition comprising 0.05% Difluprednate, 0.5% Moxifloxacin HCl, and 0.075% Bromfenac may comprise steps of: (step 101) prepping clean work area (e.g., cleaning and/or disinfecting); (step 102) using only sterilized and/or depyrogenated equipment; (step 103) weighing applicable APIs (e.g., Difluprednate, Moxifloxacin HCl, and Bromfenac) in a powder hood (with the 0.05%, 0.5%, and 0.075% targets in mind); (step 104) dissolving weighed out API powders in sterile water (or SWFI) (with the 0.05%, 0.5%, and 0.075% targets in mind); (step 105) testing and adjusting the pH to a target of >8 via use of sodium hydroxide and pH meter (calibrated); (step 106) qs (“quantity sufficient”) with the sterile water (or SWFI) with the 0.05%, 0.5%, and 0.075% targets in mind; (step 107) transferring resulting solution to ISO 5 area; (step 108) sterile filtering aqueous phase (e.g., a 0.22 micron filter), adding the lipophilic phase, and homogenizing to achieve an emulsion to yield the pharmaceutical composition comprising 0.05% Difluprednate, 0.5% Moxifloxacin HCl, and 0.075% Bromfenac; (step 109) QA/QC (quality assurance/quality control) tests, such as bubble point testing, sterility testing, and/or endotoxin testing; (step 110) and filling final delivery device, e.g., a sterile ophthalmic dropper bottle (e.g., a “drop-tainer,” “steri-dropper,” or the like); and (step 111) of label and storage. See e.g., FIG. 1. In some embodiments, the final delivery device, e.g., the sterile ophthalmic dropper bottle, may be light resistant. In some embodiments the final delivery device with drug may be terminally sterilized.

### Aqueous Solution Stability

In some embodiments, the pharmaceutical composition described herein comprises a buffer. In some embodiments, a buffer is selected from borates, borate-polyol complexes, phosphate buffering agents, citrate buffering agents, acetate buffering agents, carbonate buffering agents, organic buffering agents, amino acid buffering agents, or combinations thereof.

In some embodiments, borates include boric acid, salts of boric acid, other pharmaceutically acceptable borates, and combinations thereof. In some embodiments, borates include boric acid, sodium borate, potassium borate, calcium borate, magnesium borate, manganese borate, and other such borate salts.

As used herein, the term “polyol” includes any compound having at least one hydroxyl group on each of two adjacent carbon atoms that are not in trans configuration relative to each other. In some embodiments, a polyols is linear or cyclic, substituted or unsubstituted, or mixtures thereof, so long as the resultant complex is water soluble and pharmaceutically acceptable. In some embodiments, examples of polyol include: sugars, sugar alcohols, sugar acids, and uronic acids. In some embodiments, polyols include but are not limited to mannitol, glycerin, xylitol, and sorbitol.

In some embodiments, phosphate buffering agents include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; ammonium phosphates such as diammonium hydrogen phosphate and ammonium dihydrogen phosphate; or a combination thereof. In some embodiments, the phosphate buffering agent is an anhydride. In some embodiments, the phosphate buffering agent is a hydrate.

In some embodiments, borate-polyol complexes include those described in U.S. Pat. No. 6,503,497.

In some embodiments, citrate buffering agents include citric acid and sodium citrate. In some embodiments, the citrate buffering agent comprises citrate.

In some embodiments, acetate buffering agents include acetic acid, potassium acetate, and sodium acetate.

In some embodiments, carbonate buffering agents include sodium bicarbonate and sodium carbonate.

In some embodiments, organic buffering agents include Good's Buffer, such as for example 2-(N-morpholino)ethanesulfonic acid (MES), N-(2-Acetamido)iminodiacetic acid, N-(carbamoylmethyl)iminodiacetic acid (ADA), piperazine-N,N′-bis(2-ethanesulfonic acid (PIPES), N-(2-acetamido)-2-aminoethanesulfonic acid (ACES), β-Hydroxy-4-morpholinepropanesulfonic acid, 3-Morpholino-2-hydroxypropanesulfonic acid (MOPSO), cholamine chloride, 3-(N-morpholino)propansulfonic acid (MOPS), N,N-bis(2-hydroxyethyl)-2-aminoethanesulfonic acid (BES), 2-[(2-Hydroxy-1,1-bis(hydroxymethyl)ethyl)amino]ethanesulfonic acid (TES), 4-(2-hydroxyethyl)-1-piperazineethanesulfonic acid (HEPES), 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), piperazine-1,4,-bis (2-hydroxypropanesulphonic acid) (POPSO), 4-(2-hydroxyethyl)piperazine-1-(2-hydroxypropanesulfonic acid) hydrate (HEPPSO), 3-[4-(2-hydroxyethyl)-1-piperazinyl]propanesulfonic acid (HEPPS), tricine, glycinamide, bicine or N-tris(hydroxymethyl)methyl-3-aminopropanesulfonic acid sodium (TAPS); glycine; and diethanolamine (DEA).

In some embodiments, amino acid buffering agents include taurine, aspartic acid and its salts (e.g., potassium salts, etc.), E-aminocaproic acid, and the like.

Provided herein, in some embodiments, is a pharmaceutical composition essentially free of a citrate buffering agent, an acetate buffering agent, or a combination thereof. In some embodiments, the pharmaceutical composition is substantially free of a citrate buffering agent, an acetate buffering agent, or a combination thereof. In some embodiments, the pharmaceutical composition has no detectable amount of a citrate buffering agent, an acetate buffering agent, or a combination thereof.

In some embodiments, the pharmaceutical composition described herein further comprises a pH adjusting agent. In some embodiments, the pH adjusting agent used is an acid or a base. In some embodiments, the base is selected from oxides, hydroxides, carbonates, bicarbonates, and the likes. In some embodiments, the oxides are metal oxides such as calcium oxide, magnesium oxide, and the likes; hydroxides are of alkali metals and alkaline earth metals such as sodium hydroxide, potassium hydroxide, calcium hydroxide, and the like; and carbonates are sodium carbonate, sodium bicarbonates, potassium bicarbonates, and the like. In some embodiments, the acid is a mineral acid or an organic acid such as hydrochloric acid, nitric acid, phosphoric acid, acetic acid, citric acid, fumaric acid, malic acid, tartaric acid, and the like. In some embodiments, the pH adjusting agent includes, but is not limited to, acetate, bicarbonate, ammonium chloride, citrate, phosphate, pharmaceutically acceptable salts thereof, and combinations or mixtures thereof. In some embodiments, the pH adjusting agent comprises HCl, NaOH, or combinations thereof.

In some embodiments, the pharmaceutical composition has a pH of between about 6 and about 9, about 6.5 to about 8.9, about 7.0 and about 8.8, about 7 and about 8.5, or about 7.5 and about 8. In some embodiments, the pharmaceutical composition has a pH of about 8.0. In some embodiments, the pharmaceutical composition has a pH of about 8.1. In some embodiments, the pharmaceutical composition has a pH of about 8.2. In some embodiments, the pharmaceutical composition has a pH of about 8.3. In some embodiments, the pharmaceutical composition has a pH of about 8.4. In some embodiments, the pharmaceutical composition has a pH of greater than about 6.5. In some embodiments, the pharmaceutical composition has a pH of greater than about 6.6. In some embodiments, the pharmaceutical composition has a pH of greater than about 6.7. In some embodiments, the pharmaceutical composition has a pH of greater than about 6.8. In some embodiments, the pharmaceutical composition has a pH of greater than about 6.9. In some embodiments, the pharmaceutical composition has a pH of greater than about 7.0. In some embodiments, the pharmaceutical composition has a pH of greater than about 7.1. In some embodiments, the pharmaceutical composition has a pH of greater than about 7.2. In some embodiments, the pharmaceutical composition has a pH of greater than about 7.3. In some embodiments, the pharmaceutical composition has a pH of greater than about 7.4. In some embodiments, the pharmaceutical composition has a pH of greater than about 7.5. In some embodiments, the pharmaceutical composition has a pH of greater than about 7.6. In some embodiments, the pharmaceutical composition has a pH of greater than about 7.7. In some embodiments, the pharmaceutical composition has a pH of greater than about 7.8. In some embodiments, the pharmaceutical composition has a pH of greater than about 7.9. In some embodiments, the pharmaceutical composition has a pH of greater than about 8.0. In some embodiments, the pharmaceutical composition has a pH of greater than about 8.1. In some embodiments, the pharmaceutical composition has a pH of greater than about 8.2. In some embodiments, the pharmaceutical composition has a pH of greater than about 8.3. In some embodiments, the pharmaceutical composition has a pH of greater than about 8.4. In some embodiments, the pharmaceutical composition has a pH of greater than about 8.5. In some embodiments, the pharmaceutical composition has a pH of greater than about 8.6. In some embodiments, the pharmaceutical composition has a pH of greater than about 8.7. In some embodiments, the pharmaceutical composition has a pH of greater than about 8.8. In some embodiments, the pharmaceutical composition has a pH of greater than about 8.9. In some embodiments, the pharmaceutical composition has a pH of greater than about 9.0. In some embodiments, the pH is the pH of the pharmaceutical composition after an extended period of time under a storage condition.

In some embodiments, the pharmaceutical composition has an initial pH of between about 6 and about 9, about 6.5 to about 8.9, about 7.0 and about 8.8, about 7 and about 8.5, or about 7.5 and about 8. In some embodiments, the pharmaceutical composition has an initial pH of about 8.0. In some embodiments, the pharmaceutical composition has an initial pH of about 8.1. In some embodiments, the pharmaceutical composition has an initial pH of about 8.2. In some embodiments, the pharmaceutical composition has an initial pH of about 8.3. In some embodiments, the pharmaceutical composition has an initial pH of about 8.4. In some embodiments, the pharmaceutical composition has an initial pH of about 8.5. In some embodiments, the pharmaceutical composition has an initial pH of greater than about 6.5. In some embodiments, the pharmaceutical composition has an initial pH of greater than about 6.6. In some embodiments, the pharmaceutical composition has an initial pH of greater than about 6.7. In some embodiments, the pharmaceutical composition has an initial pH of greater than about 6.8. In some embodiments, the pharmaceutical composition has an initial pH of greater than about 6.9. In some embodiments, the pharmaceutical composition has an initial pH of greater than about 7.0. In some embodiments, the pharmaceutical composition has an initial pH of greater than about 7.1. In some embodiments, the pharmaceutical composition has an initial pH of greater than about 7.2. In some embodiments, the pharmaceutical composition has an initial pH of greater than about 7.3. In some embodiments, the pharmaceutical composition has an initial pH of greater than about 7.4. In some embodiments, the pharmaceutical composition has an initial pH of greater than about 7.5. In some embodiments, the pharmaceutical composition has an initial pH of greater than about 7.6. In some embodiments, the pharmaceutical composition has an initial pH of greater than about 7.7. In some embodiments, the pharmaceutical composition has an initial pH of greater than about 7.8. In some embodiments, the pharmaceutical composition has an initial pH of greater than about 7.9. In some embodiments, the pharmaceutical composition has an initial pH of greater than about 8.0. In some embodiments, the pharmaceutical composition has an initial pH of greater than about 8.1. In some embodiments, the pharmaceutical composition has an initial pH of greater than about 8.2. In some embodiments, the pharmaceutical composition has an initial pH of greater than about 8.3. In some embodiments, the pharmaceutical composition has an initial pH of greater than about 8.4. In some embodiments, the pharmaceutical composition has an initial pH of greater than about 8.5. In some embodiments, the pharmaceutical composition has an initial pH of greater than about 8.6. In some embodiments, the pharmaceutical composition has an initial pH of greater than about 8.7. In some embodiments, the pharmaceutical composition has an initial pH of greater than about 8.8. In some embodiments, the pharmaceutical composition has an initial pH of greater than about 8.9. In some embodiments, the pharmaceutical composition has an initial pH of greater than about 9.0. In some embodiments, the pH is the pH of the pharmaceutical composition after an extended period of time under a storage condition.

In some embodiments, the pH of the pharmaceutical composition described herein is associated with the stability of the pharmaceutical composition. In some embodiments, a stable pharmaceutical composition has a pH of between about 6 and about 9, about 6.5 to about 8.9, about 7.0 and about 8.8, about 7 and about 8.5, or about 7.5 and about 8. In some embodiments, a stable pharmaceutical composition has a pH of about 8.0. In some embodiments, a stable pharmaceutical composition has a pH of about 8.1. In some embodiments, a stable pharmaceutical composition has a pH of about 8.2. In some embodiments, a stable pharmaceutical composition has a pH of about 8.3. In some embodiments, a stable pharmaceutical composition has a pH of about 8.4. In some embodiments, a stable pharmaceutical composition has a pH of about 8.5. In some embodiments, a stable pharmaceutical composition has a pH of greater than about 6.5. In some embodiments, a stable pharmaceutical composition has a pH of greater than about 6.6. In some embodiments, a stable pharmaceutical composition has a pH of greater than about 6.7. In some embodiments, a stable pharmaceutical composition has a pH of greater than about 6.8. In some embodiments, a stable pharmaceutical composition has a pH of greater than about 6.9. In some embodiments, a stable pharmaceutical composition has a pH of greater than about 7.0. In some embodiments, a stable pharmaceutical composition has a pH of greater than about 7.1. In some embodiments, a stable pharmaceutical composition has a pH of greater than about 7.2. In some embodiments, a stable pharmaceutical composition has a pH of greater than about 7.3. In some embodiments, a stable pharmaceutical composition has a pH of greater than about 7.4. In some embodiments, a stable pharmaceutical composition has a pH of greater than about 7.5. In some embodiments, a stable pharmaceutical composition has a pH of greater than about 7.6. In some embodiments, a stable pharmaceutical composition has a pH of greater than about 7.7. In some embodiments, a stable pharmaceutical composition has a pH of greater than about 7.8. In some embodiments, a stable pharmaceutical composition has a pH of greater than about 7.9. In some embodiments, a stable pharmaceutical composition has a pH of greater than about 8.0. In some embodiments, a stable pharmaceutical composition has a pH of greater than about 8.1. In some embodiments, a stable pharmaceutical composition has a pH of greater than about 8.2. In some embodiments, a stable pharmaceutical composition has a pH of greater than about 8.3. In some embodiments, a stable pharmaceutical composition has a pH of greater than about 8.4. In some embodiments, a stable pharmaceutical composition has a pH of greater than about 8.5. In some embodiments, a stable pharmaceutical composition has a pH of greater than about 8.6. In some embodiments, a stable pharmaceutical composition has a pH of greater than about 8.7. In some embodiments, a stable pharmaceutical composition has a pH of greater than about 8.8. In some embodiments, a stable pharmaceutical composition has a pH of greater than about 8.9. In some embodiments, a stable pharmaceutical composition has a pH of greater than about 9.0.

The pharmaceutical composition described herein, in some embodiments, is substantially free of a preservative. In some embodiments, the pharmaceutical composition is substantially free of a benzalkonium chloride preservative. In some embodiments, the pharmaceutical composition has no detectable amount of a benzalkonium chloride preservative. In some embodiments, the pharmaceutical composition has no detectable amount of a benzalkonium chloride. In some embodiments, the pharmaceutical composition is substantially free of a preservative selected from cetrimonium, sodium perborate, stabilized oxychloro complex, SofZia, polyquaternium-1, chlorobutanol, edetate disodium, polyhexamethylene biguanide, or combinations thereof. In some embodiments, the pharmaceutical composition has no detectable amount of a preservative. In some embodiments, the pharmaceutical composition is substantially free of any preservative.

In some embodiments, the pharmaceutical composition described herein is stored in a plastic container. In some embodiments, the material of the plastic container comprises high density polyethylene (HDPE), low density polyethylene (LDPE), polyethylene terephthalate (PET), polyvinyl chloride (PVC), polypropylene (PP), polystyrene (PS), fluorine treated HDPE, post-consumer resin (PCR), K-resin (SBC), or bioplastic. In some embodiments, the material of the plastic container comprises LDPE.

In some embodiments, the pharmaceutical composition described herein is stored in a plastic container. In some embodiments, the pharmaceutical composition stored in a plastic container has a pH of between about 6 and about 9, about 6.5 to about 8.9, about 7.0 and about 8.8, about 7 and about 8.5, or about 7.5 and about 8. In some embodiments, the pharmaceutical composition stored in a plastic container has a pH of about 8.0. In some embodiments, the pharmaceutical composition stored in a plastic container has a pH of about 8.1. In some embodiments, the pharmaceutical composition stored in a plastic container has a pH of about 8.2. In some embodiments, the pharmaceutical composition stored in a plastic container has a pH of about 8.3. In some embodiments, the pharmaceutical composition stored in a plastic container has a pH of about 8.4. In some embodiments, the pharmaceutical composition stored in a plastic container has a pH of about 8.5. In some embodiments, the pharmaceutical composition stored in a plastic container has a pH of greater than about 6.5. In some embodiments, the pharmaceutical composition stored in a plastic container has a pH of greater than about 6.6. In some embodiments, the pharmaceutical composition stored in a plastic container has a pH of greater than about 6.7. In some embodiments, the pharmaceutical composition stored in a plastic container has a pH of greater than about 6.8. In some embodiments, the pharmaceutical composition stored in a plastic container has a pH of greater than about 6.9. In some embodiments, the pharmaceutical composition stored in a plastic container has a pH of greater than about 7.0. In some embodiments, the pharmaceutical composition stored in a plastic container has a pH of greater than about 7.1. In some embodiments, the pharmaceutical composition stored in a plastic container has a pH of greater than about 7.2. In some embodiments, the pharmaceutical composition stored in a plastic container has a pH of greater than about 7.3. In some embodiments, the pharmaceutical composition stored in a plastic container has a pH of greater than about 7.4. In some embodiments, the pharmaceutical composition stored in a plastic container has a pH of greater than about 7.5. In some embodiments, the pharmaceutical composition stored in a plastic container has a pH of greater than about 7.6. In some embodiments, the pharmaceutical composition stored in a plastic container has a pH of greater than about 7.7. In some embodiments, the pharmaceutical composition stored in a plastic container has a pH of greater than about 7.8. In some embodiments, the pharmaceutical composition stored in a plastic container has a pH of greater than about 7.9. In some embodiments, the pharmaceutical composition stored in a plastic container has a pH of greater than about 8.0. In some embodiments, the pharmaceutical composition stored in a plastic container has a pH of greater than about 8.1. In some embodiments, the pharmaceutical composition stored in a plastic container has a pH of greater than about 8.2. In some embodiments, the pharmaceutical composition stored in a plastic container has a pH of greater than about 8.3. In some embodiments, the pharmaceutical composition stored in a plastic container has a pH of greater than about 8.4. In some embodiments, the pharmaceutical composition stored in a plastic container has a pH of greater than about 8.5. In some embodiments, the pharmaceutical composition stored in a plastic container has a pH of greater than about 8.6. In some embodiments, the pharmaceutical composition stored in a plastic container has a pH of greater than about 8.7. In some embodiments, the pharmaceutical composition stored in a plastic container has a pH of greater than about 8.8. In some embodiments, the pharmaceutical composition stored in a plastic container has a pH of greater than about 8.9. In some embodiments, the pharmaceutical composition has a pH of greater than about 9.0.

In some embodiments, the pharmaceutical composition stored in a plastic container has a potency of at least 80% after an extended period of time under a storage condition. In some embodiments, the pharmaceutical composition stored in a plastic container has a potency of at least 85% after an extended period of time under a storage condition. In some embodiments, the pharmaceutical composition stored in a plastic container has a potency of at least 90% after an extended period of time under a storage condition. In some embodiments, the pharmaceutical composition stored in a plastic container has a potency of at least 93% after an extended period of time under a storage condition. In some embodiments, the pharmaceutical composition stored in a plastic container has a potency of at least 95% after an extended period of time under a storage condition. In some embodiments, the pharmaceutical composition stored in a plastic container has a potency of at least 97% after an extended period of time under a storage condition. In some embodiments, the pharmaceutical composition stored in a plastic container has a potency of at least 98% after an extended period of time under a storage condition. In some embodiments, the pharmaceutical composition stored in a plastic container has a potency of at least 99% after an extended period of time under a storage condition. In some embodiments, the storage condition comprises a temperature of about 25° C., about 40° C., or about 60° C. In some embodiments, the extended period of time is at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months.

In some embodiments, the pharmaceutical composition stored in a plastic container has a potency of at least 80% at a temperature of about 0° C., about 2° C., about 5° C., about 10° C., about 15° C., about 25° C., about 40° C., or about 60° C. In some embodiments, the pharmaceutical composition stored in a plastic container has a potency of at least 85% at a temperature of about 0° C., about 2° C., about 5° C., about 10° C., about 15° C., about 25° C., about 40° C., or about 60° C. In some embodiments, the pharmaceutical composition stored in a plastic container has a potency of at least 90% at a temperature of about 0° C., about 2° C., about 5° C., about 10° C., about 15° C., about 25° C., about 40° C., or about 60° C. In some embodiments, the pharmaceutical composition stored in a plastic container has a potency of at least 93% at a temperature of about 0° C., about 2° C., about 5° C., about 10° C., about 15° C., about 25° C., about 40° C., or about 60° C. In some embodiments, the pharmaceutical composition stored in a plastic container has a potency of at least 95% at a temperature of about 0° C., about 2° C., about 5° C., about 10° C., about 15° C., about 25° C., about 40° C., or about 60° C. In some embodiments, the pharmaceutical composition stored in a plastic container has a potency of at least 97% at a temperature of about 0° C., about 2° C., about 5° C., about 10° C., about 15° C., about 25° C., about 40° C., or about 60° C. In some embodiments, the pharmaceutical composition stored in a plastic container has a potency of at least 98% at a temperature of about 0° C., about 2° C., about 5° C., about 10° C., about 15° C., about 25° C., about 40° C., or about 60° C. In some embodiments, the pharmaceutical composition stored in a plastic container has a potency of at least 99% at a temperature of about 0° C., about 2° C., about 5° C., about 10° C., about 15° C., about 25° C., about 40° C., or about 60° C. In some embodiments, the pharmaceutical composition stored in a plastic container has a potency of at least 80%, at least 85%, at least 90%, at least 93%, at least 95%, at least 97%, at least 98%, or at least 99% at a temperature of from about 0° C. to about 30° C., 2° C. to about 10° C. or from about 16° C. to about 26° C.

In some embodiments, the pharmaceutical composition stored in a plastic container has a potency of at least 80% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 85% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 90% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 93% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 95% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 97% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 98% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 99% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months.

In some embodiments, the pharmaceutical composition described herein is formulated as an aqueous solution. In some embodiments, the aqueous solution is a stable aqueous solution. In some embodiments, the aqueous solution is stored in a plastic container as described above. In some embodiments, the aqueous solution is not stored in a glass container. In some embodiments, the aqueous solution is stored in the dark. In some embodiments, the aqueous solution is stored in the presence of light. In some embodiments, the aqueous solution is stable in the presence of light.

In some embodiments, the ophthalmically acceptable pharmaceutical formulations described herein are stable with respect to compound degradation (e.g. less than 30% degradation, less than 25% degradation, less than 20% degradation, less than 15% degradation, less than 10% degradation, less than 8% degradation, less than 5% degradation, less than 3% degradation, less than 2% degradation, or less than 5% degradation) over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months under storage conditions (e.g. room temperature). In other embodiments, the formulations described herein are stable with respect to compound degradation over a period of at least about 1 week. Also described herein are formulations that are stable with respect to compound degradation over a period of at least about 1 month.

### Aqueous Solution Dose-to-Dose Uniformity

Typically, ophthalmic aqueous solutions are packaged in eye drop bottles and administered as drops. For example, a single administration (i.e. a single dose) of an ophthalmic aqueous solution includes a single drop, two drops, three drops, or more into the eyes of the patient. In some embodiments, one dose of the ophthalmic aqueous solution described herein is one drop of the aqueous solution composition from the eye drop bottle.

In some embodiments, described herein are ophthalmic pharmaceutical compositions which provide a dose-to-dose uniform concentration. In some embodiments, the dose-to-dose uniform concentration does not present significant variations of drug content from one dose to another. In some embodiments, the dose-to-dose uniform concentration does provide consistent drug content from one dose to another.

In some embodiments, the pharmaceutical composition has a dose-to-dose ophthalmic agent concentration variation of less than 50%. In some embodiments, the pharmaceutical composition has a dose-to-dose ophthalmic agent concentration variation of less than 40%. In some embodiments, the pharmaceutical composition has a dose-to-dose ophthalmic agent concentration variation of less than 30%. In some embodiments, the pharmaceutical composition has a dose-to-dose ophthalmic agent concentration variation of less than 20%. In some embodiments, the pharmaceutical composition has a dose-to-dose ophthalmic agent concentration variation of less than 10%. In some embodiments, the pharmaceutical composition has a dose-to-dose ophthalmic agent concentration variation of less than 5%.

In some embodiments, the dose-to-dose ophthalmic agent concentration variation is based on 10 consecutive doses. In some embodiments, the dose-to-dose ophthalmic agent concentration variation is based on 8 consecutive doses. In some embodiments, the dose-to-dose ophthalmic agent concentration variation is based on 5 consecutive doses. In some embodiments, the dose-to-dose ophthalmic agent concentration variation is based on 3 consecutive doses. In some embodiments, the dose-to-dose ophthalmic agent concentration variation is based on 2 consecutive doses.

### Sterility

In some embodiments, the pharmaceutical compositions are sterilized. Included within the embodiments disclosed herein are means and processes for sterilization of a pharmaceutical composition disclosed herein for use in humans. The U. S. Food and Drug Administration has provided regulatory guidance in the publication “Guidance for Industry: Sterile Drug Products Produced by Aseptic Processing” available at: http://www.fda.gov/cder/guidance/5882fnl.htm, which is incorporated herein by reference in its entirety.

As used herein, sterilization means a process used to destroy or remove microorganisms that are present in a product or packaging. Any suitable method available for sterilization of objects and compositions is used. Available methods for the inactivation of microorganisms include, but are not limited to, the application of extreme heat, lethal chemicals, or gamma radiation. In some embodiments, a process for the preparation of an ophthalmic formulation comprises subjecting the formulation to a sterilization method selected from heat sterilization, chemical sterilization, radiation sterilization, or filtration sterilization. The method used depends largely upon the nature of the device or composition to be sterilized. Detailed descriptions of many methods of sterilization are given in Chapter 40 of Remington: The Science and Practice of Pharmacy published by Lippincott, Williams & Wilkins, and is incorporated by reference with respect to this subject matter. Filtration

Filtration sterilization is a method used to remove but not destroy microorganisms from solutions. Membrane filters are used to filter heat-sensitive solutions. Such filters are thin, strong, homogenous polymers of mixed cellulosic esters (MCE), polyvinylidene fluoride (PVF; also known as PVDF), or polytetrafluoroethylene (PTFE) and have pore sizes ranging from 0.1 to 0.22 μm. Solutions of various characteristics are optionally filtered using different filter membranes. For example, PVF and PTFE membranes are well suited to filtering organic solvents while aqueous solutions are filtered through PVF or MCE membranes. Filter apparatus are available for use on many scales ranging from the single point-of-use disposable filter attached to a syringe up to commercial scale filters for use in manufacturing plants. The membrane filters are sterilized by autoclave or chemical sterilization. Validation of membrane filtration systems is performed following standardized protocols (Microbiological Evaluation of Filters for Sterilizing Liquids, Vol 4, No. 3. Washington, D.C.: Health Industry Manufacturers Association, 1981) and involve challenging the membrane filter with a known quantity (ca. 10⁷/cm²) of unusually small microorganisms, such as Brevundimonas diminuta (ATCC 19146).

Pharmaceutical compositions are optionally sterilized by passing through membrane filters. In some embodiments, the methods disclosed herein comprise sterilizing the formulation (or components thereof) by means of filtration sterilization.

### Radiation Sterilization

One advantage of radiation sterilization is the ability to sterilize many types of products without heat degradation or other damage. The radiation commonly employed is beta radiation or alternatively, gamma radiation from a ⁶⁰Co source. The penetrating ability of gamma radiation allows its use in the sterilization of many product types, including solutions, compositions, and heterogeneous mixtures. The germicidal effects of irradiation arise from the interaction of gamma radiation with biological macromolecules. This interaction generates charged species and free-radicals. Subsequent chemical reactions, such as rearrangements and cross-linking processes, result in the loss of normal function for these biological macromolecules. The formulations described herein are also optionally sterilized using beta irradiation.

### Sterilization by Heat

Many methods are available for sterilization by the application of high heat. One method is through the use of a saturated steam autoclave. In this method, saturated steam at a temperature of at least 121° C. is allowed to contact the object to be sterilized. The transfer of heat is either directly to the microorganism, in the case of an object to be sterilized, or indirectly to the microorganism by heating the bulk of an aqueous solution to be sterilized. This method is widely practiced as it allows flexibility, safety, and economy in the sterilization process.

### Microorganisms

In some embodiments, the pharmaceutical compositions are substantially free of microorganisms. Acceptable bioburden or sterility levels are based on applicable standards that define therapeutically acceptable compositions. For example, acceptable sterility (e.g., bioburden) levels include about 10 colony forming units (cfu) per gram of formulation, about 50 cfu per gram of formulation, about 100 cfu per gram of formulation, about 500 cfu per gram of formulation or about 1000 cfu per gram of formulation. In some embodiments, acceptable bioburden levels or sterility for formulations include less than 10 cfu/mL, less than 50 cfu/mL, less than 500 cfu/mL or less than 1000 cfu/mL microbial agents. In addition, acceptable bioburden levels or sterility include the exclusion of specified objectionable microbiological agents. By way of example, specified objectionable microbiological agents include but are not limited to Escherichia coli (E. coli), Salmonella sp., Pseudomonas aeruginosa (P. aeruginosa) and/or other specific microbial agents.

An important component of the sterility assurance quality control, quality assurance, and validation process is the method of sterility testing. Sterility testing, by way of example only, is performed by two methods. The first is direct inoculation wherein a sample of the pharmaceutical composition to be tested is added to growth medium and incubated for a period of time up to 21 days. Turbidity of the growth medium indicates contamination. Drawbacks to this method include the small sampling size of bulk materials which reduces sensitivity, and detection of microorganism growth based on a visual observation. An alternative method is membrane filtration sterility testing. In this method, a volume of product is passed through a small membrane filter paper. The filter paper is then placed into media to promote the growth of microorganisms. This method has the advantage of greater sensitivity as the entire bulk product is sampled. The commercially available Millipore Steritest sterility testing system is optionally used for determinations by membrane filtration sterility testing.

Testing for E. coli and Salmonella includes the use of lactose broths incubated at 30-35° C. for 24-72 hours, incubation in MacConkey and/or EMB agars for 18-24 hours, and/or the use of Rappaport medium. Testing for the detection of P. aeruginosa includes the use of NAC agar.

In certain embodiments, the ophthalmic pharmaceutical composition described herein has less than about 60 colony forming units (CFU), less than about 50 colony forming units, less than about 40 colony forming units, or less than about 30 colony forming units of microbial agents per gram of formulation. In certain embodiments, the ophthalmic pharmaceutical composition described herein is formulated to be isotonic with the eye.

### Endotoxins

An additional aspect of the sterilization process is the removal of by-products from the killing of microorganisms (hereinafter, “Product”). The process of depyrogenation removes pyrogens from the sample. Pyrogens are endotoxins or exotoxins which induce an immune response. An example of an endotoxin is the lipopolysaccharide (LPS) molecule found in the cell wall of gram-negative bacteria. While sterilization procedures such as autoclaving or treatment with ethylene oxide kill the bacteria, the LPS residue induces a proinflammatory immune response, such as septic shock. Because the molecular size of endotoxins varies widely, the presence of endotoxins is expressed in “endotoxin units” (EU). One EU is equivalent to 100 picograms of E. coli LPS. In some embodiments, humans develop a response to as little as 5 EU/kg of body weight. The bioburden (e.g., microbial limit) and/or sterility (e.g., endotoxin level) is expressed in any units as recognized in the art. In certain embodiments, ophthalmic pharmaceutical compositions described herein contain lower endotoxin levels (e.g. <4 EU/kg of body weight of a subject) when compared to conventionally acceptable endotoxin levels (e.g., 5 EU/kg of body weight of a subject). In some embodiments, the ophthalmic pharmaceutical composition has less than about 5 EU/kg of body weight of a subject. In other embodiments, the ophthalmic pharmaceutical composition has less than about 4 EU/kg of body weight of a subject. In additional embodiments, the ophthalmic pharmaceutical composition has less than about 3 EU/kg of body weight of a subject. In additional embodiments, the ophthalmic pharmaceutical composition has less than about 2 EU/kg of body weight of a subject.

In some embodiments, the ophthalmic pharmaceutical composition has less than about 5 EU/kg of pharmaceutical composition. In other embodiments, the ophthalmic pharmaceutical composition has less than about 4 EU/kg of pharmaceutical composition. In additional embodiments, the ophthalmic pharmaceutical composition has less than about 3 EU/kg of pharmaceutical composition. In other embodiments, the ophthalmic pharmaceutical composition has less than about 1 EU/kg of pharmaceutical composition. In additional embodiments, the ophthalmic pharmaceutical composition has less than about 0.2 EU/kg of pharmaceutical composition. In certain embodiments, ophthalmic pharmaceutical compositions described herein contain from about 1 to about 5 EU/mL of pharmaceutical composition. In certain embodiments, ophthalmic pharmaceutical compositions described herein contain from about 2 to about 5 EU/mL of pharmaceutical composition, from about 3 to about 5 EU/mL of pharmaceutical composition, or from about 4 to about 5 EU/mL of pharmaceutical composition.

In certain embodiments, ophthalmic pharmaceutical compositions described herein contain lower endotoxin levels (e.g. <0.5 EU/mL of pharmaceutical composition) when compared to conventionally acceptable endotoxin levels (e.g., 0.5 EU/mL of pharmaceutical composition). In some embodiments, the ophthalmic pharmaceutical composition has less than about 0.5 EU/mL of pharmaceutical composition. In other embodiments, the ophthalmic pharmaceutical composition has less than about 0.4 EU/mL of pharmaceutical composition. In additional embodiments, the ophthalmic pharmaceutical composition has less than about 0.2 EU/mL of pharmaceutical composition.

Pyrogen detection, by way of example only, is performed by several methods. Suitable tests for sterility include tests described in United States Pharmacopoeia (USP)<71> Sterility Tests (23rd edition, 1995). The rabbit pyrogen test and the Limulus amebocyte lysate test are both specified in the United States Pharmacopeia Chapters <85> and <151> (USP23/NF 18, Biological Tests, The United States Pharmacopeial Convention, Rockville, Md., 1995). Alternative pyrogen assays have been developed based upon the monocyte activation-cytokine assay. Uniform cell lines suitable for quality control applications have been developed and have demonstrated the ability to detect pyrogenicity in samples that have passed the rabbit pyrogen test and the Limulus amebocyte lysate test (Taktak et al, J. Pharm. Pharmacol. (1990), 43:578-82). In an additional embodiment, the ophthalmic formulation is subject to depyrogenation. In a further embodiment, the process for the manufacture of the ophthalmic pharmaceutical composition comprises testing the pharmaceutical composition for pyrogenicity. In certain embodiments, the pharmaceutical compositions described herein are substantially free of pyrogens.

### Methods of Treatment

Provided herein, in one aspect, is a method for treating an ocular condition of an eye, comprising administering a pharmaceutical composition at, in, or around the eye via a delivery device and per a predetermined dosing regimen, wherein:

the pharmaceutical composition is free of preservatives;

the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%;
   wherein these percentages are with respect to weight per volume; and
the ocular condition is glaucoma, care after cataract surgery, care after LASIK surgery, care for a retina of the eye after cataract surgery, care for a retina of the eye after retina surgery, in preparation for an intraocular procedure, or during the intraocular procedure.

Provided herein, in another aspect, is a method for treating an ocular condition of an eye, comprising administering a pharmaceutical composition at, in, or around the eye via a delivery device and per a predetermined dosing regimen; wherein the pharmaceutical composition comprises at least two active pharmaceutical ingredients compounded and stored in communication with each other; wherein the pharmaceutical composition is free of preservatives; wherein the method is more effective as compared against a preexisting method; wherein the preexisting method administers the at least two active pharmaceutical ingredients from at least two separate and different containers; and wherein the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%;
   wherein these percentages are with respect to weight per volume.

In some embodiments, the ophthalmic pharmaceutical compositions described herein are for use in the treatment of glaucoma, care after cataract surgery, care after LASIK surgery, care for a retina of the eye after cataract surgery, care for a retina of the eye after retina surgery, in preparation for an intraocular procure, or during an intraocular procedure. In some embodiments, the ophthalmic pharmaceutical compositions described herein are for use in the treatment of glaucoma. In some embodiments, the ophthalmic pharmaceutical compositions described herein are for use in care after cataract surgery. In some embodiments, the ophthalmic pharmaceutical compositions described herein are for use in care after LASIK surgery. In some embodiments, the ophthalmic pharmaceutical compositions described herein are for use in care for a retina of the eye after cataract surgery. In some embodiments, the ophthalmic pharmaceutical compositions described herein are for use in care for a retina of the eye after retina surgery. In some embodiments, the ophthalmic pharmaceutical compositions described herein are for use in preparation for an intraocular procedure. In some embodiments, the ophthalmic pharmaceutical compositions described herein are for use during an intraocular procedure.

In some embodiments, the ophthalmic pharmaceutical compositions described herein are packaged in eye drop bottles and administered as drops. For example, a single administration (i.e. a single dose) of an ophthalmic pharmaceutical composition includes a single drop, two drops, three drops or more into the eyes of the patient. In some embodiments, one dose of the ophthalmic pharmaceutical composition described herein is one drop of the aqueous composition from the eye drop bottle.

In some embodiments, the ophthalmic pharmaceutical composition is formulated as an ophthalmic solution for treatment of glaucoma, care after cataract surgery, care after LASIK surgery, care for a retina of the eye after cataract surgery, care for a retina of the eye after retina surgery, in preparation for an intraocular procure, or during an intraocular procedure.

In some embodiments, the ophthalmic pharmaceutical composition is stored below room temperature prior to first use. In some embodiments, the ophthalmic pharmaceutical composition is stored at between about 2° C. to about 10° C. prior to first use. In some embodiments, the ophthalmic pharmaceutical composition is stored at about 2° C., about 3° C., about 4° C., about 5° C., about 6° C., about 7° C., about 8° C., about 9° C., or about 10° C. prior to first use. In some embodiments, the ophthalmic pharmaceutical composition is stored at between about 4° C. to about 8° C. prior to first use.

In some embodiments, the ophthalmic pharmaceutical composition is stored at room temperature after first use. In some embodiments, the ophthalmic pharmaceutical composition is stored at between about 16° C. to about 26° C. after to first use. In some embodiments, the ophthalmic pharmaceutical composition is stored at about 16° C., about 17° C., about 18° C., about 19° C., about 20° C., about 21° C., about 22° C., about 23° C., about 24° C., about 25° C., or about 26° C. after to first use.

In some embodiments, the ophthalmic pharmaceutical compositions are administered as follows: the lower lid of the eye to be administered is pulled down and a predetermined amount of the pharmaceutical composition (e.g. 1-3 drops) is applied to the inside of the eyelid. The ophthalmic tip of the dispensing mechanism does not touch any surface to avoid contamination and/or injury.

In some embodiments, the ophthalmic pharmaceutical composition is administered at predetermined time intervals over an extended period of time. In some embodiments, the ophthalmic pharmaceutical composition is administered once every day. In some embodiments, the ophthalmic pharmaceutical composition is administered every other day. In some embodiments, the ophthalmic pharmaceutical composition is administered over 1 week, 2 weeks, 1 month, 2 months, 3 months, 6 moths, 1 year, 2 years, 3 years, 4 years, 5 years, 6 years, 7 years, 8 years, 9 years, 10 years, 11 years, or 12-15 years.

In some embodiments, the ophthalmic pharmaceutical composition is administered once per day, twice per day, three times per day, once every other day, once per week, once every other week, or once monthly. In some embodiments, the ophthalmic pharmaceutical composition is administered once per day. In some embodiments, the ophthalmic pharmaceutical composition is administered twice per day. In some embodiments, the ophthalmic pharmaceutical composition is administered three times per day. In some embodiments, the ophthalmic pharmaceutical composition is administered once every other day. In some embodiments, the ophthalmic pharmaceutical composition is administered once per week. In some embodiments, the ophthalmic pharmaceutical composition is administered once every other week. In some embodiments, the ophthalmic pharmaceutical composition is administered once monthly.

In some embodiments, the ophthalmic pharmaceutical composition is administered in doses having a dose-to-dose ophthalmic agent concentration variation of less than 50%, less than 40%, less than 30%, less than 20%, less than 10%, or less than 5%.

The number of times a pharmaceutical composition is administered to an individual in need thereof depends on the discretion of a medical professional, the disorder, the severity of the disorder, and the individual's response to the pharmaceutical composition. In some embodiments, a pharmaceutical composition disclosed herein is administered once to an individual in need thereof with a mild acute condition. In some embodiments, a pharmaceutical composition disclosed herein is administered more than once to an individual in need thereof with a moderate or severe acute condition. In the case wherein the patient's condition does not improve, upon the doctor's discretion the administration of an ophthalmic agent is administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the ophthalmic agent is given continuously; alternatively, the dose of drug being administered is temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday is from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's ophthalmic condition has occurred, a maintenance ophthalmic agent dose is administered if necessary. Subsequently, the dosage or the frequency of administration, or both, is optionally reduced, as a function of the symptoms, to a level at which the improved disease, disorder or condition is retained. In certain embodiments, patients require intermittent treatment on a long-term basis upon any recurrence of symptoms.

The amount of ophthalmic agent that will correspond to such an amount will vary depending upon factors such as the particular compound, disease condition and its severity, according to the particular circumstances surrounding the case, including, e.g., the specific ophthalmic agent being administered, the route of administration, the condition being treated, the target area being treated, and the subject or host being treated. The desired dose is presented in a single dose or as divided doses administered simultaneously (or over a short period of time) or at appropriate intervals.

In some embodiments, the initial administration is a particular ophthalmic agent and the subsequent administration a different pharmaceutical composition or ophthalmic agent.

### Delivery Device

In certain embodiments, described herein is an ophthalmic product, which comprises a fluid-dispensing device comprising a reservoir and a dispensing tip fitted onto the reservoir, and the pharmaceutical composition described herein, wherein the pharmaceutical composition is dispensed from the dispensing tip into an eye of an individual in need thereof. In some embodiments, the pharmaceutical composition in the reservoir is substantially preservative-free.

In some embodiments, the ophthalmic product comprises a delivery device. In some embodiments, the delivery device is an eye dropper. In some embodiments, the eye dropper is a multidose eye dropper. In some embodiments, the multidose eye dropper is (i) a dropper bottle for dispensing predetermined metered quantities of liquid, the dropper bottle comprising a non-return position preventing the liquid from flowing back into the dropper bottle; or (ii) an Ophthalmic Squeeze Dispenser (OSD) comprising a sealing closure member that closes a dispenser orifice when the liquid present near the dispenser orifice is at a pressure less than a predetermined threshold. In some embodiments, the multidose eye dropper is a dropper bottle for dispensing predetermined metered quantities of liquid, the dropper bottle comprising a non-return position preventing the liquid from flowing back into the dropper bottle. In some embodiments, the multidose eye dropper is an Ophthalmic Squeeze Dispenser (OSD) comprising a sealing closure member that closes a dispenser orifice when the liquid present near the dispenser orifice is at a pressure less than a predetermined threshold. As used herein, the term “substantially preservative-free” or “substantially free of a preservative” refers to the pharmaceutical composition as having one of: less than about 1%, less than about 0.5%, less than about 0.4%, less than about 0.3%, less than about 0.2%, less than about 0.1%, less than about 0.01%, or less than about 0.001% of a preservative. In some embodiments, the term refers to the pharmaceutical composition as having 0% of a preservative, or preservative-free.

In some embodiments, the ophthalmic product comprises a fluid-dispensing device comprising a reservoir and a dispensing tip fitted onto the reservoir; and an ophthalmic composition comprising about 1% prednisolone PO₄, about 0.5% moxifloxacin HCl, and about 0.075% bromfenac in the reservoir; wherein the ophthalmic composition is dispensed from the dispensing tip into an eye of an individual in need thereof, and wherein the dispensed ophthalmic composition is substantially preservative-free.

In some embodiments, the ophthalmic product comprises a fluid-dispensing device comprising a reservoir and a dispensing tip fitted onto the reservoir; and an ophthalmic composition comprising about 1% prednisolone PO₄ and about 0.5% moxifloxacin HCl in the reservoir; wherein the ophthalmic composition is dispensed from the dispensing tip into an eye of an individual in need thereof, and wherein the dispensed ophthalmic composition is substantially preservative-free.

In some embodiments, the ophthalmic product comprises a fluid-dispensing device comprising a reservoir and a dispensing tip fitted onto the reservoir; and an ophthalmic composition comprising about 0.5% moxifloxacin HCl and about 0.075% bromfenac in the reservoir; wherein the ophthalmic composition is dispensed from the dispensing tip into an eye of an individual in need thereof, and wherein the dispensed ophthalmic composition is substantially preservative-free.

In some embodiments, the ophthalmic product comprises a fluid-dispensing device comprising a reservoir and a dispensing tip fitted onto the reservoir; and an ophthalmic composition comprising about 0.05% difluprednate, about 0.5% moxifloxacin HCl, and about 0.075% bromfenac in the reservoir; wherein the ophthalmic composition is dispensed from the dispensing tip into an eye of an individual in need thereof, and wherein the dispensed ophthalmic composition is substantially preservative-free.

In some embodiments, the reservoir comprises a polymeric material, for example, polyvinyl chloride (PVC) plastics or non-PVC plastics. In some embodiments, the material of the reservoir comprises high-density polyethylene (HDPE), low-density polyethylene (LDPE), polyethylene terephthalate (PET), polyvinyl chloride (PVC), polypropylene (PP), polystyrene (PS), fluorine treated HDPE, post-consumer resin (PCR), K-resin (SBC), or bioplastic. In some embodiments, the material of the reservoir comprises ethylene vinyl acetate (EVA) and block copolymers such as Kraton®. In some embodiments, the material of the reservoir comprises high-density polyethylene (HDPE). In some embodiments, the material of the reservoir comprises low-density polyethylene (LDPE). In some embodiments, the material of the reservoir comprises polyethylene terephthalate (PET). In some embodiments, the material of the reservoir comprises polypropylene (PP). In some embodiments, the material of the reservoir comprises polystyrene (PS). In some embodiments, the material of the reservoir comprises ethylene vinyl acetate (EVA).

In some embodiments, the reservoir further comprises a plasticizer. Exemplary plasticizer includes families of phthalate esters such as di-2-ethylhexylphthalate (DEHP), mono-(2-ethylhexyl) phthalate (MEHP), and triethylhexyltrimellitate (TEHTM); citrate esters such as acetyltri-n-hexyl citrate, acetyltri-n-(hexyl/octyl/decyl) citrate, acetyltri-n-(octyl/decyl) citrate, and n-butyryltri-n-hexyl citrate; and non-phthalate plasticizers such as TEHTM, di(isononyl) cyclohexane-1,2-dicarboxylate (DINCH), or n-butyryltri-n-hexyl citrate.

In some embodiments, the reservoir is at least partially elastically deformable so as to dispense the ophthalmic composition by pressing on the reservoir.

In some embodiments, the reservoir comprises glass.

In some embodiments, the reservoir stores multiple unit doses of the pharmaceutical composition described herein.

In some embodiments, the fluid-dispensing device described herein is a multi-dose fluid-dispensing device.

In some embodiments, the fluid-dispensing device described herein enables storage of a preservative-free or substantially preservative-free composition. In some embodiments, the fluid-dispensing device is a multi-dose preservative-free device.

In some embodiments, a fluid-dispensing device from Aptar Pharma (AptarGroup) is utilized for delivery of a composition described herein. In some embodiments, the pharmaceutical composition is preservative-free.

In some embodiments, a fluid-dispensing device from Nemera La Verpillière S.A.S. is utilized for delivery of a composition described herein. In some embodiments, a fluid-dispensing device as described in U.S. Pat. Nos. 8,986,266 and/or 8,863,998 is utilized for delivery of a composition described herein. In some embodiments, the pharmaceutical composition is preservative-free.

In some embodiments, a fluid-dispensing device from CIS Pharma is utilized for delivery of a composition described herein. In some embodiments, the pharmaceutical composition is preservative-free.

In some embodiments, the fluid-dispensing device described herein optionally comprises an atomizer, a pump, or a mister. In such embodiments, a mechanical system such as a pump, a mister, or an atomizer is incorporated into the fluid-dispensing device to facilitate delivery of the pharmaceutical composition described herein and optionally to facilitate dose uniformity (e.g., between each administration, minimize excessive drug volume, and/or enhance droplet uniformity). In additional embodiments, a mechanical system such as a pump, a mister, or an atomizer is incorporated into the fluid-dispensing device to enhance and/or optimize the amount of drug delivered to the eye.

In some embodiments, an atomizer and/or pump system from Aero Pump GMBH (Adelphi Healthcare Packaging) is utilized with the fluid-dispensing device and the pharmaceutical composition described herein. In some embodiments, a multiple-dosage fluid-dispensing device from Aero Pump GMBH is utilized for delivery of the pharmaceutical composition described herein. In some embodiments, a fluid-dispensing device as described in U.S. Pat. No. 10,155,243 and/or US Patent Publication No. 2015/076174 (Aero Pump GMBH) is utilized with the fluid-dispensing device and the pharmaceutical composition described herein.

In some embodiments, a fluid-dispensing device from Eyenovia, Inc. is utilized for delivery of the pharmaceutical composition described herein. In some embodiments, a fluid-dispensing device comprising one or more of a delivery system and/or component described in U.S. Pat. Nos. 9,539,604, 9,087,145, 9,463,486, or 8,684,980 are utilized for delivery of the pharmaceutical composition described herein.

In some embodiments, a fluid-dispensing device comprising one or more of a delivery system and/or component from Kedalion Therapeutics is utilized for delivery of the pharmaceutical composition described herein.

In some embodiments, a fluid-dispensing device comprising one or more of a delivery system and/or component from Aptar Pharma (e.g., a pump dispensing system) is utilized for delivery of the pharmaceutical composition described herein.

In some embodiments, the fluid-dispensing device optionally comprises an internal filter or membrane. In some embodiments, the internal filter or membrane is located within the fluid-dispensing device at a position capable of removing a microorganism and/or an endotoxin from the ophthalmic composition prior to dispensing the ophthalmic composition into the eye of the individual. In some embodiments, the internal filter or membrane is located at the junction connecting the dispensing tip to the reservoir. In other cases, the internal filter or membrane is located within the dispensing tip. In some embodiments, the ophthalmic composition is a preservative-free composition.

In some embodiments, the internal filter or membrane comprises cellulose acetate, cellulose nitrate, nylon, polyether sulfone (PES), polypropylene (PP), polyvinyl difluoride (PVDF), silicone, polycarbonate, or a combination thereof.

In some embodiments, the dispensed composition comprises one of: less than about 1%, less than about 0.5%, less than about 0.4%, less than about 0.3%, less than about 0.2%, less than about 0.1%, less than about 0.01%, less than about 0.001%, or less than about 0.0001% of a preservative. In some embodiments, the dispensed composition is preservative-free.

In some embodiments, the droplet volume dispensed from the fluid-dispensing device described herein is from about 0.1 μL to about 50 μL. In some embodiments, the droplet volume is one of: about 0.1 μL to about 40 μL, about 0.5 μL to about 30 μL, about 1 μL to about 30 μL, about 5 μL to about 20 μL, about 10 μL to about 20 μL, about 5 μL to about 40 μL, about 5 μL to about 30 μL, about 6 μL to about 8 μL, about 6 μL to about 7 μL, about 7 μL to about 8 μL, about 10 μL to about 40 μL, or about 10 μL to about 30 μL. In some embodiments, the droplet volume dispensed from the fluid-dispensing device described herein is about 0.1 μL, about 0.2 μL, about 0.3 μL, about 0.4 μL, about 0.5 μL, about 1 μL, about 5 μL, about 6 μL, about 7 μL, about 8 μL, about 9 μL, about 10 μL, about 20 μL, about 30 μL, about 40 μL, or about 50 μL.

In some embodiments, the linear size or diameter of the droplet when spherical is about 1 up to less than 100 microns. In some embodiments, the linear size or diameter of the droplet is about 20 to 100 microns, about 1 to 20 microns, 1-15 microns, 1-10 microns, 8-20 microns, 8-15 microns, 8-12 microns, or 1-5 microns. In the context of an aerosol or mist, the size of the droplet is, for example, 1-5 microns, 1-10 microns, less than 10 microns, greater than 10 microns, or up to 100 microns.

In some embodiments, the diameter of the droplet is calculated using the equation V=47cr³ where the diameter=2r.

In some embodiments, the fluid-dispensing device described herein facilitates at least 60%, 70%, 80%, 85%, 90%, 95%, or 99% of the ejected mass of a droplet deposited on the eye of an individual. In some embodiments, the fluid-dispensing device described herein facilitates at least 70% of the ejected mass of a droplet to be deposited on the eye of an individual. In some embodiments, the fluid-dispensing device described herein facilitates at least 80% of the ejected mass of a droplet to be deposited on the eye of an individual. In some embodiments, the fluid-dispensing device described herein facilitates at least 90% of the ejected mass of a droplet to be deposited on the eye of an individual. In some embodiments, the fluid-dispensing device described herein facilitates at least 95% of the ejected mass of a droplet to be deposited on the eye of an individual. In some embodiments, the fluid-dispensing device described herein facilitates at least 99% of the ejected mass of a droplet to be deposited on the eye of an individual.

### Kits/Articles of Manufacture

This disclosure also provides kits for treatment of glaucoma, care after cataract surgery, care after LASIK surgery, care for a retina of the eye after cataract surgery, care for a retina of the eye after retina surgery, in preparation for an intraocular procedure, or during the intraocular procedure. Such kits generally will comprise one or more of the ophthalmic pharmaceutical compositions disclosed herein and instructions for using the kit. This disclosure also contemplates the use of one or more of the ophthalmic pharmaceutical compositions in the manufacture of medicaments for treating, abating, reducing, or ameliorating the symptoms of a disease, dysfunction, or disorder in a mammal, such as a human.

In some embodiments, kits include a carrier, package, or container that is compartmentalized to receive one or more containers such as vials, tubes, or bottles. In other embodiments, the containers are formed from a variety of materials such as glass or plastic.

The articles of manufacture provided herein contain packaging materials. Packaging materials for use in packaging pharmaceutical products are also presented herein. See, e.g., U.S. Pat. Nos. 5,323,907, 5,052,558 and 5,033,252. Examples of pharmaceutical packaging materials include, but are not limited to, dropper bottles, tubes, pumps, bags, vials, containers, syringes, bottles, and any packaging material suitable for a selected formulation and intended mode of administration and treatment. A wide array of ophthalmic pharmaceutical compositions provided herein are contemplated as are a variety of treatments for any disease, disorder, or condition that benefits by controlled release administration of an ophthalmic agent to the eye.

In some embodiments, a kit includes one or more additional containers, each with one or more of various materials (such as rinses, wipes, and/or devices) desirable from a commercial and user standpoint for use of a pharmaceutical composition described herein. Such materials also include labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments, a label is used to indicate that the contents are to be used for a specific therapeutic application. In yet another embodiment, a label also indicates directions for use of the contents, such as in the methods described herein.

In certain embodiments, the ophthalmic pharmaceutical compositions are presented in a dispenser device which contains one or more unit dosage forms containing a pharmaceutical composition provided herein. In a further embodiment, the dispenser device is accompanied by instructions for administration. In yet a further embodiment, the dispenser is also accompanied with a notice associated with the container in form prescribed by a governmental agency regulating the manufacture, use, or sale of pharmaceuticals, which notice is reflective of approval by the agency of the form of the drug for human or veterinary administration. In another embodiment, such notice, for example, is the labeling approved by the U.S. Food and Drug Administration for prescription drugs, or the approved product insert. In yet another embodiment, compositions containing a pharmaceutical composition provided herein formulated in a compatible pharmaceutical carrier are also prepared, placed in an appropriate container, and labeled for treatment of an indicated condition.

### EXAMPLES

### Example 1: Preparation of Prednisolone PO₄ 1%/Moxifloxacin HCl 0.5%/Bromfenac 0.075%

To 1,060 mL sterile water for injection (SWFI) was added moxifloxacin hydrochloride (33.3 g, 76.1 mmol). SWFI was added to bring the solution to a weight of 3,520 g and the resulting suspension was stirred for 10 minutes until all solids were dissolved. The solution was adjusted to pH>7 with 10% sodium hydroxide (30 mL).

In a separate container, to 1,050 mL SWFI were sequentially added boric acid (82.01 g, 1,326 mmol), prednisolone sodium phosphate (75.63 g, 155.5 mmol), and 1,060 mL SWFI. The solution was adjusted to pH>7 with 10% sodium hydroxide (95 mL) and added to the moxifloxacin solution. The resulting solution was adjusted to pH>8 with 10% sodium hydroxide if needed. Bromfenac sodium (5.54 g, 15.6 mmol) was added with stirring, and SWFI was added to bring the solution to a weight of 6,400 g. The solution was adjusted to pH>8 with 10% NaOH, and SWFI was added to bring the solution to a final weight of 6,447.4 g.

### Example 2: Preparation of Prednisolone PO₄ 1%/Moxifloxacin HCl 0.5%

To 3,080 mL sterile water for injection (SWFI) was added moxifloxacin hydrochloride (29.2 g, 66.7 mmol), and the resulting suspension was stirred until all solids were dissolved. The solution was adjusted to pH 7 with 10% sodium hydroxide.

In a separate container, to 1,060 mL SWFI were sequentially added boric acid (70.63 g, 1,142 mmol) and prednisolone sodium phosphate (66.28 g, 136.3 mmol). The solution was adjusted to pH>7 with 10% sodium hydroxide and added to the moxifloxacin solution. SWFI was added to bring the solution to a weight of 5,550 g. The solution was adjusted to pH>7 with 10% NaOH, and SWFI was added to bring the solution to a final weight of 5,650.4 g.

### Example 3: Preparation of Moxifloxacin HCl 0.5%/Bromfenac 0.075%

To 1,060 mL sterile water for injection (SWFI) was added moxifloxacin hydrochloride (33.3 g, 76.1 mmol). SWFI (3,180 mL) was added and the resulting suspension was stirred until all solids were dissolved. Boric acid (108.3 g, 1,752 mmol) was added and SWFI was added to bring the solution to a weight of 6,000 g. The solution was adjusted to pH>8 with 10% sodium hydroxide. Bromfenac sodium (5.54 g, 15.6 mmol) was added with stirring, and the solution was adjusted to pH >8 with 10% NaOH. SWFI was added to bring the solution to a final weight of 6,447.4 g.

### Example 4: Preparation of Difluprednate 0.05%/Moxifloxacin HCl 0.5%/Bromfenac 0.075%

Castor oil (200 g) was added to difluprednate (2.0 g, 3.93 mmol) and the resulting suspension was stirred at 70° C. until transparent and sterilized in an oven.

In a separate container, to 1,060 mL sterile water for injection (SWFI) was added moxifloxacin hydrochloride (20.7 g, 47.3 mmol). SWFI (3,180 mL) was added and the resulting suspension was stirred until all solids were dissolved. Povidone (80.0 g) was added and the resulting suspension was stirred until all solids were dissolved. Edetate disodium (0.80 g, 2.38 mmol) was added and the resulting suspension was stirred until all solids were dissolved. Glycerin (80.0 g, 869 mmol) and polysorbate 80 NF (160.0 g, 122 mmol) were added and the solution was adjusted to pH >8 with 10% sodium hydroxide. Bromfenac (3.464 g, 10.4 mmol) was added and the resulting suspension was stirred until all solids were dissolved. The difluprednate solution was added, the resulting solution was adjusted to pH>8 with 10% sodium hydroxide, and SWFI was added to bring the solution to a final weight of 4,000.0 g. The resulting suspension was emulsified with an autoclave-sterilized homogenizer head at 8,000 RPM for 1 hour.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating an ocular condition of an eye; comprising administering a pharmaceutical composition at, in, or around the eye via a delivery device and per a predetermined dosing regimen, wherein:
the pharmaceutical composition is free of preservatives;
the pharmaceutical composition comprises one of:
(1) timolol maleate 0.5% and latanoprost about 0.005%;
(2) moxifloxacin about 0.5%;
(3) dexamethasone PO₄ about 0.1% and moxifloxacin about 0.5%;
(4) dexamethasone PO₄ about 0.1%, moxifloxacin about 0.5%, and ketorolac tromethamine about 0.5%;
(5) dexamethasone PO₄ about 0.1%, moxifloxacin about 0.5%, and bromfenac about 0.07%;
(6) prednisolone PO₄ about 1% and gatifloxacin about 0.5%;
(7) prednisolone PO₄ about 1% and ketorolac tromethamine about 0.5%;
(8) prednisolone PO₄ about 1%, ketorolac tromethamine about 0.5%, and gatifloxacin about 0.5%;
(9) prednisolone PO₄ about 1% and bromfenac about 0.07%;
(10) prednisolone PO₄ about 1%, gatifloxacin about 0.5%, and bromfenac about 0.07%;
(11) prednisolone PO₄ about 1%, moxifloxacin about 0.5%, and bromfenac about 0.075%;
(12) prednisolone PO₄ about 1% and moxifloxacin about 0.5%;
(13) moxifloxacin about 0.5% and bromfenac about 0.075%;
(14) difluprednate about 0.05%, moxifloxacin about 0.5%, and bromfenac about 0.075%;
wherein these percentages are with respect to weight per volume.

2. The method of claim 1, wherein the ocular condition is care after cataract surgery, care after LASIK surgery, care for a retina of the eye after cataract surgery, care for a retina of the eye after retina surgery, in preparation for an intraocular procedure, or during the intraocular procedure.

3. The method of claim 1, wherein the delivery device is an eye dropper.

4. The method of claim 3, wherein the eye dropper is a multidose eye dropper.

5. The method of claim 4, wherein the multidose eye dropper is (i) a dropper bottle for dispensing predetermined metered quantities of liquid, the dropper bottle comprising a non-return position preventing the liquid from flowing back into the dropper bottle; or (ii) an Ophthalmic Squeeze Dispenser (OSD) comprising a sealing closure member that closes a dispenser orifice when the liquid present near the dispenser orifice is at a pressure less than a predetermined threshold.

6. The method of claim 1, wherein the predetermined dosing regimen is once per day, twice per day, three times per day, once every other day, once per week, once every other week, or once monthly.

7. The method of claim 1, wherein the pharmaceutical composition comprises prednisolone PO₄ about 1%, moxifloxacin about 0.5%, and bromfenac about 0.075%.

8. The method of claim 1, wherein the pharmaceutical composition comprises prednisolone PO₄ about 1% and moxifloxacin about 0.5%.

9. The method of claim 1, wherein the pharmaceutical composition comprises moxifloxacin about 0.5% and bromfenac about 0.075%.

10. The method of claim 1, wherein the pharmaceutical composition comprises difluprednate about 0.05%, moxifloxacin about 0.5%, and bromfenac about 0.075%.

11. A method for treating an ocular condition of an eye; comprising administering a pharmaceutical composition at, in, or around the eye via a delivery device and per a predetermined dosing regimen, wherein:
the pharmaceutical composition is free of preservatives;
the pharmaceutical composition comprises: prednisolone PO₄ about 1%, a fluoroquinolone antibacterial agent about 0.5%, and bromfenac about 0.075%;
wherein these percentages are with respect to weight per volume.

12. The method of claim 11, wherein the fluoroquinolone antibacterial agent is moxifloxacin or gatifloxacin.

13. The method of claim 11, wherein the fluoroquinolone antibacterial agent is moxifloxacin.

14. The method of claim 11, wherein the ocular condition is one or more of: care after cataract surgery, care after LASIK surgery, care for a retina of the eye after cataract surgery, care for a retina of the eye after retina surgery, in preparation for an intraocular procure, or during an intraocular procedure.

15. The method of claim 11, wherein the delivery device is an eye dropper.

16. The method of claim 15, wherein the eye dropper is a multidose eye dropper.

17. The method of claim 16, wherein the multidose eye dropper is (i) a dropper bottle for dispensing predetermined metered quantities of liquid, the dropper bottle comprising a non-return position preventing the liquid from flowing back into the dropper bottle; or (ii) an Ophthalmic Squeeze Dispenser (OSD) comprising a sealing closure member that closes a dispenser orifice when the liquid present near the dispenser orifice is at a pressure less than a predetermined threshold.

18. The method of claim 11, wherein the predetermined dosing regimen is once per day, twice per day, three times per day, once every other day, once per week, once every other week, or once monthly.

19. The method of claim 11, wherein the pharmaceutical composition comprises prednisolone PO₄ about 1%, moxifloxacin about 0.5%, and bromfenac about 0.075%.

20. A delivery device, comprising: a pharmaceutical composition,
wherein the pharmaceutical composition comprises one of:
(1) timolol maleate 0.5% and latanoprost about 0.005%;
(2) moxifloxacin about 0.5%;
(3) dexamethasone PO₄ about 0.1% and moxifloxacin about 0.5%;
(4) dexamethasone PO₄ about 0.1%, moxifloxacin about 0.5%, and ketorolac tromethamine about 0.5%;
(5) dexamethasone PO₄ about 0.1%, moxifloxacin about 0.5%, and bromfenac about 0.07%;
(6) prednisolone PO₄ about 1% and gatifloxacin about 0.5%;
(7) prednisolone PO₄ about 1% and ketorolac tromethamine about 0.5%;
(8) prednisolone PO₄ about 1%, ketorolac tromethamine about 0.5%, and gatifloxacin about 0.5%;
(9) prednisolone PO₄ about 1% and bromfenac about 0.07%;
(10) prednisolone PO₄ about 1%, gatifloxacin about 0.5%, and bromfenac about 0.07%;
(11) prednisolone PO₄ about 1%, moxifloxacin about 0.5%, and bromfenac about 0.075%;
(12) prednisolone PO₄ about 1% and moxifloxacin about 0.5%;
(13) moxifloxacin about 0.5% and bromfenac about 0.075%;
(14) difluprednate about 0.05%, moxifloxacin about 0.5%, and bromfenac about 0.075%;
wherein these percentages are with respect to weight per volume.

21. The delivery device of claim 20, wherein the delivery device is an eye dropper.

22. The delivery device of claim 21, wherein the eye dropper is a multidose eye dropper.

23. The delivery device of claim 22, wherein the multidose eye dropper is (i) a dropper bottle for dispensing predetermined metered quantities of liquid, the dropper bottle comprising a non-return position preventing the liquid from flowing back into the dropper bottle; or (ii) an Ophthalmic Squeeze Dispenser (OSD) comprising a sealing closure member that closes a dispenser orifice when the liquid present near the dispenser orifice is at a pressure less than a predetermined threshold.

24. The delivery device of claim 20, wherein the pharmaceutical composition comprises prednisolone PO₄ about 1%, moxifloxacin about 0.5%, and bromfenac about 0.075%.

25. The delivery device of claim 20, wherein the pharmaceutical composition comprises prednisolone PO₄ about 1% and moxifloxacin about 0.5%.

26. The delivery device of claim 20, wherein the pharmaceutical composition comprises moxifloxacin about 0.5% and bromfenac about 0.075%.

27. The delivery device of claim 20, wherein the pharmaceutical composition comprises difluprednate about 0.05%, moxifloxacin about 0.5%, and bromfenac about 0.075%.
